SEQUENCE LISTING (102 sequences)

SEQ ID NO: 1 - protein, 10 aa, Artificial Sequence
  Synthetic construct
WRWRWRWRWR

SEQ ID NO: 2 - protein, 8 aa, Artificial Sequence
  Synthetic construct
WRWRWRWR

SEQ ID NO: 3 - protein, 4 aa, Artificial Sequence
  Synthetic construct
WRWR

SEQ ID NO: 4 - protein, 12 aa, Artificial Sequence
  Synthetic construct
CRRRRRWWWWWC

SEQ ID NO: 5 - protein, 10 aa, Artificial Sequence
  Synthetic construct
  MOD_RES (5)..(5): bAla
  MOD_RES (10)..(10): bAla
WWWWARRRRA

SEQ ID NO: 6 - protein, 10 aa, Artificial Sequence
  Synthetic construct
  MOD_RES (5)..(5): bAla
  MOD_RES (10)..(10): bAla
WWWWARRRRA

SEQ ID NO: 7 - protein, 12 aa, Artificial Sequence
  Synthetic construct
  MOD_RES (5)..(6): bAla
  MOD_RES (11)..(12): bAla
WWWWAARRRRAA

SEQ ID NO: 8 - protein, 12 aa, Artificial Sequence
  Synthetic construct
  MOD_RES (5)..(6): bAla
  MOD_RES (11)..(12): bAla
WWWWAARRRRAA

SEQ ID NO: 9 - protein, 5 aa, Artificial Sequence
  Synthetic construct
  MISC_FEATURE (4)..(4): d-phenylalanine
RGDXK

SEQ ID NO: 10 - protein, 10 aa, Artificial Sequence
  Synthetic construct
  MOD_RES (5)..(5): bAla
  MOD_RES (10)..(10): bAla
RRRRAWWWWA

SEQ ID NO: 11 - protein, 12 aa, Artificial Sequence
  Synthetic construct
  MOD_RES (5)..(6): bAla
  MOD_RES (11)..(12): bAla
RRRRAAWWWWAA

SEQ ID NO: 12 - protein, 8 aa, Artificial Sequence
  Synthetic construct
RRRRWWWW

SEQ ID NO: 13 - protein, 10 aa, Artificial Sequence
  Synthetic construct
RRRRRWWWWW

SEQ ID NO: 14 - protein, 15 aa, Artificial Sequence
  Synthetic construct
WWWWCRRRRRCWWWW

SEQ ID NO: 15 - protein, 8 aa, Artificial Sequence
  Synthetic construct
CRWRWRWC

SEQ ID NO: 16 - protein, 10 aa, Artificial Sequence
  Synthetic construct
CRRRRWWWWC

SEQ ID NO: 17 - protein, 11 aa, Artificial Sequence
  Synthetic construct
WWCRRRRRCWW

SEQ ID NO: 18 - protein, 11 aa, Artificial Sequence
  Synthetic construct
RRCWWWWWCRR

SEQ ID NO: 19 - protein, 12 aa, Artificial Sequence
  Synthetic construct
  MOD_RES (6)..(6): bAla
  MOD_RES (12)..(12): bAla
WWWWWARRRRRA

SEQ ID NO: 20 - protein, 10 aa, Artificial Sequence
  Synthetic construct
CRWRWRWRWC

SEQ ID NO: 21 - protein, 11 aa, Artificial Sequence
  Synthetic construct
CRRRRRWWWWC

SEQ ID NO: 22 - protein, 9 aa, Artificial Sequence
  Synthetic construct
RRRRRWWWW

SEQ ID NO: 23 - protein, 12 aa, Artificial Sequence
  Synthetic construct
WWCRRRRRRCWW

SEQ ID NO: 24 - protein, 12 aa, Artificial Sequence
  Synthetic construct
RRCWWWWWWCRR

SEQ ID NO: 25 - protein, 12 aa, Artificial Sequence
  Synthetic construct
CRWRWRWRWRWC

SEQ ID NO: 26 - protein, 12 aa, Artificial Sequence
  Synthetic construct
CRRRRRRWWWWC

SEQ ID NO: 27 - protein, 10 aa, Artificial Sequence
  Synthetic construct
RRRRRRWWWW

SEQ ID NO: 28 - protein, 12 aa, Artificial Sequence
  Synthetic construct
WWCRRRRRRRWW

SEQ ID NO: 29 - protein, 13 aa, Artificial Sequence
  Synthetic construct
RRCWWWWWWWCRR

SEQ ID NO: 30 - protein, 8 aa, Artificial Sequence
  Synthetic construct
  MOD_RES (4)..(4): bAla
  MOD_RES (8)..(8): bAla
WWWARRRA

SEQ ID NO: 31 - protein, 14 aa, Artificial Sequence
  Synthetic construct
CRWRWRWRWRWRWC

SEQ ID NO: 32 - protein, 13 aa, Artificial Sequence
  Synthetic construct
CRRRRRRRWWWWC

SEQ ID NO: 33 - protein, 11 aa, Artificial Sequence
  Synthetic construct
RRRRRRRWWWW

SEQ ID NO: 34 - protein, 13 aa, Artificial Sequence
  Synthetic construct
WWWCRRRRRCWWW

SEQ ID NO: 35 - protein, 13 aa, Artificial Sequence
  Synthetic construct
RRRCWWWWWCRRR

SEQ ID NO: 36 - protein, 6 aa, Artificial Sequence
  Synthetic construct
  MOD_RES (3)..(3): bAla
  MOD_RES (6)..(6): bAla
WWARRA

SEQ ID NO: 37 - protein, 16 aa, Artificial Sequence
  Synthetic construct
CRWRWRWRWRWRWRWC

SEQ ID NO: 38 - protein, 11 aa, Artificial Sequence
  Synthetic construct
CRRRRWWWWWC

SEQ ID NO: 39 - protein, 9 aa, Artificial Sequence
  Synthetic construct
RRRRWWWWW

SEQ ID NO: 40 - protein, 14 aa, Artificial Sequence
  Synthetic construct
WWWCRRRRRRCWWW

SEQ ID NO: 41 - protein, 15 aa, Artificial Sequence
  Synthetic construct
RRRCWWWWWWWCRRR

SEQ ID NO: 42 - protein, 12 aa, Artificial Sequence
  Synthetic construct
  MOD_RES (5)..(6): bAla
  MOD_RES (11)..(12): bAla
WWWWAARRRRAA

SEQ ID NO: 43 - protein, 11 aa, Artificial Sequence
  Synthetic construct
CRWWWWWWWWC

SEQ ID NO: 44 - protein, 12 aa, Artificial Sequence
  Synthetic construct
CRRRRWWWWWWC

SEQ ID NO: 45 - protein, 10 aa, Artificial Sequence
  Synthetic construct
RRRRWWWWWW

SEQ ID NO: 46 - protein, 15 aa, Artificial Sequence
  Synthetic construct
WWWCRRRRRRRCWWW

SEQ ID NO: 47 - protein, 15 aa, Artificial Sequence
  Synthetic construct
RRRCWWWWWWWCRRR

SEQ ID NO: 48 - protein, 12 aa, Artificial Sequence
  Synthetic construct
  MOD_RES (6)..(6): bAla
  MOD_RES (12)..(12): bAla
WWWWWARRRRRA

SEQ ID NO: 49 - protein, 20 aa, Artificial Sequence
  Synthetic construct
CRWRWRWRWRWRWRWRWRWC

SEQ ID NO: 50 - protein, 13 aa, Artificial Sequence
  Synthetic construct
CRRRRWWWWWWWC

SEQ ID NO: 51 - protein, 11 aa, Artificial Sequence
  Synthetic construct
RRRRWWWWWWW

SEQ ID NO: 52 - protein, 15 aa, Artificial Sequence
  Synthetic construct
WWWWCRRRRRCWWWW

SEQ ID NO: 53 - protein, 15 aa, Artificial Sequence
  Synthetic construct
RRRRCWWWWWCRRRR

SEQ ID NO: 54 - protein, 10 aa, Artificial Sequence
  Synthetic construct
  MOD_RES (5)..(5): bAla
  MOD_RES (10)..(10): bAla
WWWWARRRRA

SEQ ID NO: 55 - protein, 22 aa, Artificial Sequence
  Synthetic construct
CRWRWRWRWRWRWRWRWRWRWC

SEQ ID NO: 56 - protein, 12 aa, Artificial Sequence
  Synthetic construct
CRRRRRWWWWWC

SEQ ID NO: 57 - protein, 10 aa, Artificial Sequence
  Synthetic construct
RRRRRWWWWW

SEQ ID NO: 58 - protein, 16 aa, Artificial Sequence
  Synthetic construct
WWWWCRRRRRRCWWWW

SEQ ID NO: 59 - protein, 16 aa, Artificial Sequence
  Synthetic construct
RRRRCWWWWWWCRRRR

SEQ ID NO: 60 - protein, 8 aa, Artificial Sequence
  Synthetic construct
  MOD_RES (4)..(4): bAla
  MOD_RES (8)..(8): bAla
WWWARRRA

SEQ ID NO: 61 - protein, 10 aa, Artificial Sequence
  Synthetic construct
RWRWRWRWRW

SEQ ID NO: 62 - protein, 13 aa, Artificial Sequence
  Synthetic construct
CRRRRRRWWWWWC

SEQ ID NO: 63 - protein, 11 aa, Artificial Sequence
  Synthetic construct
RRRRRRWWWWW

SEQ ID NO: 64 - protein, 17 aa, Artificial Sequence
  Synthetic construct
WWWWCRRRRRRRCWWWW

SEQ ID NO: 65 - protein, 17 aa, Artificial Sequence
  Synthetic construct
RRRRCWWWWWWWCRRRR

SEQ ID NO: 66 - protein, 6 aa, Artificial Sequence
  Synthetic construct
  MOD_RES (3)..(3): bAla
  MOD_RES (6)..(6): bAla
WWARRA

SEQ ID NO: 67 - protein, 12 aa, Artificial Sequence
  Synthetic construct
KRWRWRWRWRWE

SEQ ID NO: 68 - protein, 14 aa, Artificial Sequence
  Synthetic construct
CRRRRRRRWWWWWC

SEQ ID NO: 69 - protein, 12 aa, Artificial Sequence
  Synthetic construct
RRRRRRRWWWWW

SEQ ID NO: 70 - protein, 16 aa, Artificial Sequence
  Synthetic construct
WWWWWCRRRRRWWWWW

SEQ ID NO: 71 - protein, 17 aa, Artificial Sequence
  Synthetic construct
RRRRRCWWWWWCRRRRR

SEQ ID NO: 72 - protein, 12 aa, Artificial Sequence
  Synthetic construct
  MOD_RES (5)..(6): bAla
  MOD_RES (11)..(12): bAla
WWWWAARRRRAA

SEQ ID NO: 73 - protein, 13 aa, Artificial Sequence
  Synthetic construct
CRRRRRWWWWWWC

SEQ ID NO: 74 - protein, 11 aa, Artificial Sequence
  Synthetic construct
RRRRRWWWWWW

SEQ ID NO: 75 - protein, 18 aa, Artificial Sequence
  Synthetic construct
WWWWWCRRRRRRCWWWWW

SEQ ID NO: 76 - protein, 18 aa, Artificial Sequence
  Synthetic construct
RRRRRCWWWWWWCRRRRR

SEQ ID NO: 77 - protein, 14 aa, Artificial Sequence
  Synthetic construct
CRRRRRWWWWWWWC

SEQ ID NO: 78 - protein, 12 aa, Artificial Sequence
  Synthetic construct
RRRRRWWWWWWW

SEQ ID NO: 79 - protein, 19 aa, Artificial Sequence
  Synthetic construct
WWWWWCRRRRRRRCWWWWW

SEQ ID NO: 80 - protein, 19 aa, Artificial Sequence
  Synthetic construct
RRRRRCWWWWWWWCRRRRR

SEQ ID NO: 81 - protein, 14 aa, Artificial Sequence
  Synthetic construct
CRRRRRRWWWWWWC

SEQ ID NO: 82 - protein, 12 aa, Artificial Sequence
  Synthetic construct
RRRRRRWWWWWW

SEQ ID NO: 83 - protein, 16 aa, Artificial Sequence
  Synthetic construct
CRRRRRRRWWWWWWWC

SEQ ID NO: 84 - protein, 14 aa, Artificial Sequence
  Synthetic construct
RRRRRRRWWWWWWW

SEQ ID NO: 85 - protein, 18 aa, Artificial Sequence
  Synthetic construct
CRRRRRRRRWWWWWWWWC

SEQ ID NO: 86 - protein, 16 aa, Artificial Sequence
  Synthetic construct
RRRRRRRRWWWWWWWW

SEQ ID NO: 87 - protein, 20 aa, Artificial Sequence
  Synthetic construct
CRRRRRRRRRWWWWWWWWWC

SEQ ID NO: 88 - protein, 18 aa, Artificial Sequence
  Synthetic construct
RRRRRRRRRWWWWWWWWW

SEQ ID NO: 89 - protein, 22 aa, Artificial Sequence
  Synthetic construct
CRRRRRRRRRRWWWWWWWWWWC

SEQ ID NO: 90 - protein, 20 aa, Artificial Sequence
  Synthetic construct
RRRRRRRRRRWWWWWWWWWW

SEQ ID NO: 91 - protein, 10 aa, Artificial Sequence
  Synthetic construct
KRRRRWWWWE

SEQ ID NO: 92 - protein, 11 aa, Artificial Sequence
  Synthetic construct
RRRRRKWWWWW

SEQ ID NO: 93 - protein, 13 aa, Artificial Sequence
  Synthetic construct
RRRRRRKWWWWWW

SEQ ID NO: 94 - protein, 12 aa, Artificial Sequence
  Synthetic construct
CRWRWRWRWRWC

SEQ ID NO: 95 - protein, 10 aa, Artificial Sequence
  Synthetic construct
RRRRCWWWWC

SEQ ID NO: 96 - protein, 10 aa, Artificial Sequence
  Synthetic construct
WWWWCRRRRC

SEQ ID NO: 97 - protein, 11 aa, Artificial Sequence
  Synthetic construct
WWCRRRRRCWW

SEQ ID NO: 98 - protein, 11 aa, Artificial Sequence
  Synthetic construct
WWWCRRRRRCW

SEQ ID NO: 99 - protein, 15 aa, Artificial Sequence
  Synthetic construct
WWWWCRRRRRCWWWW

SEQ ID NO: 100 - protein, 10 aa, Artificial Sequence
  Synthetic construct
CWRWRWRWRC

SEQ ID NO: 101 - protein, 12 aa, Artificial Sequence
  Synthetic construct
CWRWRWRWRWRC

SEQ ID NO: 102 - protein, 10 aa, Artificial Sequence
  Synthetic construct
CWRWRWRWRC

(19) United States
(12) Patent Application Publication
(10) Pub. No.: US 2023/0000999 A1
(43) Pub. Date: Jan. 5, 2023

(54) COMPOSITIONS AND METHODS FOR NUCLEIC ACID DELIVERY

(71) Applicants: Keykavous PARANG, Irvine, CA (US); Hamidreza MONTAZERI ALIABADI, Santa Margarita, CA (US); Dindyal MANDAL, Lake Forest, CA (US); Eman MOHAMMED, Menoufia (EG); Saghar MOZAFFARI, Irvine, CA (US); Sandeep LOHAN, Lake Forest, CA (US); Rakesh TIWARI, Irvine, CA (US); Ryley HALL, Tacoma, WA (US); AJK BIOPHARMACEUTICAL, LLC, Irvine, CA (US)

(72) Inventors: Keykavous PARANG, Irvine, CA (US); Hamidreza MONTAZERI ALIABADI, Santa Margarita, CA (US); Dindyal MANDAL, Lake Forest, CA (US); Eman MOHAMMED, Menoufia (EG); Saghar MOZAFFARI, Irvine, CA (US); Sandeep LOHAN, Lake Forest, CA (US); Rakesh TIWARI, Irvine, CA (US); Ryley HALL, Tacoma, WA (US)

(21) Appl. No.: 17/788,954

(22) PCT Filed: Dec. 24, 2020
(86) PCT No.: PCT/US2020/067036

§ 371 Date: Jun. 24, 2022

Related U.S. Application Data
(60) Provisional application No. 63/013,843, filed on Apr. 22, 2020.
(60) Provisional application No. 62/953,365, filed on Dec. 24, 2019.

(51) Int. Cl. A61K 47/64; C07K 7/08; C07K 7/06; C07K 7/64; A61K 48/00; A61K 47/69; C12N 15/113; C12N 15/11; A61P 35/00; A61K 8/64; A61Q 19/00; A61K 8/60; A61K 47/60

(52) U.S. Cl. CPC A61K 47/6455; C07K 7/08; C07K 7/06; C07K 7/64; A61K 48/0033; A61K 47/6923; A61K 47/6929; C12N 15/113; C12N 15/11; A61P 35/00; A61K 8/64; A61Q 19/00; A61K 8/606; A61K 47/60; C12N 2310/3513; C12N 2310/14; C12N 2310/20; C12N 2320/32

(57) ABSTRACT

Peptide-based systems containing hydrophobic amino acids (e.g., tryptophan), charged amino acids (e.g., arginine), and/or sulfur-containing amino acids (e.g., cysteine), which can be used either alone or in combination with nanoparticles (e.g., gold or silver nanoparticles) for siRNA delivery into living cells are disclosed.

### CROSS REFERENCE TO RELATED APPLICATIONS

This application claims the benefit of priority to U.S. Provisional Application No. 62/953,365, filed Dec. 24. 2019, and No. 63/013,843, filed Apr. 22, 2020, the disclosures of which are incorporated by reference herein in their entireties.

### FIELD

The disclosed subject matter relates to delivery of nucleic acids (DNA, plasmids, oligos, small interfering RNAs [siRNA], small hairpin RNA [shRNA], microRNAs [miRNA], Clustered Regularly Interspaced Short Palindromic Repeats [CRISPR]) using a peptide-based delivery system to living cells.

### BACKGROUND

Nucleic acid delivery is an approach to suppress (or enhance) the expression of a protein temporarily or permanently. The suppression of protein expression (also known as silencing) is performed by delivering double-stranded siRNAs, shRNA, miRNA, or CRISPR/Cas9. RNA interference (RNAi) is an approach to suppress (or stop) the expression of a protein temporarily or permanently. The double stranded siRNA contains a passenger and a guide strand. The guide strand is incorporated into the RNA-induced silencing complex (RISC), while the passenger strand is degraded. The guide strand acts as a complementary sequence to the messenger RNA, and therefore, binds to the targeted mRNA, which triggers the Argonaute 2 (an essential catalytic protein in RISC) to cleavage the mRNA to small pieces, which will be degraded rapidly by RNases. This process is known as the post-transcriptional gene silencing (Akinc et al., Targeted delivery of RNAi therapeutics with endogenous and exogenous ligand-based mechanisms. Mol Ther, 2010, 18(7):1357-64), This complex intervenes in the degradation process of the siRNA sense strand and utilizes the preserved anti-sense strand to identify the complement sequences of mRNA.

Clinical evaluation of nucleic acids has been challenging due to their limited cellular uptake (because of large size and negatively charged phosphate groups in their structure), off-target effects, and rapid enzymatic degradation in vivo. Even though double stranded nucleotides are generally more stable, siRNA is still extremely susceptible to enzymatic degradation in biological settings (Layzer et al., In vivo activity of nuclease-resistant siRNAs. RNA, 2004, 10(5):765-71). Also, due to its small size, siRNA is readily eliminated from the body through glomerular filtration. In many animal studies, renal clearance of siRNA correlated to higher uptake activities fan de Water et al., Intravenously administered short interfering RNA accumulates in the kidney and selectively suppresses gene function in renal proximal tubules, Drug Metab Dispos, 2006, 34(8): 1:393 -7). Clearance occurs in the kidney where the phagocytes aggregate the siRNA by using the serum proteins. The nuclease degradation of siRNA can occur by the reticuloendothelial system (RES). Finally, the negatively charged and hydrophilic small RNA structures have minimal interaction with the cell membrane (which is also negatively charged), and therefore, the cellular internalization is negligible.

Several delivery systems have been evaluated to improve the efficiency of siRNA, which include polymers, lipids, liposomes, and carbon nanotubes (Liu et al., SiRNA delivery systems based on neutral cross-linked dendrimers. Bioconjug Chem, 2012, 23(2):174-83; Chabot et al., Targeted electro-delivery of oligonucleotides for RNA interference: siRNA and antimiR. Adv Drug Deliv Rev, 2015, 81:161-8; de Fougerolles et al., Interfering with disease: a progress report on siRNA-based therapeutics. NatRev Drug Discov, 2007, 6(6):443-53; Golzio et al., In vivo gene silencing in solid tumors by targeted electrically mediated siRNA delivery. Gene Ther, 2007, 14(9):752-9; Oh et al., siRNA delivery systems for cancer treatment. Adv Drug Deliv Rev, 2009, 61(10):850-62), However, the majority of the delivery systems exhibited significant cytotoxicity and/or demonstrated limited in vivo and clinical potential. For example, poly(ethyleneimine) (PEI) is a commonly used polymeric carrier for siRNA delivery. The use of PEI facilitates the endosomal release of siRNA in the cellular cytoplasm. However, PEI induces toxicity contributes to low transfection efficiency (Guo et al., Engineering RNA for targeted siRNA delivery and medical application. Adv Drug Deliv Rev, 2010, 62(6):650-66).

Because of the great therapeutic potential of nucleic acids, developing efficient delivery systems is highly demanded in clinical investigations. FDA recently approved Onpattro™ (siRNA/lipid complex) for the treatment of hATTR amyloidosis. The delivery systems should be biodegradable to minimize the toxicity in the cytoplasm. Cell-penetrating peptides (CPPs) have drawn significant attention in the drug delivery field. CPPS take advantage of their unpatrolled properties like biocompatibility and cell penetration ability to pass the plasma membrane or through endocytic pathways. The intrinsic property of CPPs to deliver therapeutic molecules (nucleic acids, drugs, imaging agents) to cells and tissues in a nontoxic manner has indicated that they may be beneficial for enhancing the cellular delivery of drugs and diagnostic agents with limited cellular uptake. The modification of CPPs is required to act as delivery vectors for nucleic acid delivery to enhance interaction with the nucleic acid and to minimize the degradation and mediate the efficient cellular uptake. Often, the modification of CPPs can occur by attaching hydrophilic and hydrophobic lipid tails via linker residues. Attachment of lipids to the CPPs has been shown to facilitate membrane interactions and improve cellular uptake (Hafez et al., On the mechanism whereby cationic lipids promote intracellular delivery of poly nucleic acids. Gene Ther, 2001, 8(15):1188-96).

Cyclic peptides containing alternate tryptophan and arginine residues [WR]₅ (SEQ ID NO.: 1) and [WR]₄(SEQ ID NO.: 2) (FIG. 1) and the corresponding peptide-capped gold nanoparticles were found to act as efficient molecular transporters of siRNA in human cervix adenocarcinoma (HeLa) cells (Nasrolahi Shirazi et al., Cyclic peptide-capped gold nanoparticles for enhanced siRNA delivery. Molecules 2014, 19:13319-13331). Flow cytometry studies showed that [SVR]₅ (SEQ ID NO.: 1) and [WR]₅-capped gold nanoparticles improved the intracellular uptake of siRNA versus siRNA alone. It was also found that both delivery platforms were less toxic when compared with lipofectamine. Fluorescence microscopy data confirmed the localization of fluorescence-labeled siRNA in the presence of [WR]₅ (SEQ ID NO.: 1) and [WR]₅-capped gold nanoparticles. Thus, the positively charged residues on the peptide could have electrostatic interactions with negatively charged phosphate residues in the phospholipid bilayer and siRNA. Furthermore, the hydrophobic tryptophan groups could interact with hydrophobic residues in the lipid membrane. The efficacy of fatty acid-conjugated linear and cyclic arginine/lysine peptides in delivering siRNA to breast cancer cells and the efficacy of protein silencing via this approach was also shown (Do et al., Difatty Acyl-conjugated linear and cyclic peptides for siRNA Delivery. ACS Omega 2017, 2:6939-6957). A variety of linear and cyclic peptides were synthesized with various fatty acyl conjugations with different lengths (from C2 to C18) and degree of saturation (FIGS. 2 and 3). Most recently, the potential of [WR]₅(SEQ ID NO.: 1) for siRNA delivery was further confirmed (Mozaffari et al., Amphiphilic peptides for efficient siRNA delivery, Polymers 2019, 11(4):703).

The delivery of siRNA complexes has been improved by targeting surface biomarker exclusively overexpressed on the disease's cells. One such example is targeting the 60 vβ3 integrin receptor in the angiogenic blood cells and tumor using linear or cyclic RGD peptide to improve the delivery of siRNA to the cancerous cells (Fu et al., RGD peptide-based non-viral gene delivery vectors targeting integrin αv β3 for cancer therapy. J Drug Target 2019, 27:1-11).

Thus, there is still a need for compositions and methods that provide efficient and non-toxic delivery of siRNAs to living cells. The compositions and methods disclosed herein addresses these and other needs.

### SUMMARY

Disclosed herein is a peptide-based systems containing hydrophobic amino acids (e.g., tryptophan), charged amino acids (e.g., arginine), and/or sulfur-containing amino acids (e.g., cysteine), which can be used either alone or in combination with nanoparticles (e.g., gold or silver nanoparticles) for siRNA delivery into living cells. Some of the peptides contain a disulfide bridge for cyclization and two chains of hydrophobic tryptophan residues. The amphiphilic cyclic and linear peptides and the corresponding peptide-capped gold nanoparticles were compared for siRNA delivery (Nasrolahi Shirazi et al., 2014). The rationale of this design is based on the data that a cyclic peptide [WR]₅ (SEQ ID NO.: 1) containing alternating tryptophan (W) and arginine (R) residues (e.g., WRWR ((SEQ ID NO.: 3)) significantly enhanced the cellular uptake of negatively charged phosphopeptides and siRNA (Nasrolahi Shirazi et al., 2014).

Peptide carriers containing tryptophan, arginine, and/or cysteine for binding with siRNA, protection against enzymatic degradation, cellular internalization, and silencing are disclosed. The peptides and their corresponding gold nanoparticles were used in different ratios with siRNA for comparative studies in cancer cells. Furthermore, the effect of hydrophobic modification of the formulation of each peptide/siRNA complex was also evaluated. The size and surface electrical charge were evaluated for the complexes formed via ionic interaction between the peptides and siRNA to confirm complex formation and neutralization of siRNA negative charge and to evaluate the physical characteristics of the complex. The binding affinity of the peptides to siRNA were analyzed to determine the effect of peptide and the size of the conjugate on the inter-ionic interaction. The ability of the peptides in protecting siRNA against early enzymatic degradation was investigated by exposing the complexes to fetal bovine serum, and the toxicity of the peptides was explored in different human cancer cell lines. Finally, the efficiency of the peptides in internalizing siRNA into different human cancer cells was studied, which was confirmed by silencing efficiency for specific proteins.

Although embodiments that incorporate siRNAs are described at length, peptide and/or peptide-capped nanoparticles are useful for the delivery of other species of nucleic acid (e.g., ssDNA, dsDNA, DNA:RNA hybrids, etc.) as well as other negatively charged polymer species. As such, compositions and methods disclosed herein can be utilized in gene silencing, transient expression, and transient or permanent genetic modification in living cells,

Delivery of short interfering RNAs (siRNAs) remains a major challenge in the development of RNA interference therapeutics. siRNA is a class of double-stranded RNA molecules that are about 20-25 base pairs. An optimal siRNA delivery system for in vivo use must facilitate cellular uptake and enhance its activity. Second, it must direct the siRNA toward the target tissue effectively.

Compositions disclosed herein can include linear, cyclic, and/or hybrid linear/cyclic peptides containing natural or non-natural positively-charged amino acids, hydrophobic residues, and/or cysteine residues for use as siRNA delivery systems. Disclosed herein are four classes of peptides, which can include the addition of cysteine residues that can form disulfide bonds, modifications, such as the substitution of L-amino acid with D-amino acids to avoid proteolytic enzymes, the substitution of positively charge arginine or hydrophobic residues with non-natural amino acids, have significantly higher stability, loading efficiency, less toxicity, and effective silencing. Furthermore, the disulfide bridge can be reduced in the presence of glutathione, possibly releasing encapsulated siRNA in the peptide/siRNA complex.

Various objects, features, aspects, and advantages of the subject matter disclosed herein will become more apparent from the following detailed description of preferred embodiments, along with the accompanying drawing figures in which like numerals represent like components.

One should appreciate that the disclosed techniques provide many advantageous technical effects, including the efficient and direct introduction of nucleic acids (such as siRNA) into living cells.

### BRIEF DESCRIPTION OF THE FIGURES

FIG. 1. Cyclic peptides containing alternative tryptophan and arginine residues, [WR]₄ (SEQ ID NO.: 2) and [WR]₅ (SEQ ID NO.: 1) (Nasrolahi Shirazi et al., 2014; Mozaffari et al., 2019).

FIG. 2. Chemical structures of linear peptide-fatty acid conjugates reported by us (Do et at, 2017),

FIG. 3. Fatty acyl conjugates of cyclic peptides previously reported by us (Do et al., 2017),

FIG. 4, Chemical structures of class I peptides (Class I: cyclic and linear peptides containing alternative tryptophan and arginine residues and two cysteines).

FIG. 5. Chemical structures of class 2 peptides: cyclic peptides containing tryptophan residues in one side and arginine residues on another side and two cysteines).

FIG. 6. Chemical structures of class 3 peptides: linear and cyclic-linear peptides containing two chains of hydrophobic tryptophan residues attached to polyarginine linear peptides or cyclic peptides containing arginine residues through cysteine residues.

FIG. 7. Chemical structures of class 3 peptides: cyclic-linear peptides containing two chains of hydrophobic tryptophan residues attached to polyarginine linear peptides or cyclic peptides containing arginine residues through lysine residues.

FIG. 8. Synthesis of linear and cyclic peptide cyclized through a disulfide bridge.

FIG. 9. A representative example of synthesis of the cyclic peptide ([CR₅W₅C] (SEQ ID NO.: 4)).

FIG. 10. The formation of peptide-capped gold nanoparticles (Peptide-AuNPs) from the reaction of peptides with HAuCl₄.

FIG. 11. the formation of gold nanoparticles by compounds in class 4 ([W₄-β-Ala-R₄-β-Ala] (SEQ ID NO.: 5) and (W₄-βAla-R₄-βAla) (SEQ ID NO.: 6). The synthesis of gold nanoparticles is monitored by UV-vis spectroscopy. The appearance of the absorbance band at 530 nm confirms the synthesis of gold nanoparticles.

FIG. 12. The formation of gold nanoparticles by compounds in class 4 ([W₄-(βAla)₂-R₄-(βAla)₂] (SEQ ID NO.: 7) and W₄-(βAla)₂-R₄-(βAla)₂(SEQ ID NO.: 8)),

FIG. 13. Overview of the synthetic scheme for the synthesis of pegylated cell-penetrating peptide (PEG-CPP), Pegylated targeting peptide (PEG-TP) composed of cyclic RGD, and final conjugate (TP-PEG-CPP) (targeting peptide=TP=RGDdFK (SEQ ID NO.: 9)).

FIG. 14. Serum stability of siRNA after complex formation. Representative image of gel electrophoresis and bar graph summarizing the percentage of intact siRNA after 24 h exposure to serum (25%) at 37° C.

FIG. 15. The serum stability of siRNA complexed with lead peptides as compared to “naked” siRNA.

FIG. 16. A graph showing cytotoxicity of peptide/siRNA complexes in MBA-MB-231 breast cancer cells. Neither of the peptides showed significant toxicity in the range of concentration or N/P ratio evaluated.

FIG. 17. Cell viability of the peptides and the peptide/siRNA complexes in MDA-MB-231 cells after 48 h incubation.

FIG. 18. A graph showing cell uptake of led siRNA/peptide complexes at an N/P ratio of 20 compared to Lipofectamine™ 2000 (Lipo.).

FIG. 19. Cellular uptake of FAM-siRNA in MDA-MB-231 cells. The internalization of FAM-labeled siRNA into MDA-MB-231 after 24 h. Right panels in each set of peptides are presented as the percentage of the cells positive for siRNA uptake. The average of the fluorescence in the cell population is shown in the left panel. The cells were transfected by peptide/siRNA complex at an N/P ratio of 40. Data are presented as mean, n=3, and error bars represent standard deviation.

FIG. 20. siRNA uptake using gold nanoparticles generated by linear peptide R₄-βAla-W₄-β-Ala (SEQ ID NO.: 10).

FIG. 21. siRNA uptake using gold nanoparticles generated by linear peptide R₄-(βAla)₂-W₄-(βAla)₂ ((SEQ ID NO.: 11)

FIG. 22. A group of micrographs showing uptake of siRNA/'peptide complexes (N/P 20) by confocal microscopy.

FIG. 23. Silencing of Stat3 with peptides (Western blot analysis).

FIG. 24. Silencing of Stat3 by peptides.

FIG. 25. Transfection with gold nanoparticles generated with linear R₄w₄ (SEQ ID NO.: 12) and R₅W₅(SEQ ID NO.: 13) in MDA-MB-231 cells.

FIG. 26. Transfection with gold nanoparticles generated with linear R₄-β-Ala-W₄-β-Ala (SEQ ID NO.: 10) and R₄-βAla)₂-W₄-(β-Ala)₂ (SEQ ID NO.: 11) in MDA-MB-231 cells.

FIG. 27. Silencing of Stat3 with peptides (Western blot analysis).

FIG. 28. Silencing of Src with peptides (Western blot analysis).

FIG. 29. Transfection of HEK293 cells with GFP plasmid using Lipofectamine (positive control) and selected peptides. Images were prepared using fluorescent microscope 48 hours after transfection. The toxicity of lipofectamine is observed as detachment of the cells, which creates aggregates of floating cells in the media.

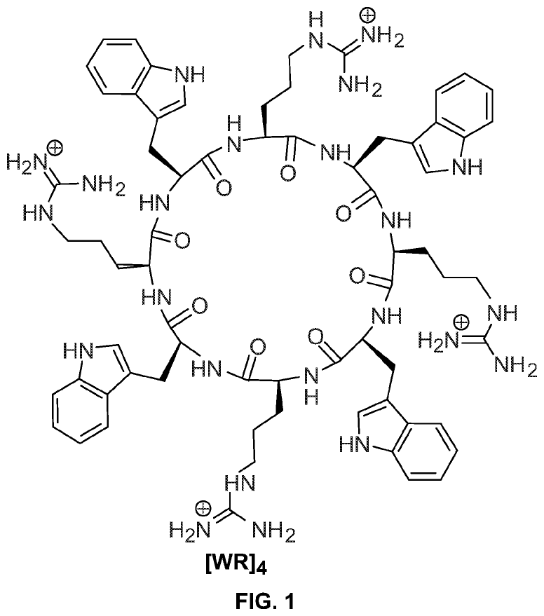

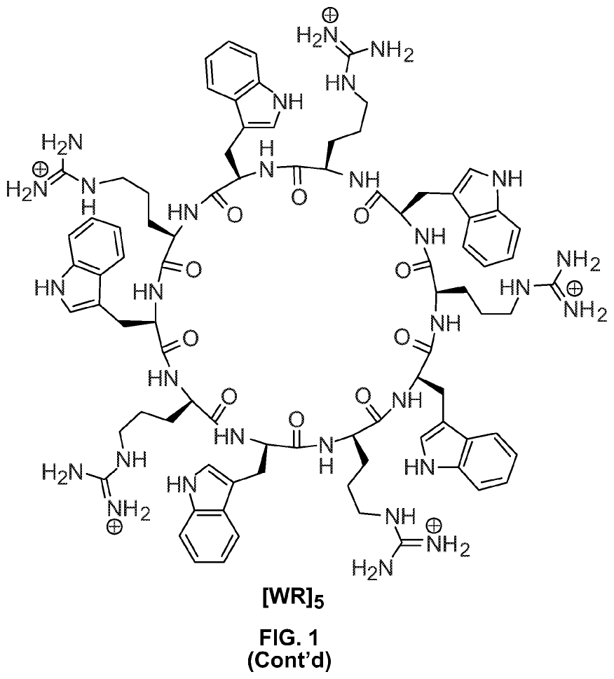

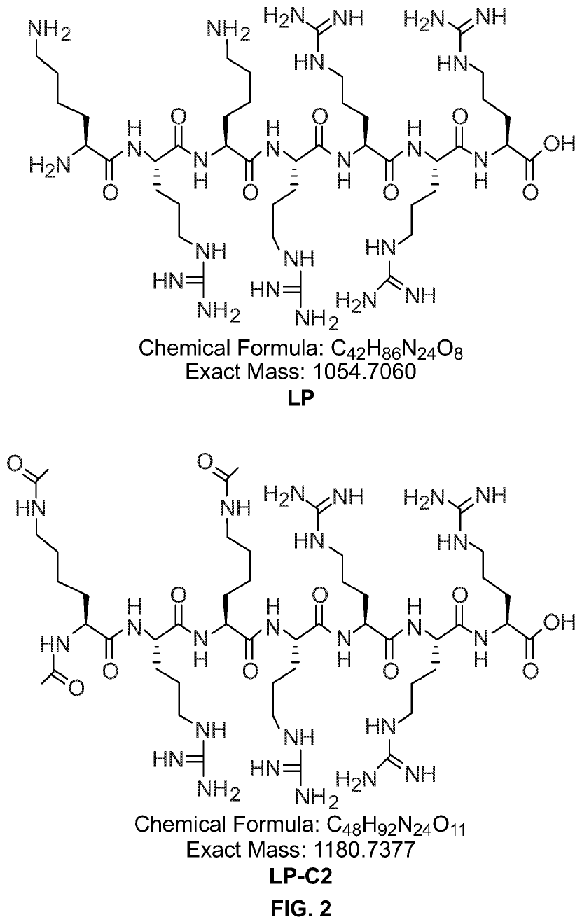

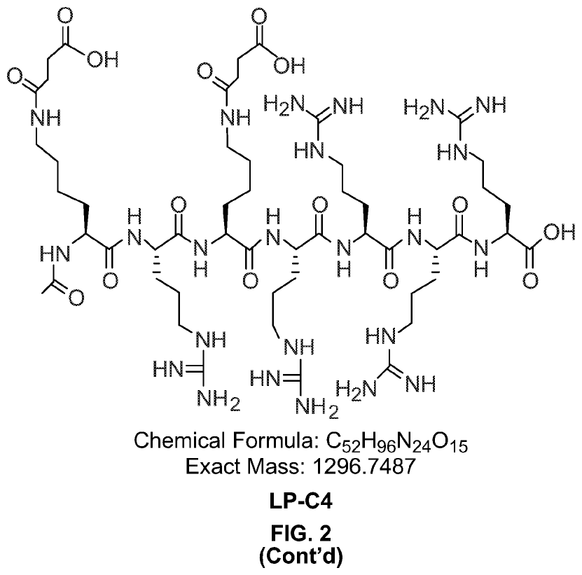

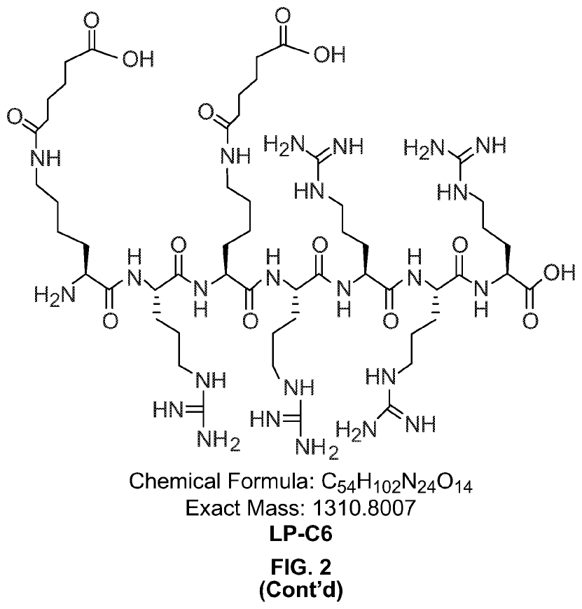

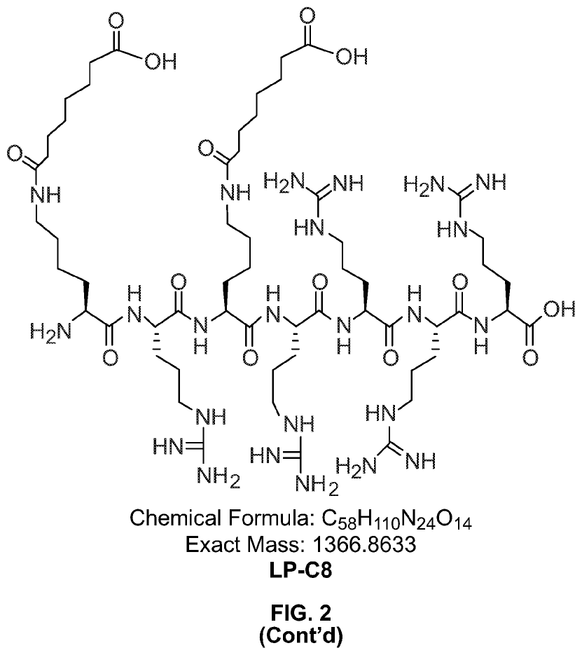

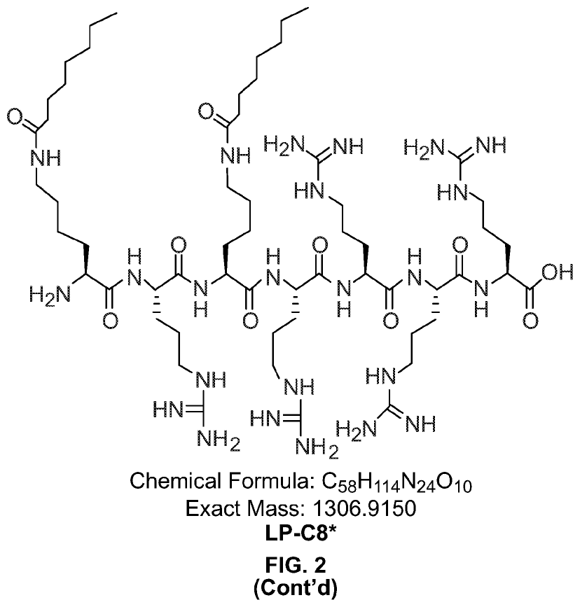

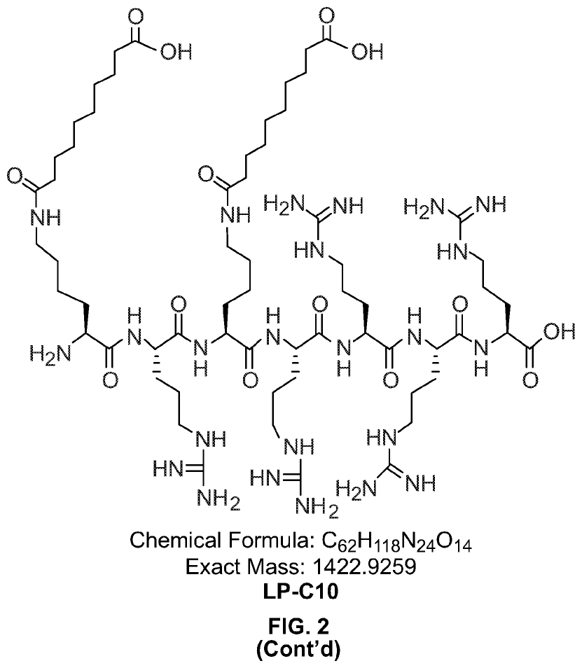

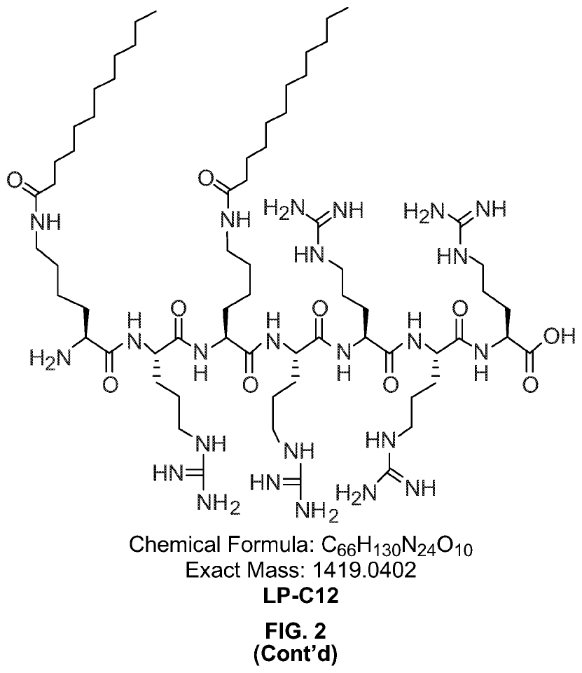

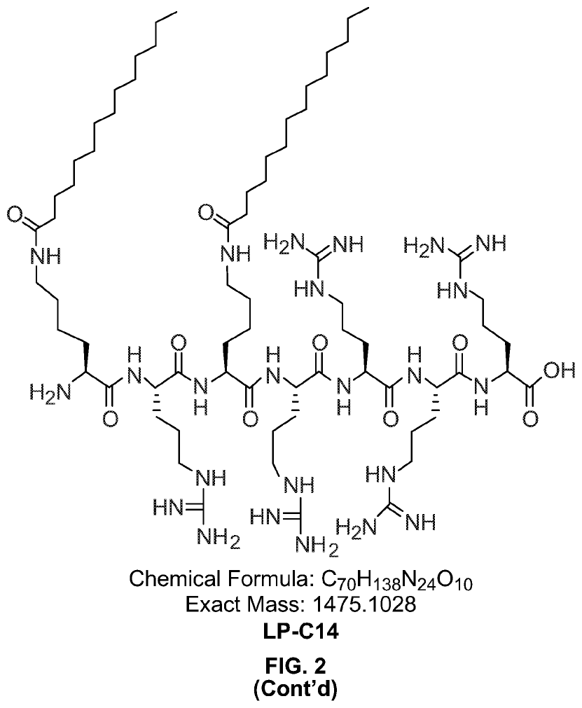

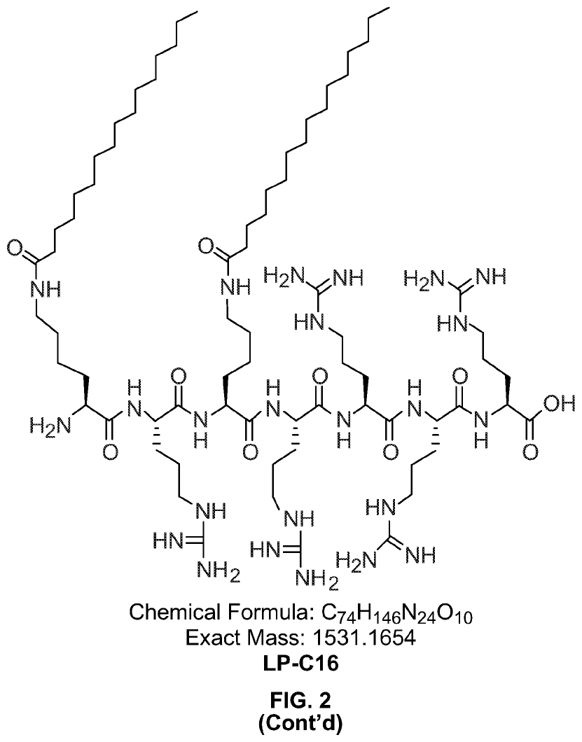

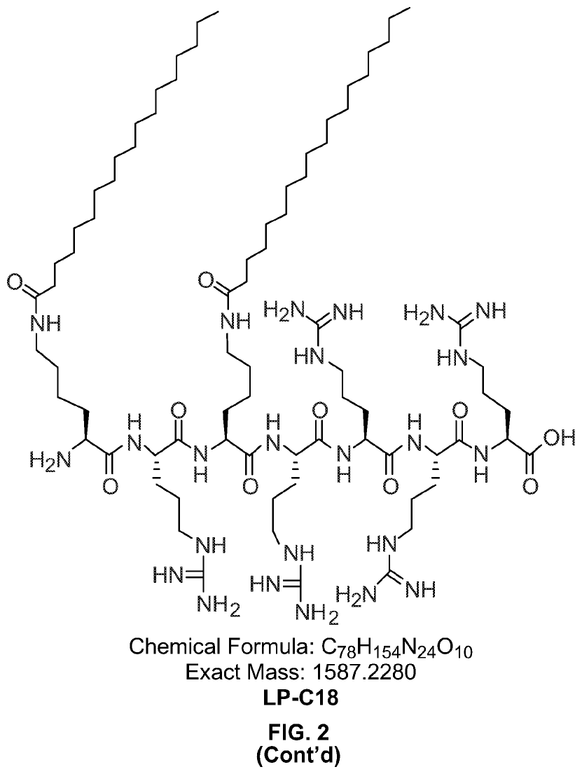

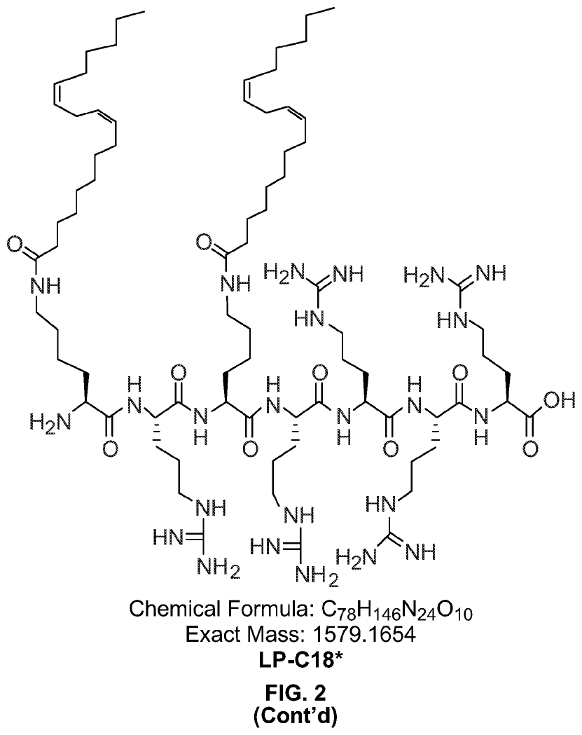

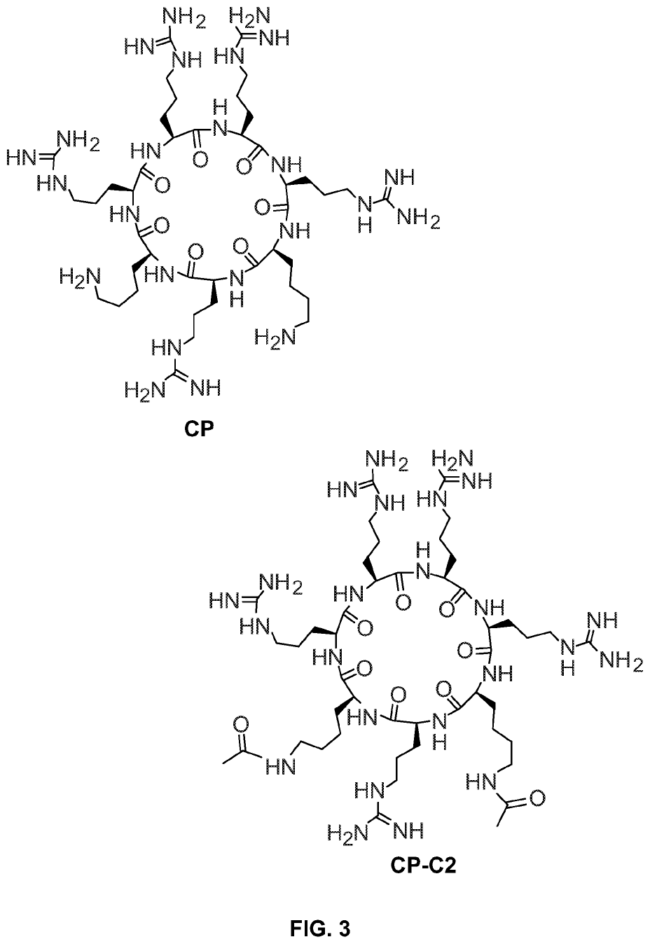

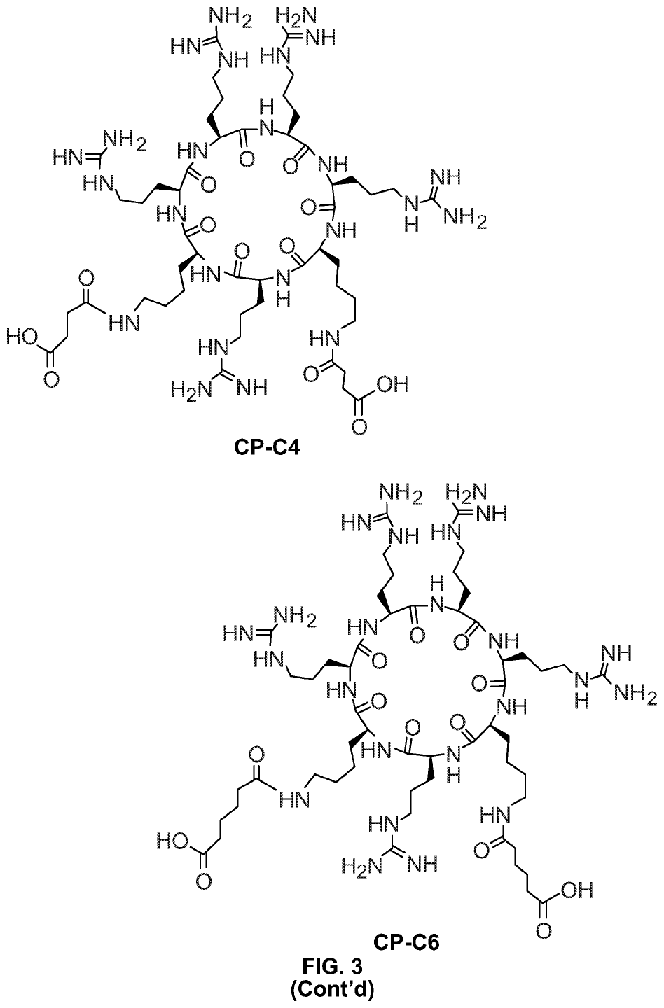

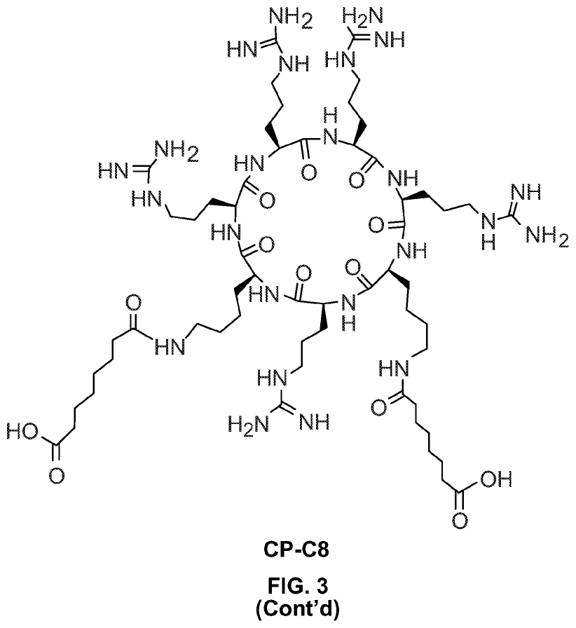

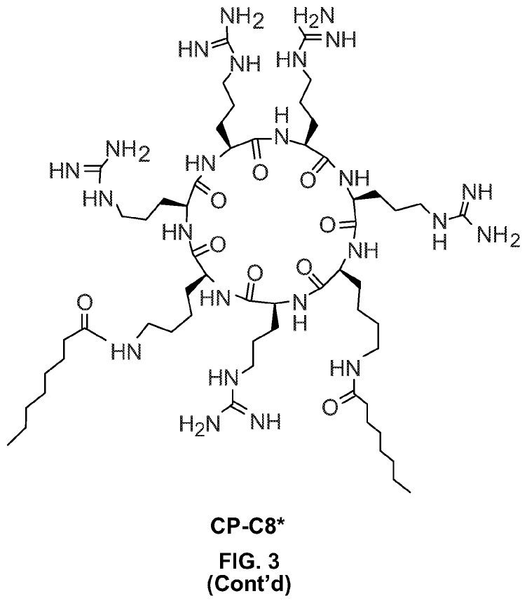

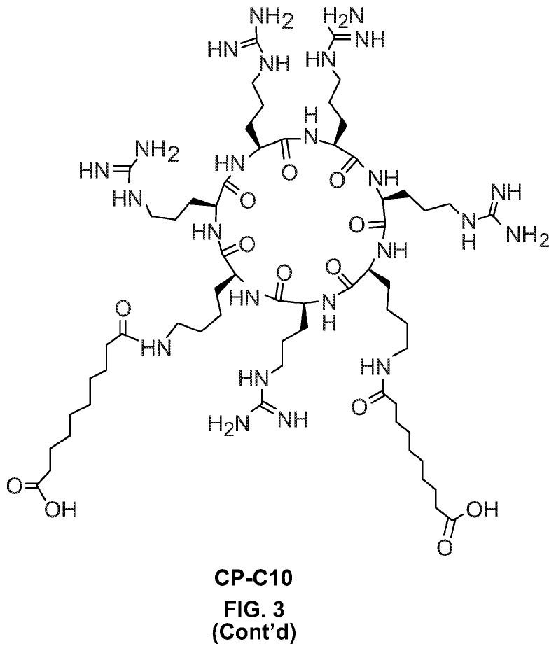

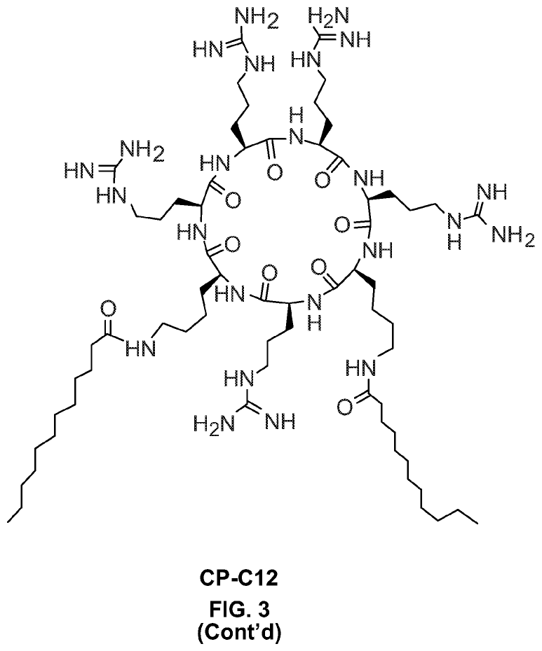

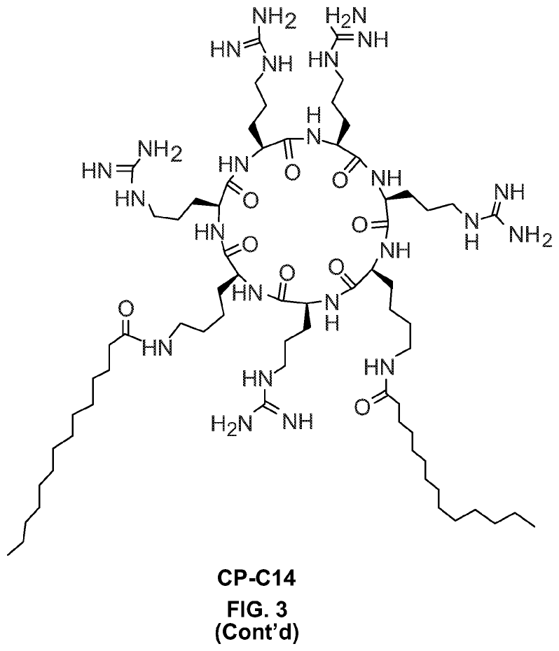

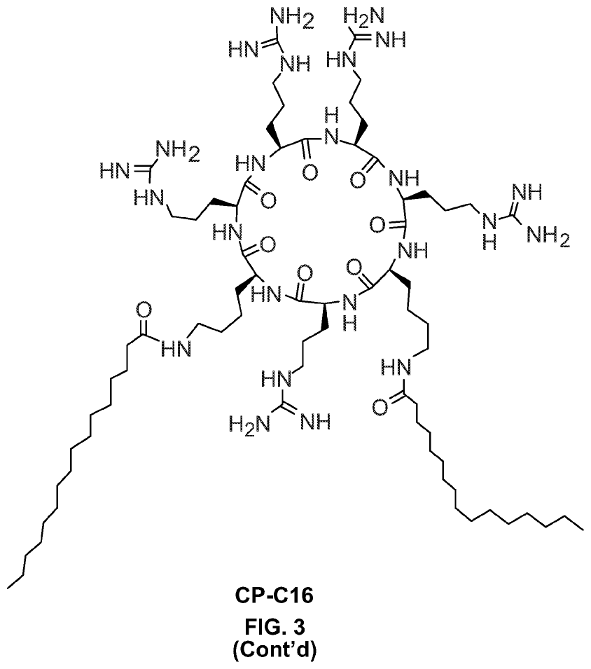

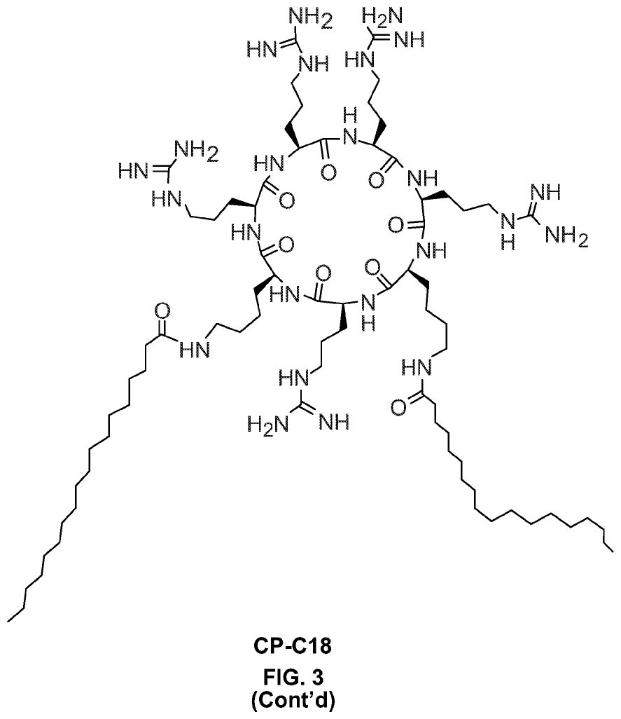

### DETAILED DESCRIPTION

Although each embodiment represents a single combination of inventive elements, the disclosed subject matter is considered to include all possible combinations of the disclosed elements. Thus, if one embodiment comprises elements A, B, and C, and a second embodiment comprises elements B and D, then other remaining combinations of A, B, C, or D, are considered disclosed and contemplated even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable, The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Throughout the description and claims of this specification the word “comprise” and other forms of the word, such as “comprising” and “comprises,” means including but not limited to, and is not intended to exclude, for example, other additives, components, integers, or steps.

As used herein, the terms “optional” or “optionally” means that the subsequently described event or circumstance can or cannot occur and that the description includes instances where said event or circumstance occurs and instances where it does not.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The term “hybrid peptide” is used to represent a peptide, which consists of a combination of linear and cyclic peptides. The peptide library was screened for their biophysiochemical properties, such as binding affinity for siRNA, particle size and surface charges of peptide/siRNA complex, and the stability of the complex in the presence of serum, and the siRNA release profile to establish a structure-function relationship.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified, thus fulfilling the written description of all Markush groups used in the appended claims.

Compositions

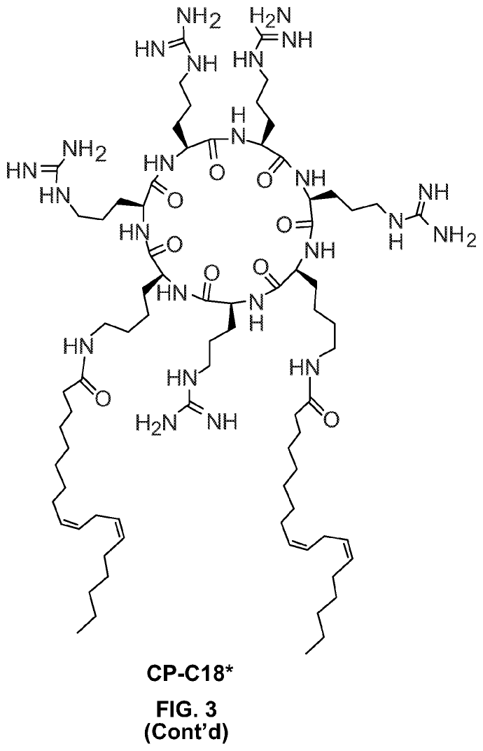

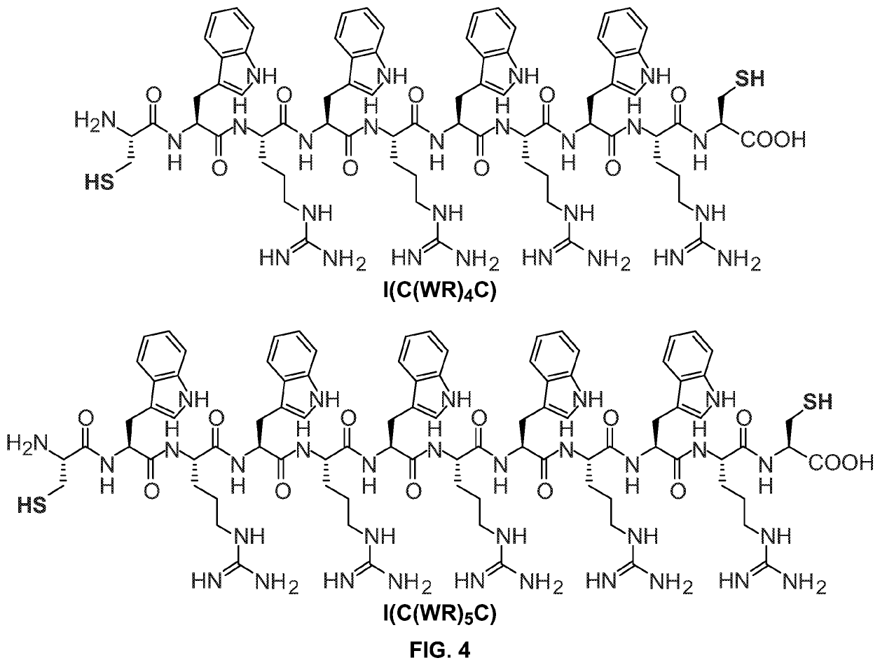

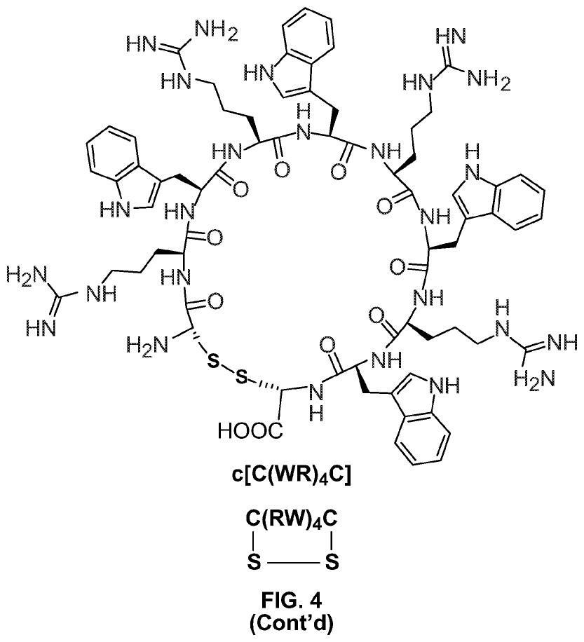

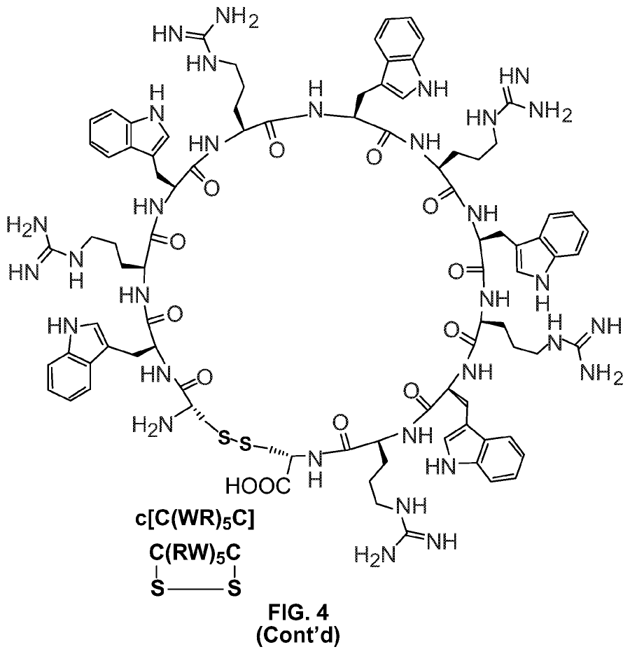

The first class of peptides contains positively charged amino acids (e.g.) arginine and hydrophobic amino acids (e.g., tryptophan residues) in an alternating fashion, where the peptide is cyclized through a disulfide linkage of two cysteine residues positioned at the IV- and C-termini of a linear precursor peptide (FIG. 4), It should be appreciated that such an arrangement introduces a disulfide bond into the primary ring structure of the cyclic peptide.

Class 1 includes [C(RW)ₙC]; wherein n=1-10. The increase in positive charge density and hydrophobicity will enhance optimal interaction with siRNA by modulating R and W residues (alternate or R on one side and W on opposite side) between the disulfide bridges. To demonstrate the role of the disulfide bridge in the release of nucleic acid from the peptide/siRNA complexes, the linear and amide derivatives of peptides (replacement of both C amino acids with glutamic acid (E) and lysine (K) residues to form amide bridge) were also synthesized.

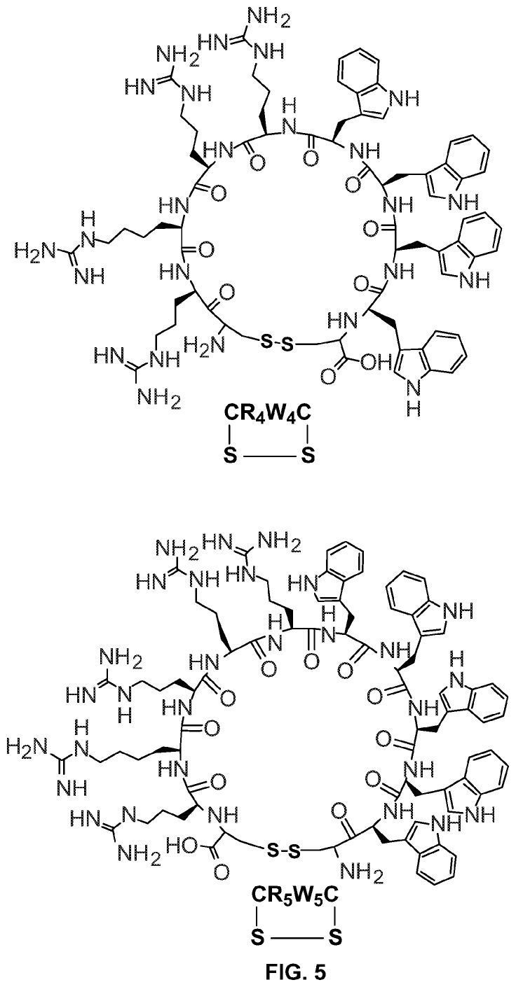

A second class of compounds (FIG. 5) includes cyclic peptides containing hydrophobic amino acids (e.g., tryptophan residues) in one block or segment and positively charged amino acids (e.g., arginine residues) in another block or segment, as well as two cysteines (for example, positioned at the N- and C-termini) that can be free or utilized for cyclization via disulfide formation. Class 2 includes peptides [CRₐW_{b}C]; wherein a=1-10, b=1-10. The number of arginine and tryptophan residues can range from 1 to 10. Alternatively, this class of peptides includes peptides [RₐW_{b}] composed of with R residues on one side and hydrophobic W residues on the other side, and the peptides are cyclized using N- to C-terminal cyclization ([RₐW_{b}] where a=1-10, b=1-10).

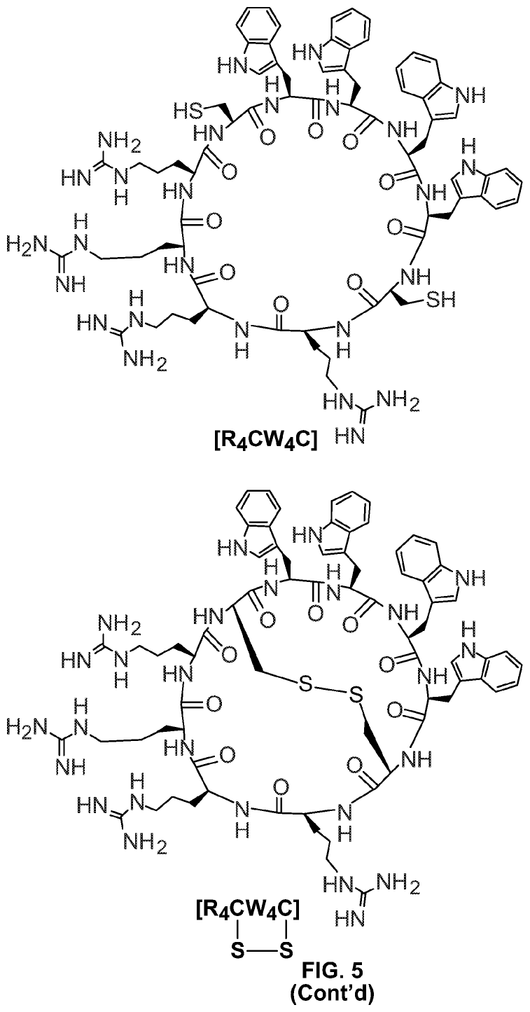

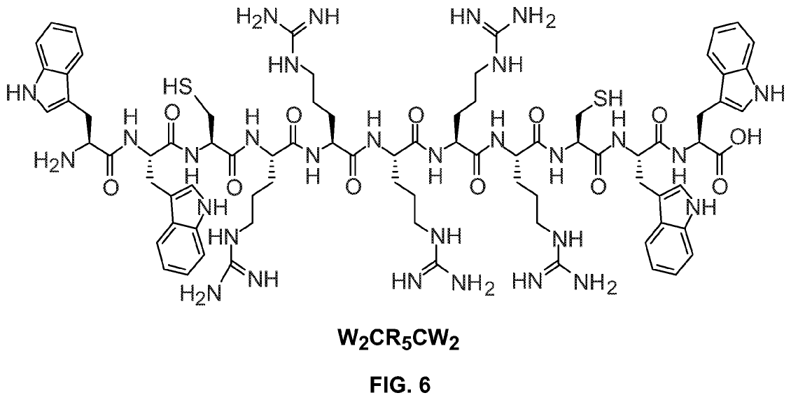

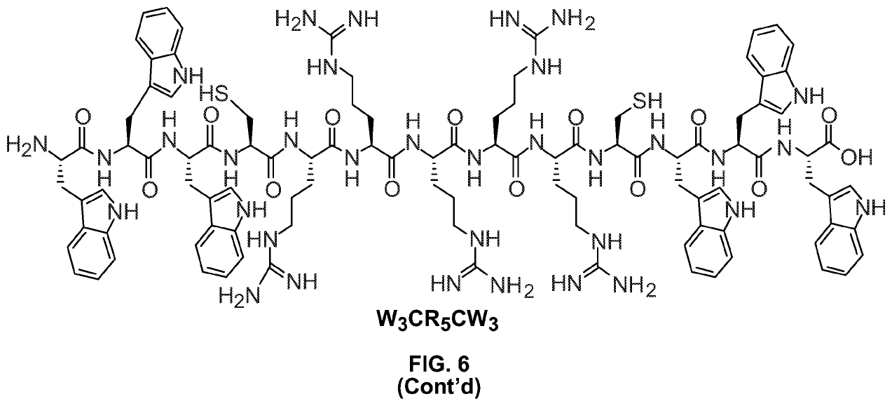

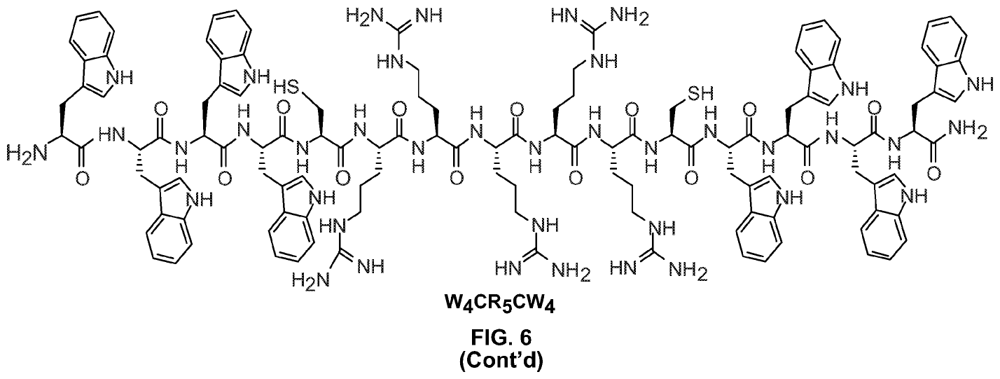

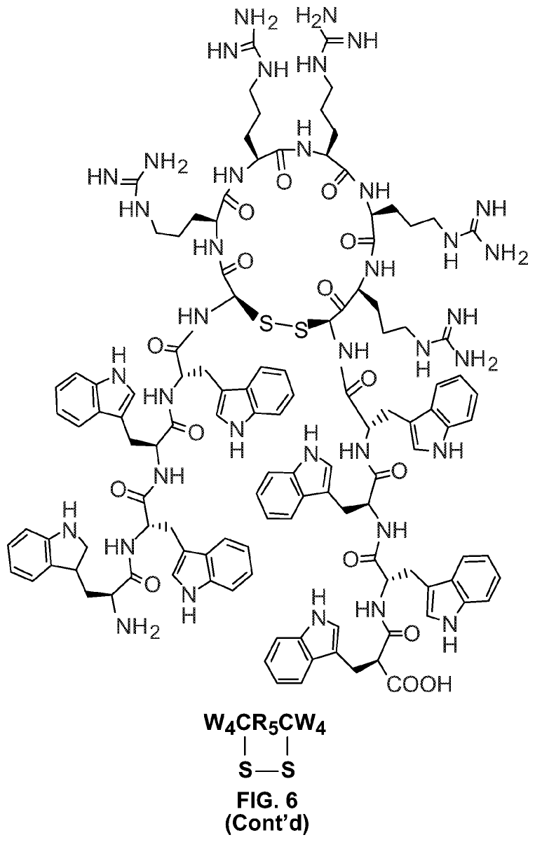

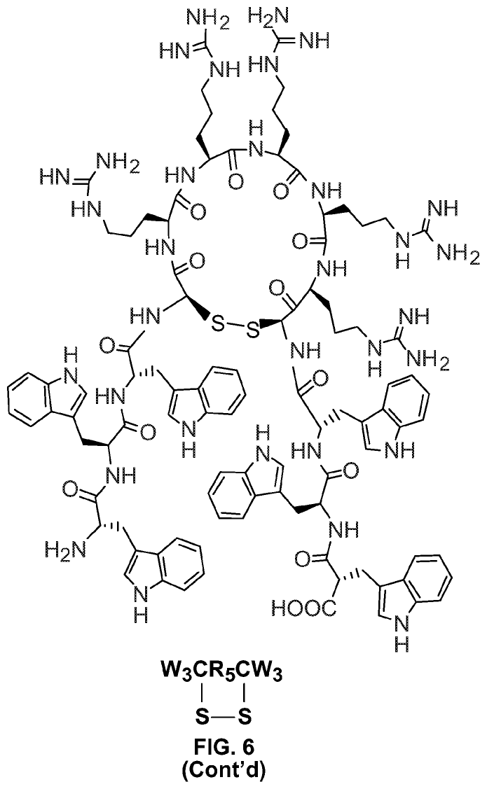

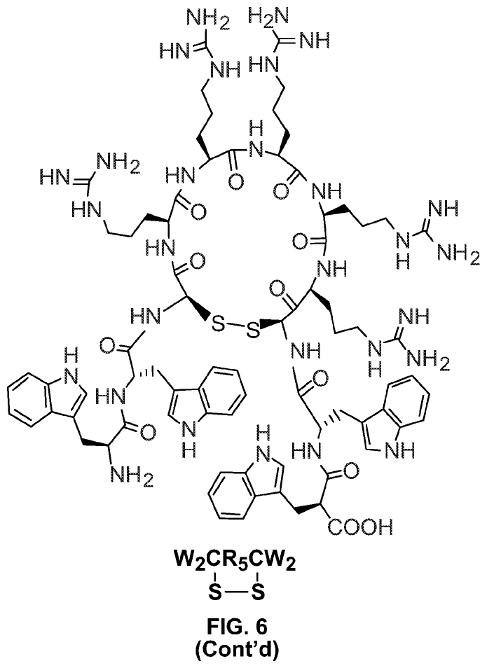

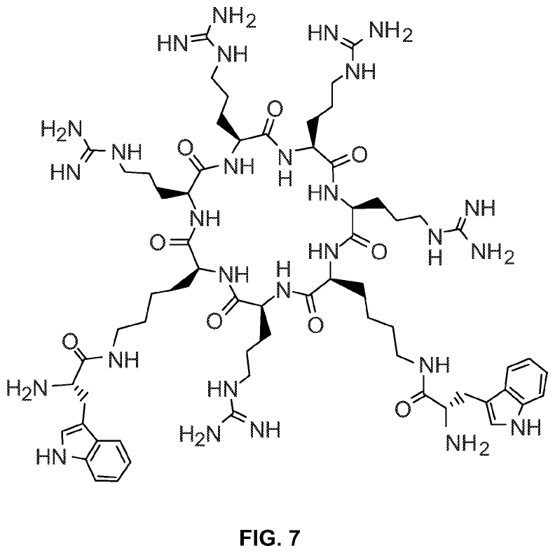

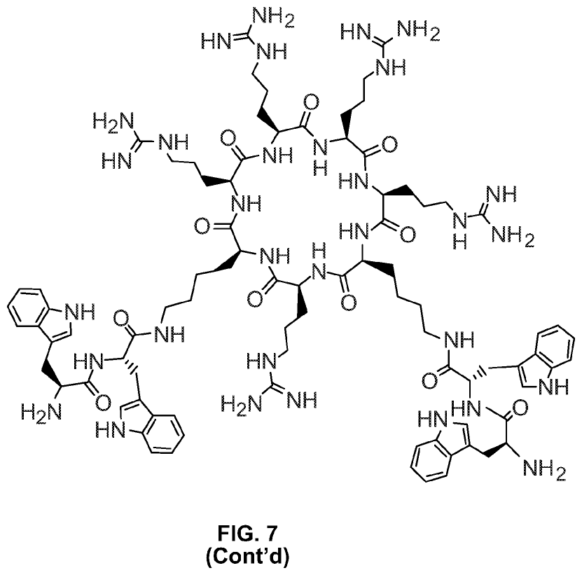

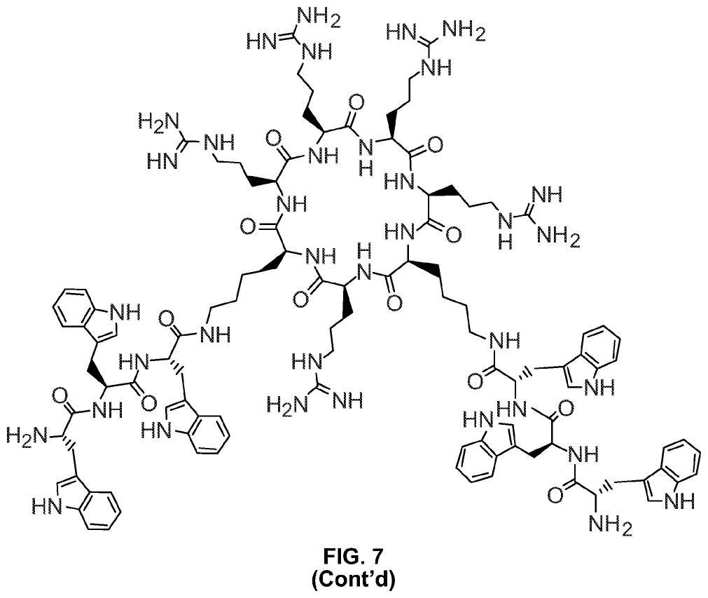

A third class of peptides (FIG. 6) includes linear peptides containing two linear chains of hydrophobic amino acids (e.g., tryptophan residues) attached to a linear poly-positively charged amino acid (e.g., polyarginine) segment to form linear peptides or hybrid cyclic-linear peptides containing cyclic poly-positively charged amino acid (e.g., polyarginine) cyclized through a disulfide linkage and linked to two linear chains of hydrophobic amino acid (e.g., tryptophan) residues. The third class of peptides also includes hybrid cyclic-linear peptides containing cyclic poly-positively charged amino acid (e.g., polyarginine) residues cyclized through an amide linkage and linked to two chains of hydrophobic amino acid (e.g., tryptophan) residues through lysine residues (FIG. 7).

This class of peptides Wₓ[CR_{y}C]Wₓ or (Rx[CW_{y}C]Rₓ where x=1-10, y=1-10) is based on the lead peptide W₄[CR₅C]W₄(SEQ ID NO.: 14). The optimal balance of hydrophobicity was achieved with optimizing linear chains of residues and R residues in the cyclic component in this class of peptides.

Alternatively, the corresponding linear peptides (W)ₙ(KRₙK)(W)ₙ and (W)ₙ(Rₙ)(W)ₙ can be included in this group. The number of positively charged (e.g., arginine) and hydrophobic (e.g., tryptophan) residues can range from 1 to 10.

The fourth class of compounds composed of linear and cyclic peptides containing positively-charged residues such as arginine and hydrophobic residues such as tryptophan separated from each other by beta alanine (W₄(β-alanine)ₓ-Rₙ-(β-alanine)ₓ where n=1-10, and x=1-5. Examples of compounds in this group are [W₄-βAla-R₄-βAla] (SEQ ID NO.: 5) and (W₄-βAla-R₄-βAla) ((SEQ ID NO.: 6)).

Fourth classes of compounds can be used alone for nucleic acid delivery or be used for the generation of peptide-capped gold nanoparticles that are used for nucleic acid delivery.

The peptides (see examples in Table 1) were synthesized using Fmoc/tBu solid-phase synthesis. In brief, the linear peptides were assembled on trityl resin. The amino acids in the sequence were conjugated using Fmoc-amino acid building blocks in the presence of 2-(1H-benzotriazole-1-yl)-1,1,3,3-tetramethyluronium hexafluorophosphate (HBTU), and diisopropylethylamine (DIPEA) in dimethylformamide (DMF). After each coupling, the Fmoc deprotection performed using 20% (v/v) piperidine in DMF. The progress of the reaction was monitored by matrix-assisted laser desorption/ionization (MALI) time-of-flight (TOF) analyzer. The resultant peptides were cleaved from the resin, followed by purification and lyophilization. The linear peptides containing cysteine residues were oxidized in the presence of 10% DMSO/Water to introduce disulfide bridges. The amide bridged derivative was synthesized by using orthogonal protecting K and E residues that are selectively deprotected on a solid support to form an amide bridge in the side chains followed by complete deprotection and purification.

TABLE 1
Representation of the composition of classes 1-4 peptides.

| | | | | | Class 4 |
|---|---|---|---|---|---|
| | Class 2 | Class 2 | Class 3 | Class 3 | [Wₙ(β₄-alanine)ₓ- |
| Class 1 | [CRₐW_{b}C] | [RₐW_{b}] | Wₓ[CR_{y}C]Wₓ | Rₓ[CW_{y}C]Rₓ | Rₙ(β-alanine)ₓ] |
| [C(RW)ₙC] | a = 1-10, | a = 1-10, | x = 1-10, | x = 1-10, | n = 1-10, and |
| n = 1-10 | b = 1-10 | b = 1-10 | y = 1-10 | y = 1-10 | x = 1-5 |
| [C(RW)₃C] | [CR₄W₄C] | [R₄W₄] | W₂[CR₅C]W₂ | R₂[CW₅C]R₂ | [W₅-βAla-R₅-βAla] |
| (SEQ ID | (SEQ ID | (SEQ ID | (SEQ ID | (SEQ ID | (SEQ ID |
| NO.: 15) | NO.: 16) | NO.: 12) | NO.: 17) | NO.: 18) | NO.: 19) |
| [C(RW)₄C] | [CR₅W₄C] | [R₅W₄] | W₂[CR₆C]W₂ | R₂[CW₆C]R₂ | [W₄-βAla-R₄-βAla] |
| (SEQ ID | (SEQ ID | (SEQ ID | (SEQ ID | (SEQ ID | (SEQ ID |
| NO.: 20) | NO.: 21) | NO.: 22 | NO.: 23) | NO.: 24) | NO.: 5) |
| [C(RW)₅C] | [CR₆W₄C] | [R₆W₄] | W₂[CR₇C]W₂ | R₂[CW₇C]R₂ | [W₃-βAla-R₃-βAla] |
| (SEQ ID | (SEQ ID | (SEQ ID | (SEQ ID | (SEQ ID | (SEQ ID |
| NO.: 25) | NO.: 26) | NO.: 27) | NO.: 28) | NO.: 29) | NO.: 30) |
| [C(RW)₆C] | [CR₇W₄C] | [R₇W₄] | W₃[CR₅C]W₃ | R₃[CW₅C]R₃ | [W₂-βAla-R₂-βAla] |
| (SEQ ID | (SEQ ID | (SEQ ID | (SEQ ID | (SEQ ID | (SEQ ID |
| NO.: 31) | NO.: 32) | NO.: 33) | NO.: 34) | NO.: 35) | NO.: 36) |
| [C(RW)₇C] | [CR₄W₅C] | [R₄W₅] | W₃[CR₆C]W₃ | R₃[CW₆C]R₃ | [W₄-βAla₂-R₄-βAla₂] |
| (SEQ ID | (SEQ ID | (SEQ ID | (SEQ ID | (SEQ ID | (SEQ ID |
| NO.: 37) | NO.: 38) | NO.: 39) | NO.: 40) | NO.: 41) | NO.: 42) |
| [C(RW)₈C] | [CR₄W₆C] | [R₄W₆] | W₃[CR₇C]W₃ | R₃[CW₇C]R₃ | [W₅-βAla-R₅-βAla] |
| (SEQ ID | (SEQ ID | (SEQ ID | (SEQ ID | (SEQ ID | (SEQ ID |
| NO.: 43) | NO.: 44) | NO.: 45) | NO.: 46) | NO.: 47) | NO.: 48) |
| [C(RW)₉C] | [CR₄W₇C] | [R₄W₇] | W₄[CR₅C]W₄ | R₄[CW₅C]R₄ | [W₄-βAla-R₄-βAla] |
| (SEQ ID | (SEQ ID | (SEQ ID | (SEQ ID | (SEQ ID | (SEQ ID |
| NO.: 49) | NO.: 50) | NO.: 51) | NO.: 52) | NO.: 53) | NO.: 54) |
| [C(RW)₁₀C] | [CR₅W₅C] | [R₅W₅] | W₄[CR₆C]W₄ | R₄[CW₆C]R₄ | [W₃-βAla-R₃-βAla] |
| (SEQ ID | (SEQ ID | (SEQ ID | (SEQ ID | (SEQ ID | (SEQ ID |
| NO.: 55) | NO.: 4) | NO.: 57) | NO.: 58) | NO.: 59) | NO.: 60) |
| [(RW)₅] | [CR₆W₅C] | [R₆W₅] | W₄[CR₇C]W₄ | R₄[CW₇C]R₄ | [W₂-βAla-R₂-βAla] |
| (SEQ ID | (SEQ ID | (SEQ ID | (SEQ ID | (SEQ ID | (SEQ ID |
| NO.: 61) | NO.: 62) | NO.: 63) | NO.: 65) | NO.: 65) | NO.: 66) |
| [K(RW)₅E] | [CR₇W₅C] | [R₇W₅] | W₅[CR₅C]W₅ | R₅[CW₅C]R₅ | [W₄-βAla₂-R₄-βAla₂] |
| (SEQ ID | (SEQ ID | (SEQ ID | (SEQ ID | (SEQ ID | (SEQ ID |
| NO.: 67) | NO.: 68) | NO.: 69) | NO.: 70) | NO.: 71) | NO.: 72) |
| Linear | [CR₅W₆C] | [R₅W₆] | W₅[CR₆C]W₅ | R₅[CW₅C]R₅ | |
| (C(RW)ₙC | (SEQ ID | (SEQ ID | (SEQ ID | (SEQ ID | |
| | NO.: 73) | NO.: 74) | NO.: 75) | NO.: 76) | |
| | [CR₅W₇C] | [R₅W₇] | W₅[CR₇C]W₅ | R₅[CW₇C]R₅ | |
| | (SEQ ID | (SEQ ID | (SEQ ID | (SEQ ID | |
| | NO.: 77) | NO.: 78) | NO.: 79) | NO.: 80) | |
| | [CR₆W₆C] | [R₆W₆] | (W)ₙ[KR₇K](W)ₙ | Linear | |
| | (SEQ ID | (SEQ ID | | RₙCWₙCRₙ | |
| | NO.: 81) | NO.: 82) | | | |
| | [CR₇W₇C] | [R₇W₇] | Linear | | |
| | (SEQ ID | (SEQ ID | WₙCRₙCWₙ | | |
| | NO.: 83) | NO.: 84) | | | |
| | [CR₈W₈C] | [R₈W₈] | Linear | | |
| | (SEQ ID | (SEQ ID | (W)ₙ(KRₙK)(W)ₙ | | |
| | NO.: 85) | NO.: 86) | | | |
| | [CR₉W₉C] | [R₉W₉] | (Linear | | |
| | (SEQ ID | (SEQ ID | W)ₙ(Rₙ)(W)ₙ | | |
| | NO.: 87) | NO.: 88) | | | |
| | [CR₁₀W₁₀C] | [R₁₀W₁₀] | | | |
| | (SEQ ID | (SEQ ID | | | |
| | NO.: 89) | NO.: 90) | | | |
| | [KR₄W₄E] | | | | |
| | (SEQ ID | | | | |
| | NO.: 91) | | | | |
| | [RₙCWₙC] | | | | |
| | 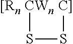 | | | | |
| | 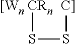 | | | | |

Linear, cyclic, hybrid cyclic-linear peptides were synthesized that have hydrophobic positively-charged, and/or cysteine residues as the amino acid sequence in its basic structure. Inventors varied the amino acid constituents in the structure to determine the effectiveness of derived compounds in siRNA binding and delivery and to establish a structure-activity relationship. The strategy was to vary net hydrophobicity and the positive charges of derived compounds based on structure-activity relationships and evaluate siRNA binding, delivery, and silencing potentials. The rationale of current studies was that cancer cells have a higher percentage of glutathione relative to normal cells. Accordingly, disulfide bonds can be reduced in cancer cells to a significantly higher degree than normal cells, providing selective delivery of siRNA to cancer cells.

Compounds disclosed herein represent a new class of transfection delivery agents. The structures of these series of compounds are different than those of current delivery agents. The used amino acids, peptide sequence, examples of their structures, in vitro siRNA binding affinity, cytotoxicity, siRNA delivery, and silencing potential are summarized herein.

Preferred compound(s) could be used as a stand-alone or to be used in generating peptide-capped gold or silver nanoparticles for siRNA delivery. These peptides can be used in combination with siRNA. in different ratios. Alternatively, peptide-capped nanoparticles of these peptides can be combined with siRNA. The peptides can be used alone or in combination with nanoparticles and peptide-capped nanoparticles. Examples of suitable nanoparticles include metal nanoparticles (e.g., gold and/or silver nanoparticles) that can be used in combination with peptides to improve the delivery of siRNA. Cell-penetrating peptide-capped nanoparticles with antimicrobial properties will be preferentially taken up by the cells where they gradually release their cargo siRNA, resulting in sustained local silencing effect by a two-pronged mechanism without causing significant toxicity to normal cells. Peptides-capped metal nanoparticles have cell-penetrating properties as membrane permeabilizers. Cell-penetrating peptides can entrap siRNA. and enhance the uptake of siRNA across the membrane when they cap the metal nanoparticles.

Compounds disclosed herein represent a new class of nucleic delivery agents. The structures of these series of compounds are different than those of current delivery agents. The used amino acids, peptide sequence, examples of their structures, in vitro siRNA binding affinity, cytotoxicity, siRNA delivery, and silencing potential are summarized herein.

Amino acids: Examples of suitable positively-charged amino acids in the linear and cyclic peptides are L-arginine, L-lysine, L-histidine, D-histidine, D-arginine, D-lysine. Furthermore, positively-charged amino acids are L- or D-arginine and L- or D-lysine, ornithine, L- or D-histidine residues with shorter or longer side chains (e.g., C3-Arginine (Agp), C4-Arginine (Agb)), diaminopropionic acid (Dap) and diaminobutyric acid (Dab), amino acids containing free side-chain amino or guanidine groups, and modified arginine and lysine residues.

Examples of suitable hydrophobic residues in the linear and cyclic peptides are L-tryptophan, D-tryptophan, L-phenylalanine, D-phenylalanine, L-isoleucine, D-isoleucine, p-phenyl-L-phenylalanine (Bip), 3,3-diphenyl-L-alanine (Dip), 3(2-naphthyl)-L-alanine (NaI), 6-amino-2-naphthoic acid, 3-amino-2-naphthoic acid, 1,2,3,4-tetrahydronorharmane-3-carboxylic acid, 1,2,3,4-tetrahydro-3-isoquinolinecarboxylic acid (Tic-OH), 1,2,3,4-tetrahydro-3-isoquinolinecarboxylic acid, modified d- or 1-tryptophan residues like N-alkyl or N-aryl tryptophan, substituted d- or L-tryptophan residues (e.g., 5-hydroxy-L-tryptophan, 5-methoxy-L-tryptophan, 6-chloro-L-tryptophan), other N-heteroaromatic and hydrophobic amino acids, and fatty amino acids (e.g., NH₂-(CH₂),COOH, where x=1-20).

Sequence: A preferred sequence of these peptides includes linear (C(XY)ₙC) or cyclic [(C(XY)ₙC)] (through disulfide bridge or N- to C-terminal or both), linear [C(X)ₙ(Y)ₙC)] or cyclic [(C(X)ₙ(Y)ₙ)C)] (through disulfide bridge or N- to C-terminal or both), linear (Y)ₙCXₙC(Y)ₙ, (X)ₙCYₙC(X)ₙ or hybrid cyclic-linear (Y)ₙ[CXₙC](Y)ₙ or hybrid cyclic-linear (X)ₙ[CYₙC](X)ₙ or hybrid cyclic-linear (Y)ₙ[KXₙK](Y)ₙ or hybrid cyclic-linear (X)ₙ[KYₙK](X)ₙ or linear (X)ₙ(KYₙK)(X)ₙor linear (X)ₙ(Yₙ)(X)ₙ, wherein X is a positively-charged amino acid, Y is a hydrophobic residue or [Xₙ-(β-alanaine)ₓ-Yₙ-(β-alanaine)ₓ], wherein n=1-10. Other amino acids can be inserted between positively-charged, between hydrophobic residues, or between positively-charged and hydrophobic residues while multiple positively-charged residues or multiple hydrophobic amino acids are next to each other, creating a positively-charged component on one side and a hydrophobic component on the other side. Similar or different positively charged or hydrophobic residues may be in the same peptide. In other words, positively charged amino acids can be the same or different. Similarly, hydrophobic amino acids in the same sequence can be the same or different. The peptides can have hybrid structures with cyclic peptides contain positively-charged residues or hydrophobic residues attached to two linear hydrophobic or positively-charged residues. Some of the sequences are shown in Table 1 and FIGS. 4-7.

In some examples, peptides (cyclic [CR₄W₄C] (SEQ NO.: 16) and hybrid W:₄[CR₅C]W₄) (SEQ ID NO.: 52) are disclosed; and other peptides in each group are inexpensive, easy-to-use, nontoxic, serum-stable, and efficient transfecting agent for all mammalian cells and in vitro nucleic acid delivery.

In one aspect, disclosed are CPs and hybrid cyclic-linear peptides (HCLPs). The concept of using a cyclic peptide-based transfection agent will provide intrinsic property associated with a conformationally constrained structure, such as selectivity to a class of siRNA or cell line, stability, and protection to siRNA from nuclease and serum, and low toxicity. CPs and HCLPs synthesized from natural L-amino acid will be biodegradable, leading to minimum toxicity as compared to lipid or polymer-based transfection agents. The CPs-based transfection agent will be economical as compared to lipid or polymer-based transfecting agents. Structural design of the CPs in the proposed study originated after extensive modification of previously developed classes of CPs containing W and R residues in the cyclic ring ([WR]ₙ; n=4,5) or R/W residues in the cyclic ring followed by a linear chain of R/W residues (called HCLPs: [R₅K]W₅ (SEQ ID NO.: 92) and [R₆K]W₆(SEQ ID NO.: 93)) or R residues in the cyclic ring containing chain of fatty acids ([(R₅(K-C16)₂], [R₅(K-C18)₂]) that demonstrate diverse applications in the non-covalent delivery of siRNA., By controlling and balancing inter-residue electrostatic and hydrophobic interactions with the siRNA. molecules, several classes of CPs and HCLPs were synthesized and evaluated with variable cellularization and efficiency in siRNA delivery. Efforts in structural modification provided several lead peptides (cyclic [CR₄W₄C] (SEQ ID NO.: 16) and hybrid W₄[CR₅C]W₄(SEQ ID NO.: 52)), which demonstrated high transfection efficiency without toxicity, serum stability as compared to commercial agent lipofectamine. Lead peptide [CR₄W₄C] (SEQ ID NO.: 16) is cyclized through intramolecular disulfide bridge between cysteine residues, which seems important in the release of siRNA from peptide-siRNA. complex in the cytoplasm after being reduced with glutathione. The reduction of the disulfide bridge provides conformational changes in the peptide-siRNA. complex, which modulates the release of siRNA. Furthermore, these peptides are internalized independently of endocytic pathways, which is a significant and distinct property as compared to other drug delivery platforms. The unique and intrinsic property of lead peptides to spontaneously translocate across bilayers is distinct from the behavior of well-known, highly cationic CPPs, such as TAT and Arg₉, which do not translocate across synthetic bilayers and instead enter cells primarily by endocytosis. Endosomal entrapment limits nucleic acid transfection efficiency significantly, as the nucleic acids are trapped in endocytic compartments and cannot reach their cytoplasmic or nuclear targets. The other significant advantage of the proposed structures is their high stability and low toxicity. Highly cationic CPPs preferentially interact with particular cell types, have limited serum half-life, show toxicity, and require 9-15 residues of R for efficient cargo delivery. Compared to linear CPPs, which are susceptible to hydrolysis by endogenous peptidases, cyclic peptides are enzymatically more stable and non-toxic. CPs and HCLPs can be used for different nucleic acids (including clustered regularly interspaced short palindromic repeats or CRISPR). Unlike many commercially available transfection agents that could not even be considered for in vivo use, the versatile structure of the proposed peptides offers the possibility of future modifications, e.g., incorporation into gold nanoparticles, conjugation with polyethylene glycol (PEG), and/or targeting moieties for stealth properties and active targeting, respectively, which create clinically relevant delivery systems.

The peptides are amphipathic in nature and conformationally constrained due to the intramolecular disulfide bridge between cysteine residues (FIG. 4). Classes 1 and 2 comprise of the block (multiple residues) of R and W residues between two cysteines followed by a disulfide bridge, e.g., [CR₄W₄C] (SEQ ID NO.: 16), [C(RW)₄C] (SEQ ID NO.: 20)) whereas Class 3 consists of two linear chains of hydrophobic residues on a cyclic peptide of R residues formed through a disulfide bridge, e.g., W₄[CR₅C]W₄ (SEQ ID NO.: 52) or two linear chains of positively charged residues on a cyclic peptide of W residues formed through a disulfide bridge, e.g., R₄[CW₅C]R₄ (SEQ ID NO.: 53). The R and W residues in Classes 1 and 2 could be alternate or all the R residues on one side and W residues on the opposite side, respectively. Class 2 also includes N to C cyclic peptides containing R and W residues with R residues on one side and W residues on the opposite side, e.g. [R4W₄] ((SEQ ID NO.: 12). The lead peptides were synthesized using Fmoc/tBu solid-phase chemistry and fully characterized. Complexes were formed using scrambled siRNA and both peptides at a wide range of N/P ratios. The complexes formed with both peptides had a size of approximately 200 nm or lower and showed slightly positive zeta-potential, which increased at higher N/P ratios.

The structural modifications in the lead peptides were used to optimize them as efficient vectors for cellular delivery of siRNA. through enhancing their interactions with negatively charged siRNA and providing them protection from the nucleases, and mediating the efficient cellular uptake.

The balance of amphipathic character, density, and position of positive charges and hydrophobic moieties in the conformationally constrained structures of peptides generated an efficient transfecting agent.

Classes 1 and 2 CPs contain disulfide and amide bridges and were developed to generate a conformationally constrained structure. Cyclic peptides are found to be more resistant to cellular metabolic enzymes, such as proteases and nucleases. Furthermore, the higher level of glutathione at the cellular level in various disease stages will facilitate the reduction of the disulfide bridge of peptide in the CPs containing disulfide bridge and thus changing the constrained conformation of the peptide to a flexible structure (a linear form of peptide), releasing the siRNA. Changes in the conformation of peptides in the peptide/siRNA complex will help in releasing the cargo inside the cytoplasm.

The data indicated that the lead cyclic [CR₄W₄C] (SEQ ID NO.: 16) and hybrid W₄₁[CR₅C]W₄ (SEQ ID NO.: 52) peptides containing disulfide bridge demonstrated potential applications as transfecting agents. Classes 1 and 2 peptides explore both the role of the disulfide bridge and constrained cyclic structure in developing optimal transfecting agents.

Class 3 represents HCLP analogs, composed of hydrophobic linear chains attached to a cyclic peptide containing positively charged R residues or o linear chains of positively charged residues on a cyclic peptide of W residues.

### EXAMPLES

The following examples are put forth so as to provide those of ordinary skill in the art with a complete disclosure and description of how the compounds, compositions, articles, devices, and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric.

The peptides can be used as pegylated peptides with or without targeting moieties such as RGD, folic acid, transferrin, antibody for nucleic acid delivery.

All amino acid building blocks and preloaded amino acid on the resin used in this study were purchased from AAPPTEC. Other reagents, chemicals, and solvents were procured from Sigma-Aldrich. The final compounds used in further studies were purified with reversed-phase High-performance liquid chromatography from Shimadzu (LC-20AP) using a binary gradient system of acetonitrile 0.1% TFA and water 0.1% TEA and a reversed-phase preparative column (X Bridge BEH130 Prep C18, 10 μm 18×250 μm Waters, Inc). The chemical structure of linear, cyclic, and hybrid cyclic-linear peptides, intermediates, and final products were characterized by high-resolution MALDI-TOF (GT-0264) from Broker Inc.

General method for the synthesis of linear peptides. The synthesis of linear peptides was performed by Fmoc/tBu solid-phase peptide synthesis method. All the peptides were synthesized using Fmoc solid-phase peptide synthesis using appropriate resin and Fmoc-protected amino acids.

Protected amino acid-2-Cl trityl resin and Fmoc protected amino acids were used as the building blocks for the synthesis peptides on a scale of 0.3 mmol. 2-H-benzotriazol -1-yl)-1,1,3,3-tetramethyluronium hexafluorophosphate (HBTU)/N,N-diisopropylethylamine (DIPEA) were used as coupling and activating reagents, respectively. Piperidine in N,N-dimethylformamide (DMF) (20%, v/v) was used for Fmoc deprotection. The resultant peptides were cleaved from the resin, and all protecting groups were removed using a cleavage cocktail of TFA/anisole/EDT/thioanisole (90:5:3:2, v/v/v/v) for 3 h. Crude products were precipitated by the addition of cold diethyl ether purified by reverse phase HPLC using a gradient of 0-90% acetonitrile (0.1% TFA) and water (0.1% TFA) over 60 min with C-18 column. Purified peptides were lyophilized to yield a white powder. The chemical structures of all synthesized peptides were elucidated using matrix-assisted laser desorption/ionization (MALDI) time-of-flight (TOF) mass spectroscopy with α-cyano hydroxycinnamic acid (CHCA) as a matrix.

MALDI-TOF (m/z) for C(RW)₄C (SEQ ID NO.: 20) [C₇₄H₁₀₀N₂₆O₁₁S₂]: calcd,1592.7506; found, 1592.7797 []⁻; MALDI-TOF (m/z) for C(RW)₅C (SEQ ID NO.: 94) [C₉₁H₁₂₂N₃₂O₁₃S₂]: calcd,1934.9311; found, 1934.2925 [M]⁺; MALDI-TOF (m/z) for CR₄C(SEQ ID NO.: 16) [C₇₄H₁₀₀N₂₆O₁₁S₂]: calcd, 1592.7506; found, 1593.3369 [M+H]⁺; MALDI-TOF (m/z) for CR₅W₅C (SEQ ID NO.: 56) [C₉₁H₁₂₂N₃₂O₁₃S₂]: calcd,1934,9311; found, 1936.445 [M+2H]⁺; MALDI-TOF (m/z) for R₄CW₄C (SEQ ID NO.: 95) [C₇₄H₁₀₀N₂₆O₁₁S₂]: calcd, 1592.7506; found, 1593.957 [M+H]⁺; MALDI-TOF (m/z) for W₄CR₄C (SEQ ID NO.: 96) [C₇₄H₁₀₀N₂₆O₁₁S₂]: calcd,1592.7506; found, 1593,160 [M+H]⁺; MALDI-TOF (m/z) for W₂CR₅CW₂ (SEQ ID NO.: 97) [C₈₀H₁₁₂N₃₀O₁₂S₂]: calcd, 1748.8517; found, 1749.475 [M+H]⁺; MALDI-TOF (m/z) for W₃CR₅CW₃ (SEQ ID NO.: 98) [C₁₀₂H₁₃₂N₃₄O₁₄S₂]: calcd, 2121.0104; found, 2122.159 [M+H]⁺; MALDI-TOF (m/z) for W₄CR₅CW₄(SEQ ID NO.: 99) [C₁₂₄H₁₅₃N₃₉O₁₅D₂]: calcd, 2493.1850; found, 2494.2889 [M+H]⁺.

Synthesis of cyclic peptides through N- to C-cyclization. Amino acid loaded on trityl resin and Fmoc-amino acid building blocks were used for the synthesis on a scale of 0.3 mmol. HBTU/DIPEA was used as coupling and activating reagents, respectively. Piperidine DMF (20% v/v) was used for Fmoc deprotection. The side-chain protected peptides were detached from the resin by TFE/acetic acid/DCM [2:1:7 (v/v/v)] then subjected to cyclization using 1-hydroxy-7-azabenzotriazole (HOAT) and N,N′-diisopropylcarbodiimide (DIC) in an anhydrous DMF/DCM mixture overnight. All protecting group were removed with cleavage cocktail of TFA/anisole/thioanisole (90:2:5 v/v/v) for 3 h. The crude products were precipitated by the addition of cold diethyl ether purified using reverse-phase HPLC using a gradient of 0-90% acetonitrile (0.1% TFA) and water (0.1% TFA) over 60 min with C-18 column. The purified peptide was lyophilized to yield a white powder (100 mg). The chemical structure of all synthesized peptides was elucidated using matrix-assisted laser desorption/ionization (MALDI) time-of-flight (TOF) mass spectrometer.

MALDI-TOF (m/z) for [R₄CW₄C] (SEQ ID NO.: 95) [C₇₄H₉₈N₂₆O₁₀S₂]: calcd, 1574.7401; found, 1575.578 [M+H⁺].

Synthesis of cyclic peptides cyclized through a disulfide bridge. About 30 mg of the linear peptide was dissolved in a 10% DMSO-H₂O solution (150 ml). The reaction mixture was stirred for 24-72 h at room temperature in an open round-bottomed flask. The reaction mixture was injected directly in reverse phase HPLC using a gradient of 0-90% acetonitrile (0.1% TFA) and water (0.1% TFA) over 60 min with C-18 column. The purified peptide was lyophilized to yield a white powder (20 mg). The chemical structure of all synthesized peptides was elucidated using matrix-assisted laser desorption/ionization (MALDI) time-of-flight (TOF) mass spectrometer.

MALDI-TOF (m/z) for ([C(WR)₄C]

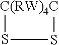

(SEQ ID NO.: 100) [C₇₄H₉₈N₂₆O₁₁S₂]: calcd, 1590.7350; found, 1590,8999 [M]^{+.}

MALDI-TOF (m/z) for [C(WR)₅C]

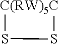

(SEQ ID NO.: 101) [C₉₁H₁₂₀N₃₂O₁₃S₂]: calcd,1932.9154; found, 1932.9976 [M]^{+.}

MALDI-TOF (m/z) for

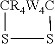

[C₇₄H₉₈N₂₆O₁₁S₂]: calcd, 1590,7350; found. 1591.161 [M+H]^{+.}

MALDI-TOF (m/z) for

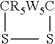

[C₉₁H₁₂₀N₃₂O₁₃S₂]: calcd, 1932.9154; found, 1937.6542 [M]^{+.}

MALDI-TOF (m/z) for

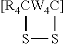

[C₇₄H₉₆N₂₆O₁₀S₂]: calcd, 1572.7244; found, 1573,148 [M+H]^{+.}

MALDI-TOF (m/z) for R₄[CW₄C] [C₇₄H₉₈N₂₆O₁₁S₂]: calcd, 1590,7350; found, 1590.873 [M]^{+.}

MALDI-TOF (m/z) for W₄[CR₄C][C₇₄H₉₈N₂₆O₁₁S₂]: calcd, 1590.7350; found, 1591.286 [M+H]^{+.}

MALDI-TOF (m/z) for

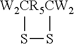

[C₈₀H₁₁₀N₃₀O₁₂S₂]: calcd, 1746.8410; found, 1746.994 [M]^{+.}

MALDI-TOF (m/z) for

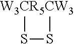

[C₁₀₂H₁₃₀N₃₄O₁₄S₂]: calcd, 2118.9991; found, 2119.1252 [M]^{+.}

MALDI-TOF (m/z) for

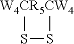

[C₁₂₄H₁₅₁N₃₉O₁₅S₂]: calcd, 2491.1850, found, 2491.5856 [M]^{+.}

MALDI-TOF (m/z) for (W)₂[KR₅K](W)₂[C₈₆H₁₂₄N₃₂O₁₁]: calcd, 1781.0127; found, 1781.61 [M]^{+.}

MALDI-TOF (m/z) for (W)[KR₅K](W) [C₆₃H₁₀₄N₂₈O₉]: calcd, 1408.8541; found, 1410,0432 [M+H]^{+.}

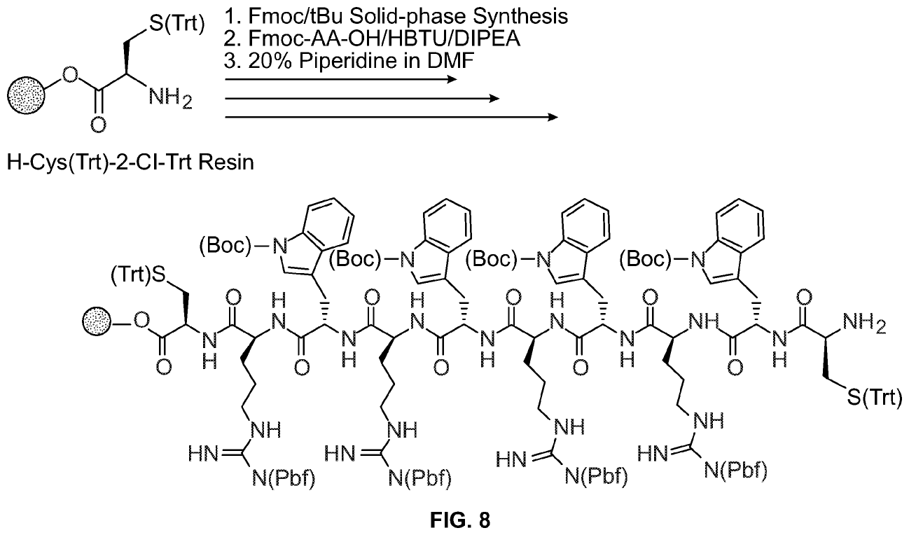

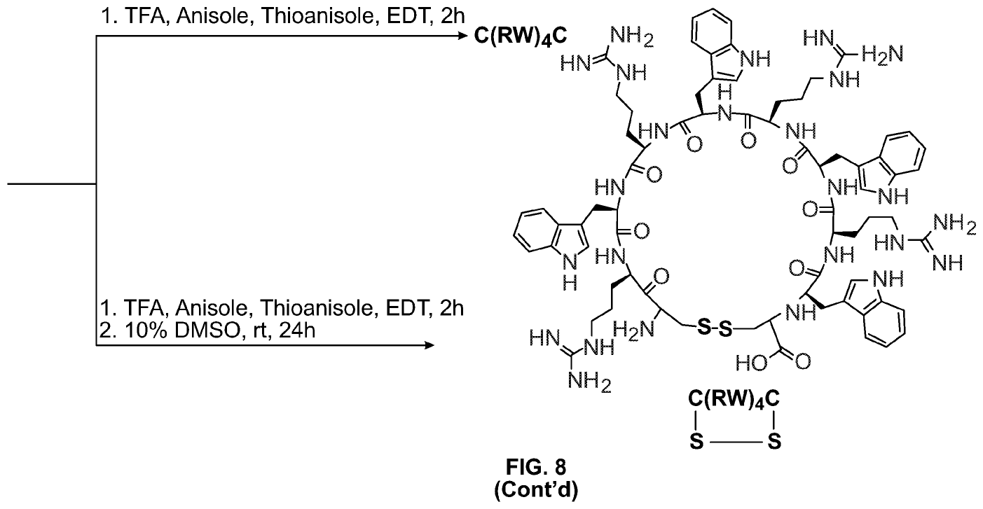

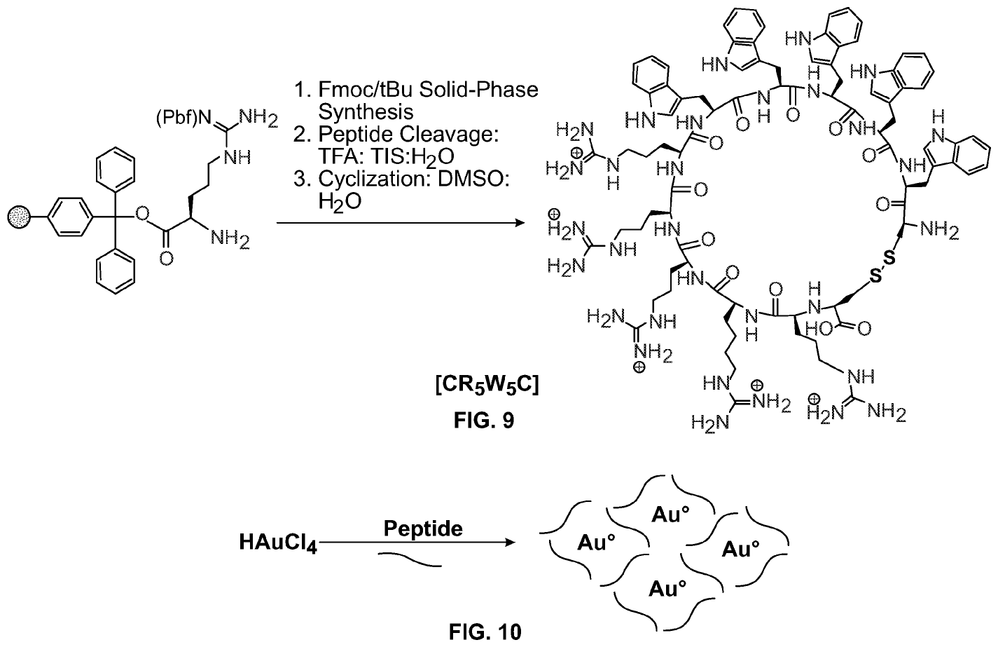

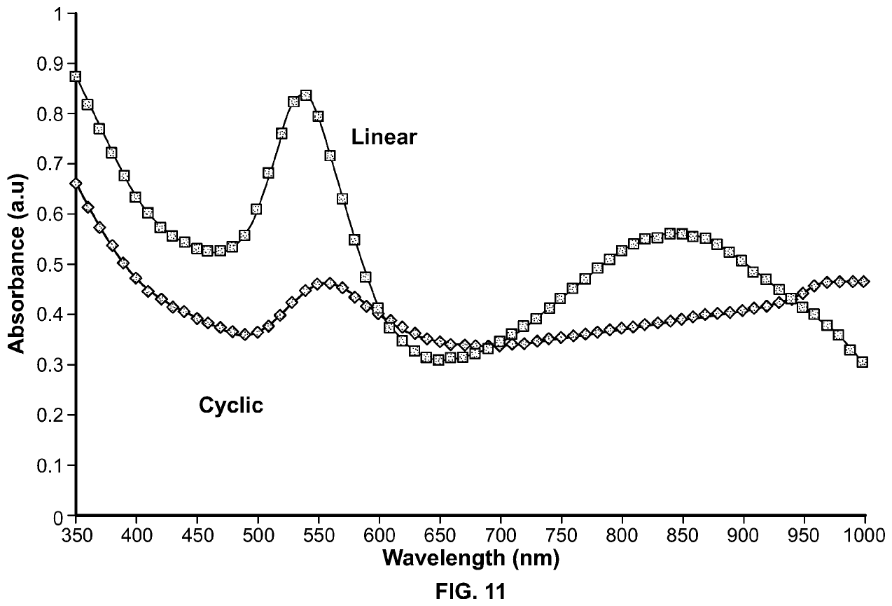

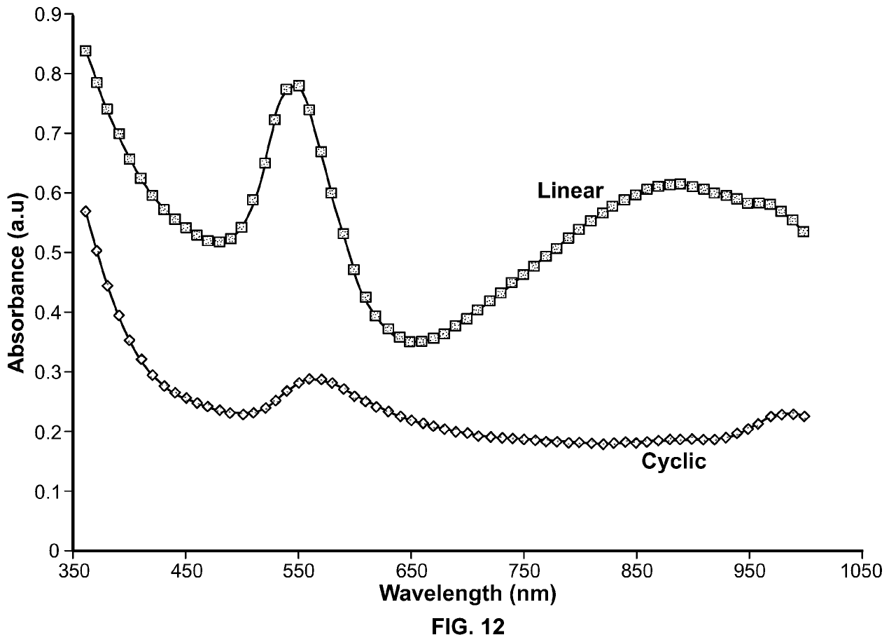

As representative examples, the synthesis of representative linear and cyclic peptides is shown in FIGS. 6 and 7, as explained previously (Mohammed et al., Comparative molecular transporter properties acyclic peptides containing tryptophan and arginine residues formed through disulfide cyclization. Molecules 2020, 25(11); E2581. doi: 10.3390/molecules 25112581). Parentheses ( )and brackets represent [ ] linear and cyclic peptides, respectively. Also, letters land c to represent linear and cyclic peptides. respectively. The linear peptide containing alternative tryptophan and arginine amino acids with two cysteine residues (CWRWRWRWRC) was assembled on the cysteine preloaded chlorotrityl resin using coupling and deprotecting reagents as depicted in FIG. 8. The linear peptide was completely cleaved in the presence of freshly prepared cleavage cocktail containing trifluoroacetic acid (TFA)/thioanisole/1,2-ethanedithiol (EDT)/anisole (90:5:3:2 v/v/v/v) to afford I(C(WR)₄C). The linear peptide was subjected to the oxidation reaction using 10% DMSO-H₂O solution over 24 h at room temperature to afford disulfide-linked cyclic peptide. Peptides were characterized and purified using matrix-assisted laser desorption/ionization (MALDI) mass spectroscopy and reverse-phase high-performance liquid chromatography (RP-HPLC), respectively. The analytical HPLC data showed a retention time (RT) of 9.6 and 9.8 min for the linear peptides (C(WR)4C) (SEQ ID NO.: 102) and (C(WR)5C) ((SEQ ID NO,:103), respectively, while the RT for cyclic peptides [C(WR)₄C](SEQ ID NO.: 100) and [C(WR)₅C](SEQ ID NO.: 101) were 10.3 and 10.5 min, respectively.

Synthesis of Gold nanoparticles. In a typical synthesis, an aqueous solution of peptide and chloroauric acid were mixed in the equimolar ratio (1:1) (2 mM peptide solution and 2 mM chloroauric acid) and stirred at room temperature for 24 hours (FIG. 10).

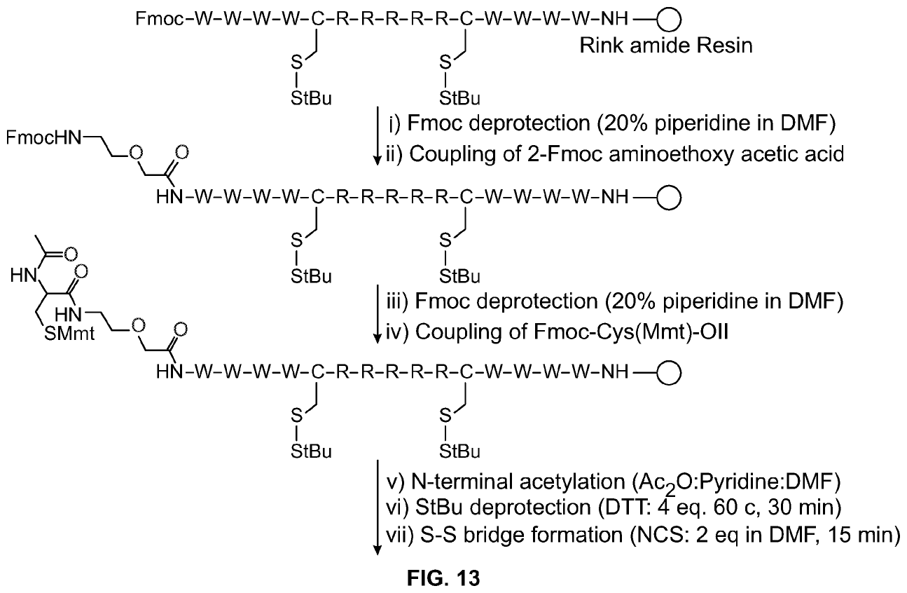

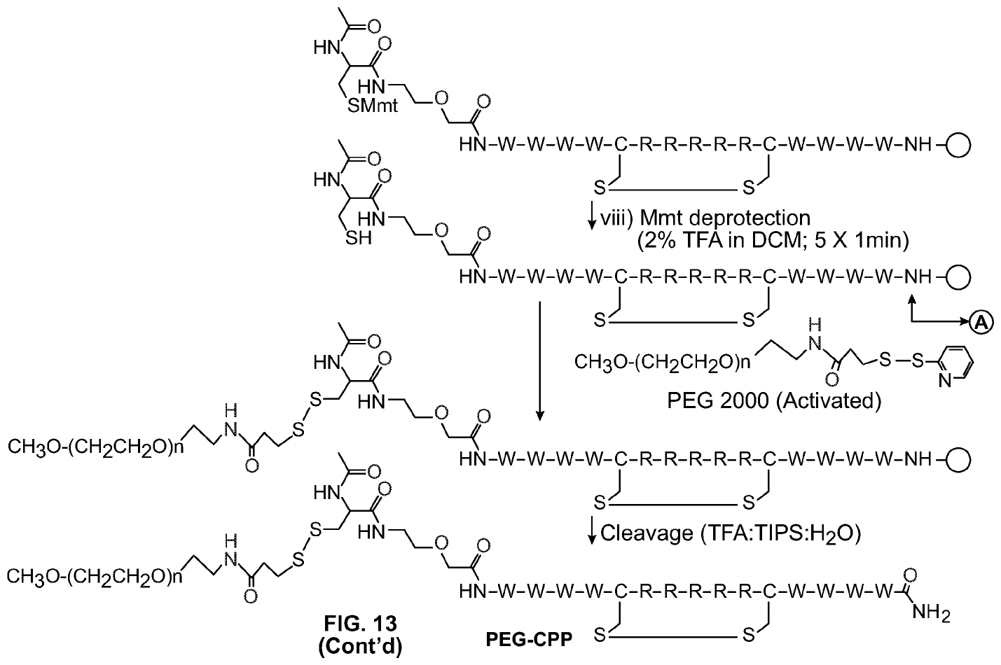

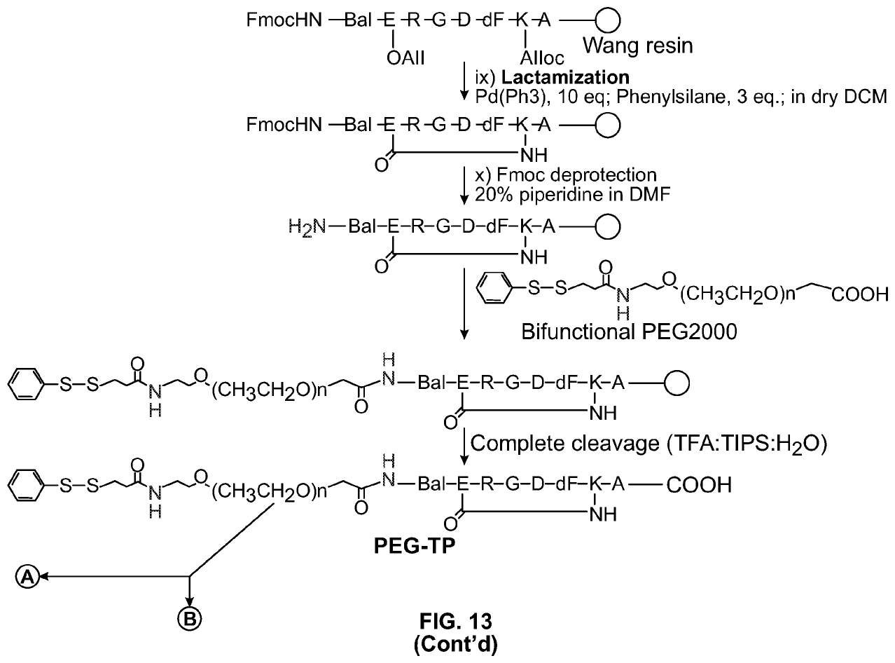

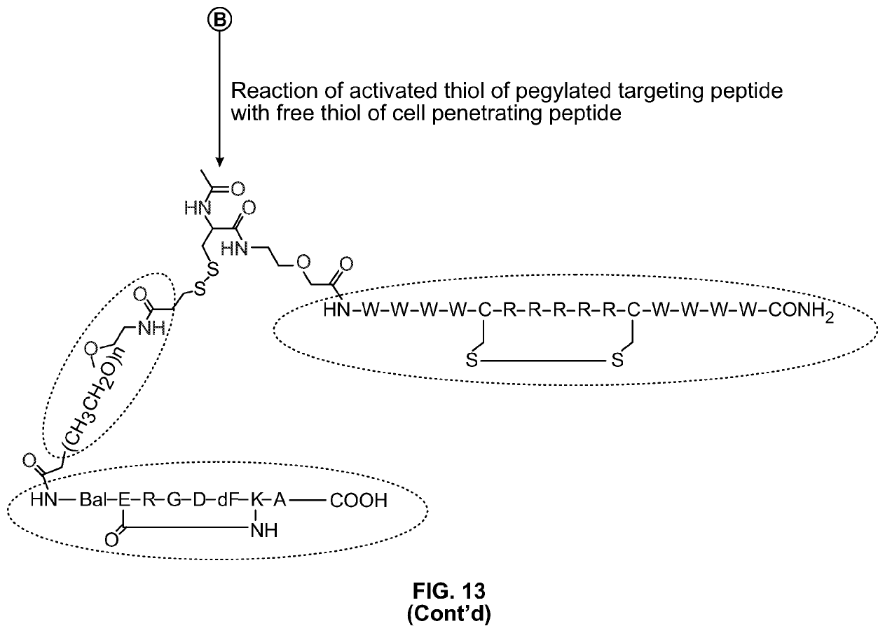

Synthesis of the pegylated cell-penetrating peptide (PEG-CPP). The solid-phase synthesis of the linear peptide sequence was conducted using the standard protocol mentioned before. The desired peptide sequence was assembled on rink amide resin. Orthogonal protected Cys(StBu) residues were incorporated to achieve on-resin sulfur bridge formation. N-Terminal of parent peptide was further extended by coupling 2-Fmoc aminoethoxy acetic acid as a spacer between the parent peptide and another cysteine residue Cys(Mmt), which was installed to pegylate the peptide by exploiting free side chain thiol functionality. The tent-butyl thiol groups were deprotected on resin using 3.8 mmol dithiothreitol (DTT) in 2.5 mL DMF with 0.25 mL DIEA. The reaction proceeded at 60° C. for 20 minutes. The resin was washed thoroughly with DMF and DCM. On-resin disulfide formation reaction was conducted using N-chlorosuccinimide (NCS, 2 eq.) in DMF Methoxytrityl group was deprotected selectively by repeatedly treating the peptidyl resin with 2% TFA in DCM. The resulted free thiol functionality was used for pegylation of the peptide by reacting with preactivated PEG (mPEG-OPSS; Orthopyridyl disulfide). For on-resin pegylation reaction, 2 eq. of preactivated PEG-SH was added to the peptidyl resin in DMF. The reaction mixture was kept for shaking at room temperature for around 14 hours (FIG. 13). The pegylated peptide was cleaved from the resin and purified by RP-HPLC and mass was confirmed by Q-TOF-LC-MS.

Synthesis of pegylated targeting peptide (PEG-TP). Synthesis of pegylated targeting peptide was conducted on wang resin as represented in the scheme (FIG. 13). Following the synthesis of the linear octameric peptide, on-resin lactamization was conducted through an amide bond formation between free side-chain carboxylic and amine functionality of Glu and Lys, respectively. The lactamization reaction was performed using tetrakis(tripbenylphosphine)palladium (10 eq.) and phenylsilane (3 eq.) in dry DCM. The reaction was proceeded by bubbling nitrogen and repeated twice for 15 min each. After completion of lactamization reaction, peptidyl resin was washed thoroughly with DMF and DCM. The targeting peptide was pegylated by amide bond formation between N-terminal free amine of peptide and free carboxylic group of bifunctional PEG (OPSS-PEG-COOH; Orthopyridyl disulfide PEG Carboxylic acid). The pegylated peptide was cleaved from resin and purified by RP-HPLC and mass was confirmed by Q-TOF-LC-MS.

Synthesis of the final conjugate (TP-PEG-CPP). As depicted in the scheme, the final conjugate was synthesized by sulfur bridge formation between pegylated targeting peptide (PEG-TP) and cell-penetrating peptide (CPP). Purified pegylated targeting peptide (2 equiv.) having preactivated thiol was added to peptidyl resin in DMF. The reaction mixture was kept for shaking at room temperature for around 14 hours. Final conjugate was cleaved from the resin and purified by RP-HPLC and mass was confirmed by Q-TOF-LC-MS.

Complex formation with modified peptides and siRNA. The peptide/siRNA complexes were formed in different N/P ratios for the variety of the in vitro studies conducted according to the previously reported procedure (Mozaffari et al., 2019). The complexes formed spontaneously based on ion/ion interaction in normal saline. For each formulation, the required amount of scrambled siRNA stock solution (10 μM) was added to normal saline, and then, the calculated amount of peptide stock solution (1 mg/mL) was added to the solution. The mixture was gently mixed and was incubated at room temperature for 30 minutes to ensure complete complexation. The peptide:siRNA ratios used in complex formations are reported as weight/weight ratios, as well as nitrogen/phosphate (N/P) ratios. The N/P ratios were calculated using the following formula:

NP=(#of Moles peptide×# of nitrogens in each molecule of peptide) (#of Moles of siRNA×# of phosphate groups in each molecule)

All siRNAs used in this project contain 21-25 base pairs in each strand. The scrambled siRNA used in this set of experiments contains 23 base pairs.

Size and Surface Charges. The efficiency of siRNA carriers largely depends on the size and overall surface charge of the complex. Dynamic light scattering (DLS) was used to determine the size and ζ-potential at different N/P ratios to determine the optimal N/P ratio for a compact complex (50-200 nm) and slightly positive charge.

siRNA binding affinity. To evaluate binding affinity to siRNA, a study was designed based on the quantification of free siRNA using SYBR Green II. Peptides were mixed with siRNA with a wide range of N/P ratios (0.05-40), and the amount of unbound siRNA was quantified by the fluorescent signal of SYBR Green II. The required N/P ratio for 50% binding (BC50) was calculated for each peptide.

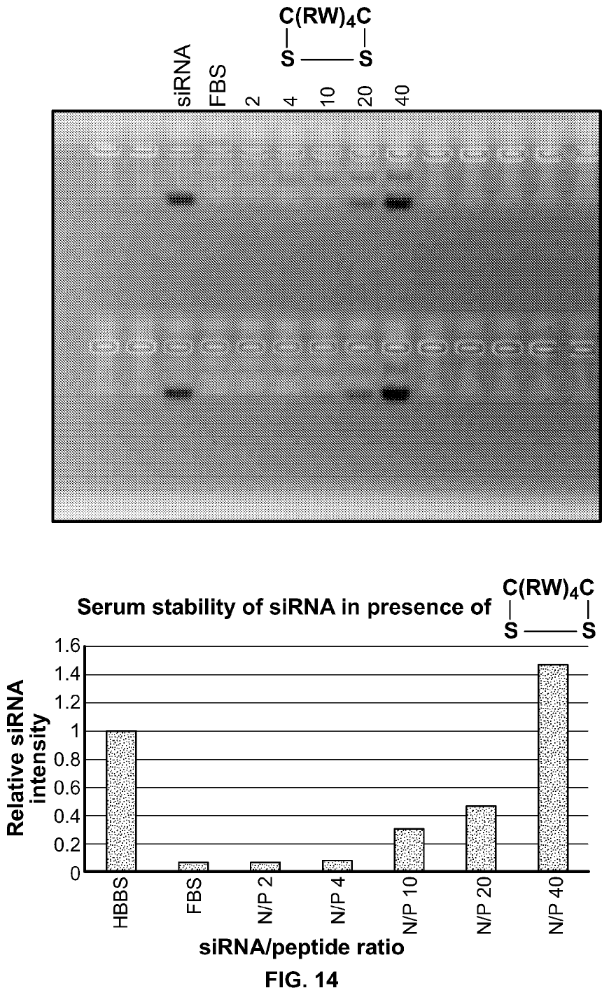

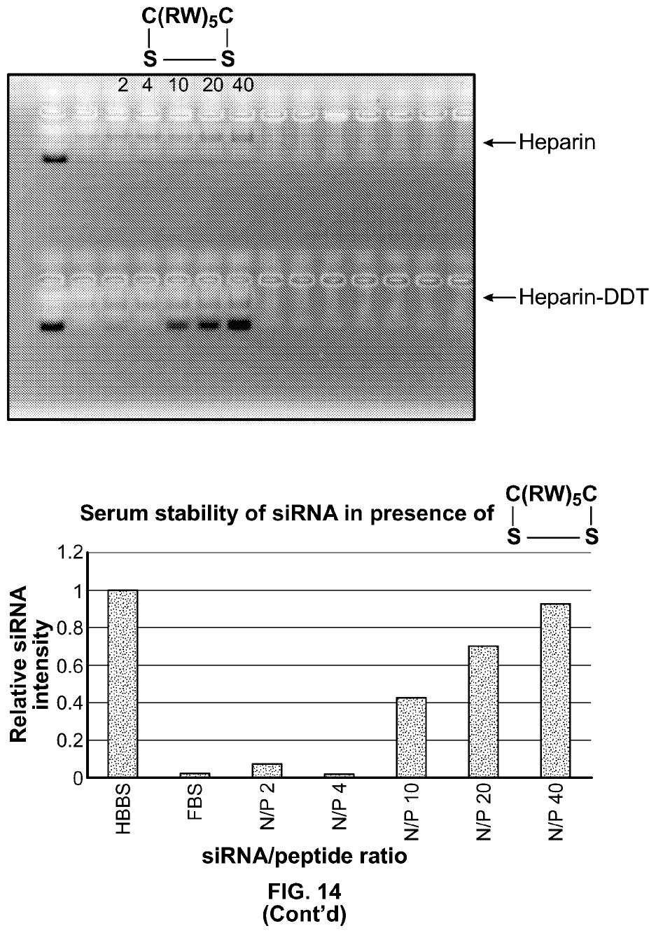

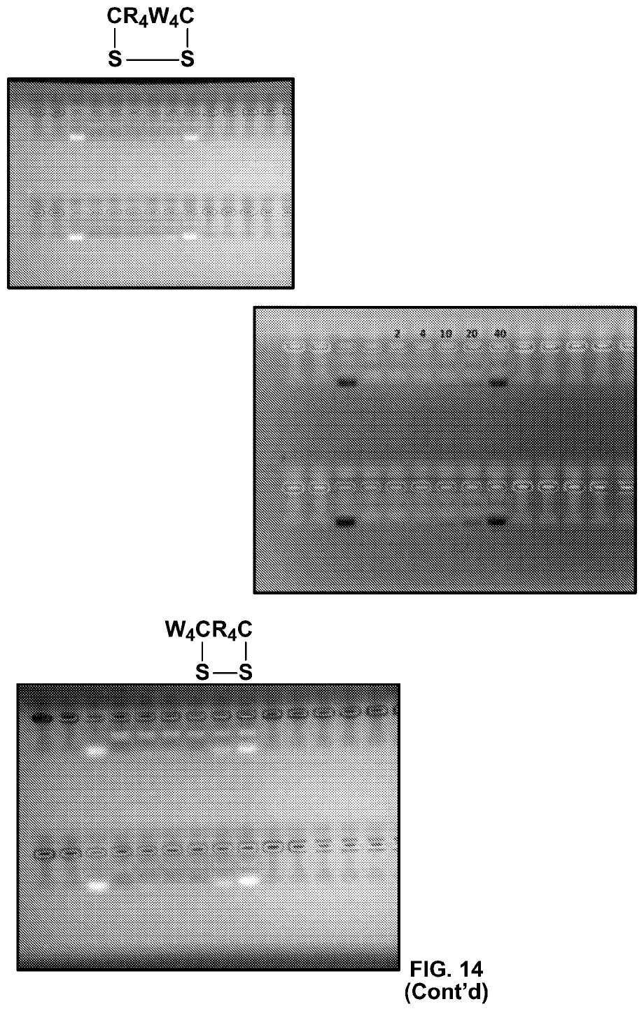

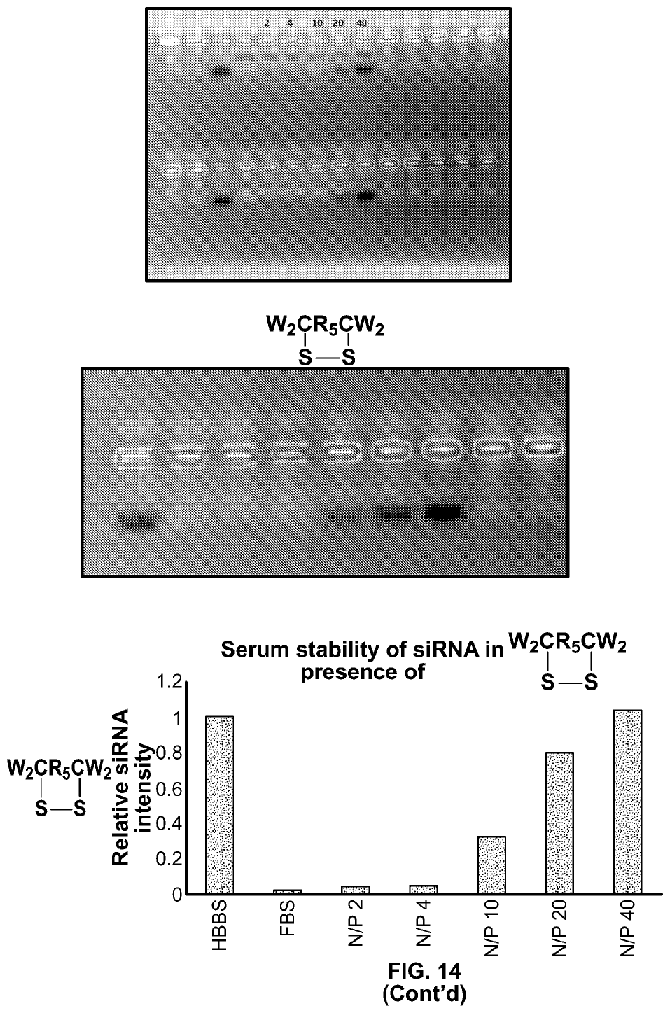

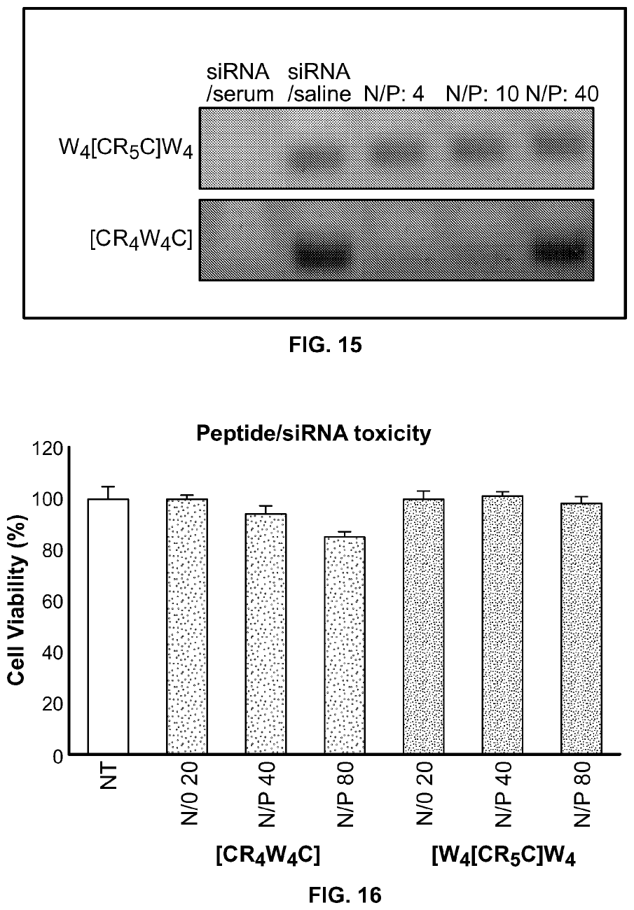

Serum Stability. In order to determine the capability of the complexes to protect the siRNA against enzymatic degradation, Inventors exposed different study groups to a diluted fetal bovine serum (FBS) solution, with “naked” siRNA as a positive control, and siRNA exposed to saline as a negative control according to the previously reported procedure (Mozaffari et al., 2019), and the results are summarized in FIG. 14. A higher concentration of FBS (25%) was used to determine the stability of siRNA in a harsher condition and a more biologically-relevant environment. After 24 hours of exposure, siRNA bands were quantified by electrophoresis. After 24 h exposure to serum, Inventors did not observe any bands for siRNA in the positive control groups, which indicated complete degradation of “naked” siRNA in these conditions, and validated the method. All peptides were able to protect siRNA. almost completely after 24 h incubation with the serum at 37° C. at N/P ratios of 40. Significant protection that approached 100% with increasing the N/P ratio to 40:1.

The binding affinity for [CR₄W₄C](SEQ ID NO.: 16) and W₄[CR₅C]W₄(SEQ ID NO.: 52) showed that approximately 4 and 10 N/P ratios are required for 50% binding. While “naked” siRNA. was completely degraded in the presence of serum, both peptides were able to protect siRNA completely after 24 hours of exposure to the serum (as compared to siRNA exposed to saline as negative control) at an N/P ratio of 40 (FIG. 14). The toxicity of the peptides was evaluated in triple-negative breast cancer (TNBC) cells MDA-MB-231 (as a model cancer cell line) with peptide alone and siRNA complexes, and CCK assay to quantify cell viability after 48 hours.

Protection of siRNA against Enzymatic Degradation. In order to study the capability of the fatty acid-conjugated linear and cyclic peptides to enhance the stability of siRNA in biological environments, samples of unprotected scrambled siRNA (positive control) and peptide/siRNA complexes at a different peptide:siRNA ratios were exposed to fetal bovine serum (FBS) solutions. After completion of complex formation for each formulation, each sample was added to a 25% v/v FBS solution in HBSS, and the mixture was incubated at 37° C. for 24 h. A sample of unprotected siRNA in HBSS was used as a negative control, representing 100% intact siRNA. After the incubation, complexes were dissociated using a 2:3 mixture of heparin (5% solution in normal saline) and EDTA (0.5 mM), and the samples were analyzed using a 1% agarose gel (with 1 μg/mL ethidium bromide) at 70 V for 20 minutes. UV illumination (Gel-Doc system, Bio-Rad; Hercules, CA) was used to visualize the gel, and the intensity of the bands (representing remaining intact siRNA) was quantified by Image J software.

Cylotoxicity Assays. The cytotoxicity of the peptides was evaluated in a panel of cancer and non-cancerous cell lines. Normal human breast (Hs 578Bst, ATCC™ HTR-125™), kidney (LLCP-K1, ATCC CRL-1392), prostate (RWPE-1; ATCC™ CRL-11609™), colon (CCD-33Co; ATCC™ CRL-1539™), heart (H9C2. ATCC CRL 1446) or endometrial (KC02-44D hTERT, ATCC™ SC-6000™) cell lines were exposed to a wide range of peptide/siRNA concentrations. Due to the popularity of siRNA silencing in cancer research, the toxicity of the same study groups were also evaluated in TNBC cells MDA-MB-231 (ATCC™ HTB-26™), MDA-MB-468 (ATCC™ HTB-132), ovarian SK-OV-3 (ATCC™ HTB-77™), colorectal ht-29 (ATCC™ HTB-38™), and prostate LNCaP (ATCC™ CRL-174™) cancer cell lines. Cell viability was evaluated by CCK assay after 48 hours of exposure. Neither of the peptides showed significant toxicity in the range of concentration or N/P ratio evaluated (FIG. 16).

The human breast cancer cell line MDA-MB-231 was purchased from American Type Culture Collection (ATCC; Manassas, Va.). MDA-MB-231 was cultured in the DMEM medium. The media was supplemented with 10% (v/v) fetal bovine serum, 100 U/mL penicillin, and 100 μg/ml streptomycin. Cells were maintained in the normal condition of 37° C. and 5% CO₂ under a humidified atmosphere and were sub-cultured when 80-100% confluent.

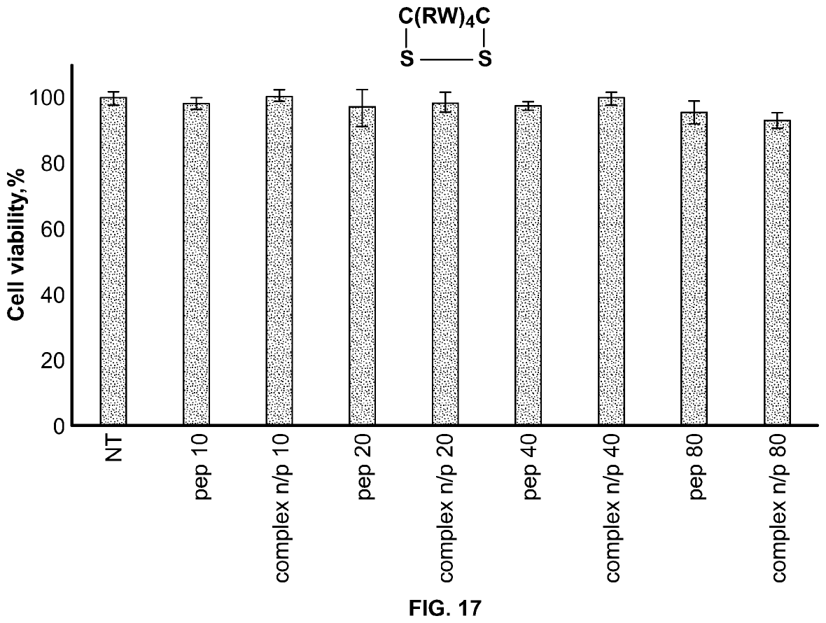

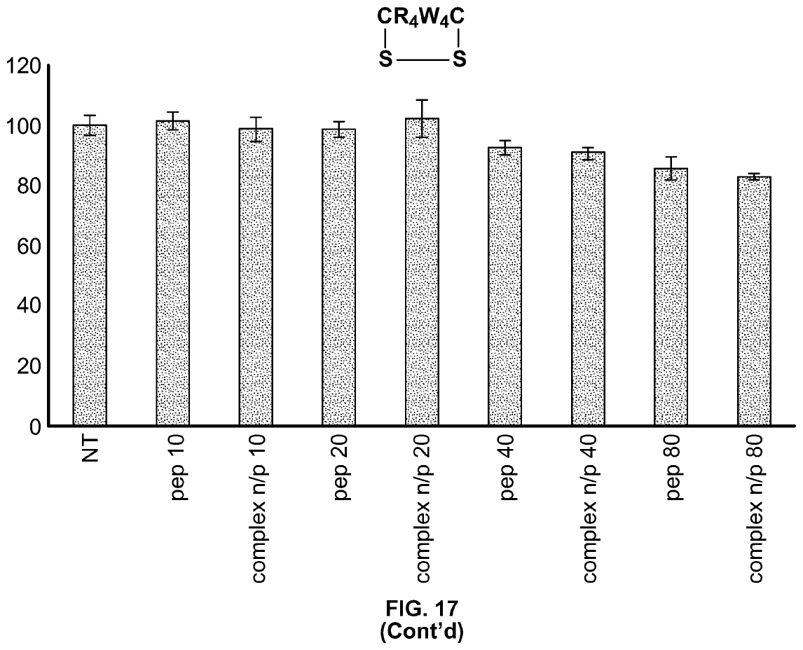

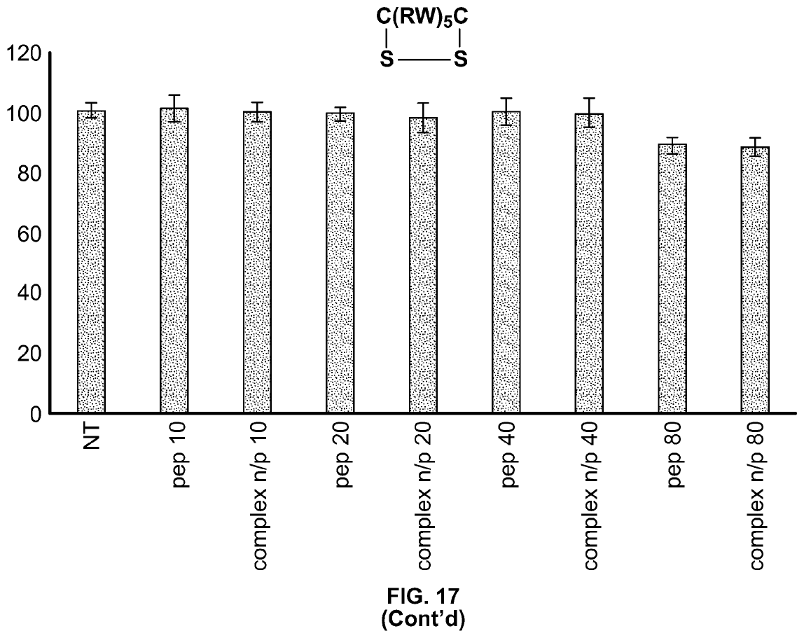

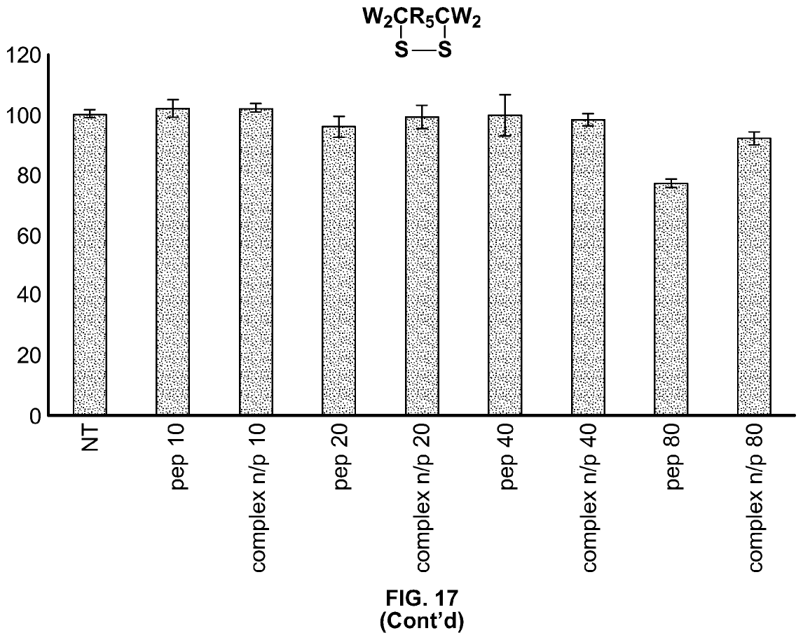

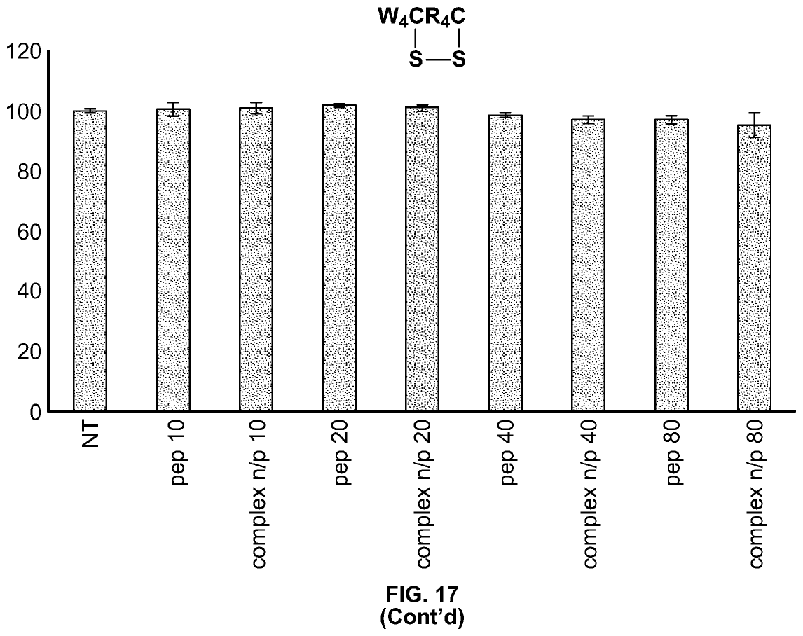

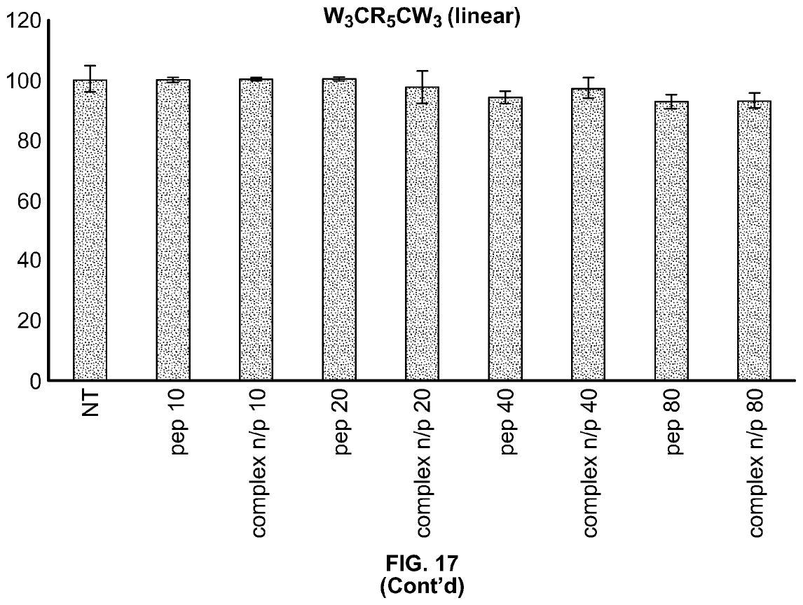

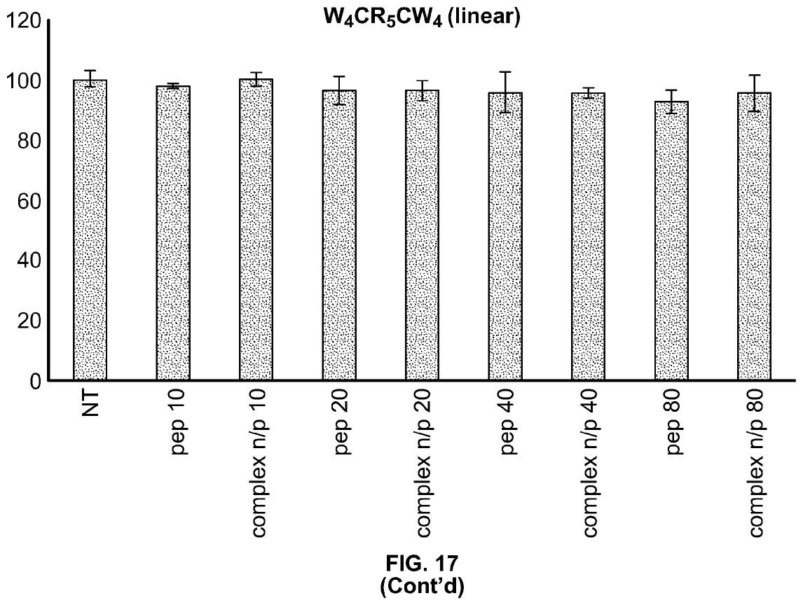

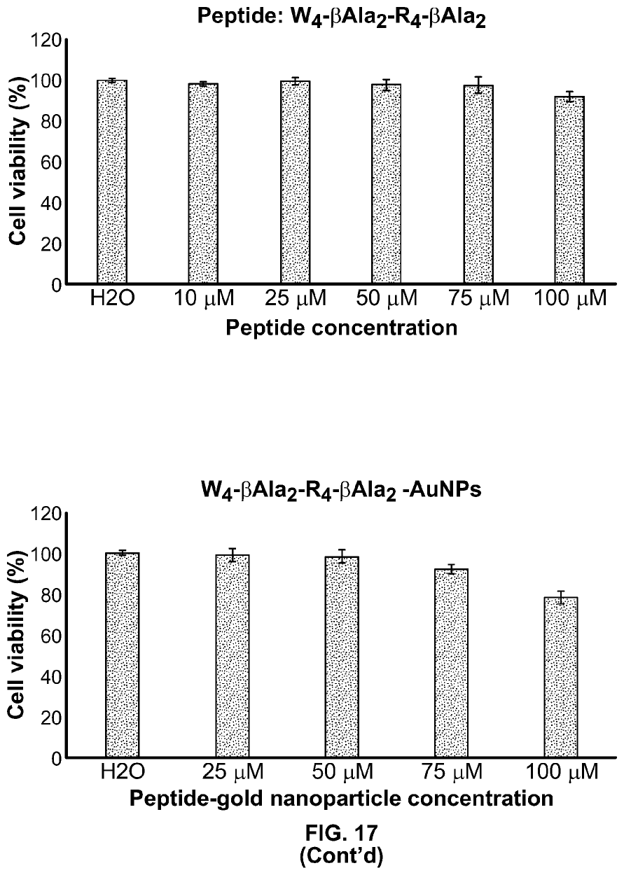

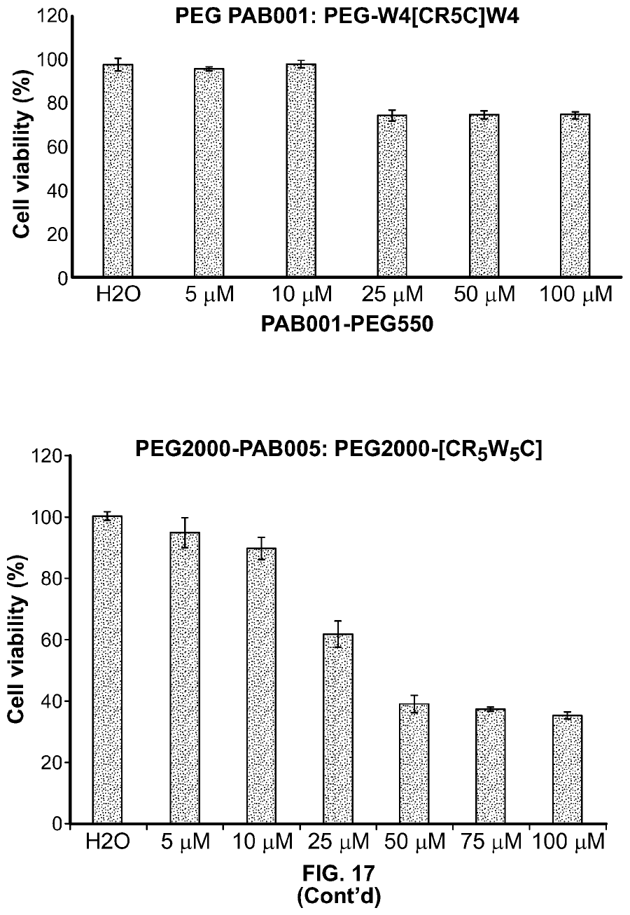

The &Tea of peptides on the viability of human cell lines was evaluated by exposing the MDA-MB-231 cell lines (FIG. 17) to different concentrations of peptide solution and exposing the cells to peptide/siRNA complexes prepared with different peptides at a various peptide:siRNA ratios according to the previously reported procedure by us (Mozaffari et al., 2019). Confluent cultures (≈800,000 cells/mL) of MDA-MB-231 cancer cell lines were seeded in 96-well plates. After 24 h, cells were exposed to different concentrations of peptide solutions or peptide/siRNA complexes. The final concentration of scrambled siRNA was 36 nM in all cytotoxicity studies performed with peptide/siRNA complexes. The cells were then incubated for 48 hours at 37° C. with controlled CO₂ and humidity. A standard 3-(4,5-dimethylthiazol-2-yl)-2,5-diphenyttetrazolium bromide (MTT) assay was performed after the incubation period to determine the cell viability, as a. percentage of untreated (NT) cells. In MDA231 cells, the peptides did not show significant cytotoxicity at a N/P ratio 40.1, both alone and incorporated in the siRNA complex.

Cellular Uptake. Cellular uptake was quantified using flow cytometry and was evaluated in all the cell lines included in the cytotoxicity study. However, since this method did not confirm cytoplasmic accumulation, confocal microscopy was performed to confirm and visualize the cytoplasmic delivery. Free siRNA and Lipofectamine™ were used as negative and positive controls, respectively.

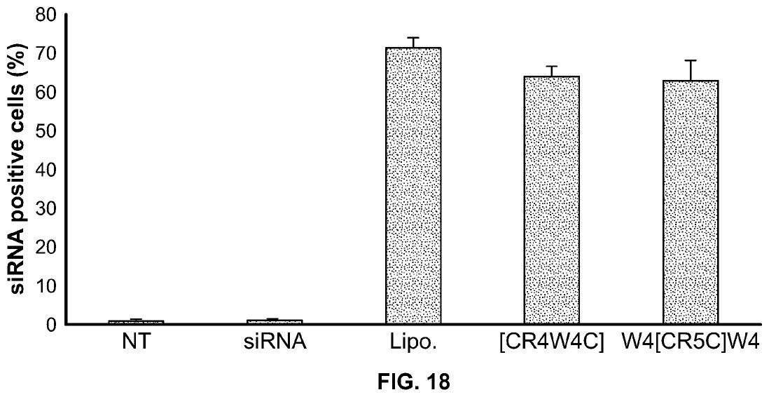

Cellular internalization was studied in MDA-MB-231 using flow cytometry and confocal microscopy. The percentage of cells identified as siRNA-positive for the complexes formed with two lead peptides using an N/P ratio of 20 was comparable to the percentage of cells achieved using commercially available Lipofectamine™ 2000 (˜63% vs. 72%; FIG. 18).

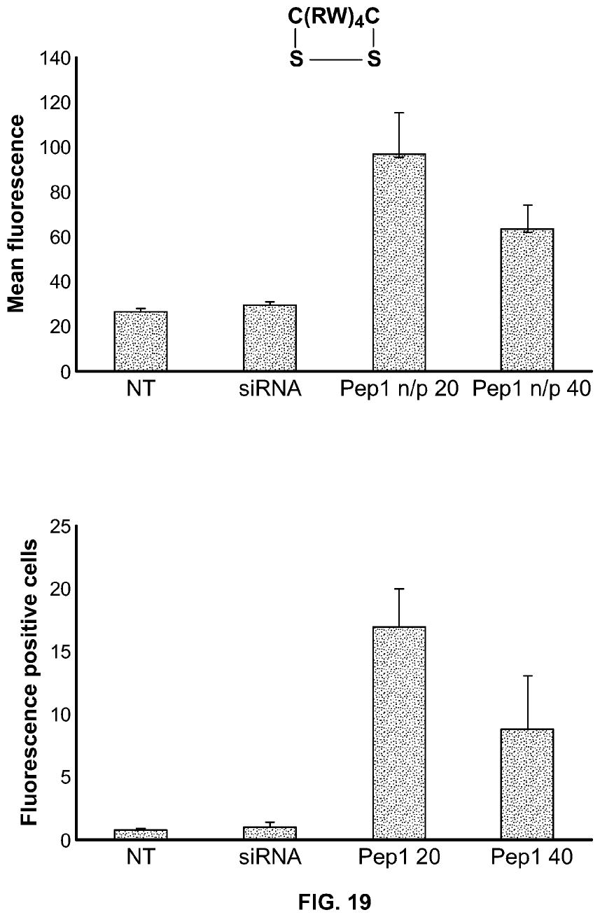

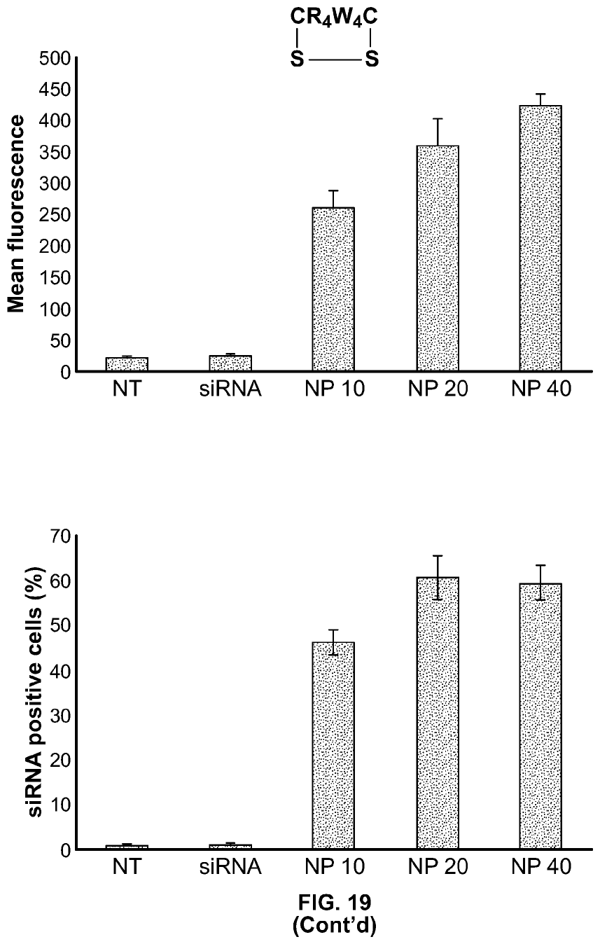

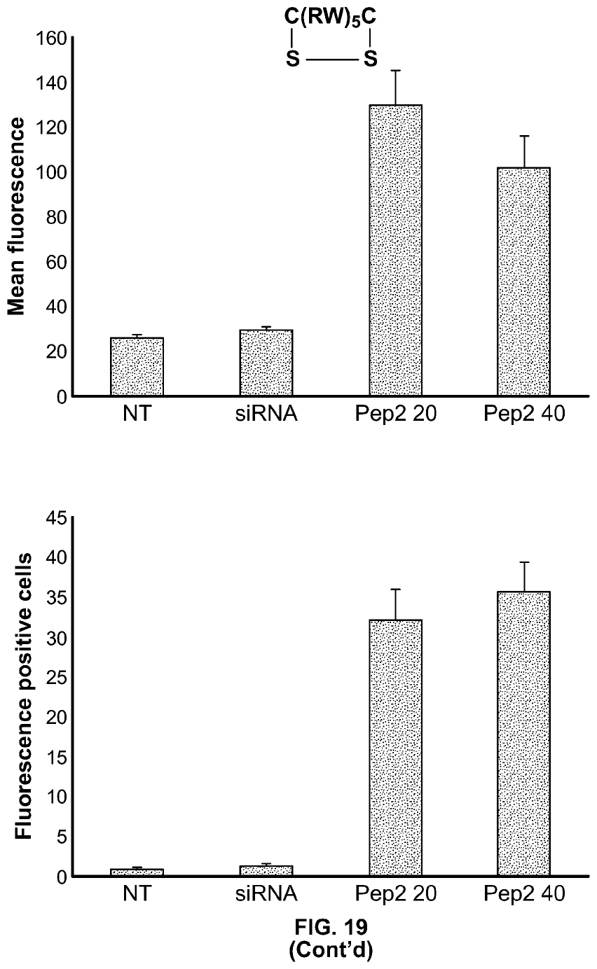

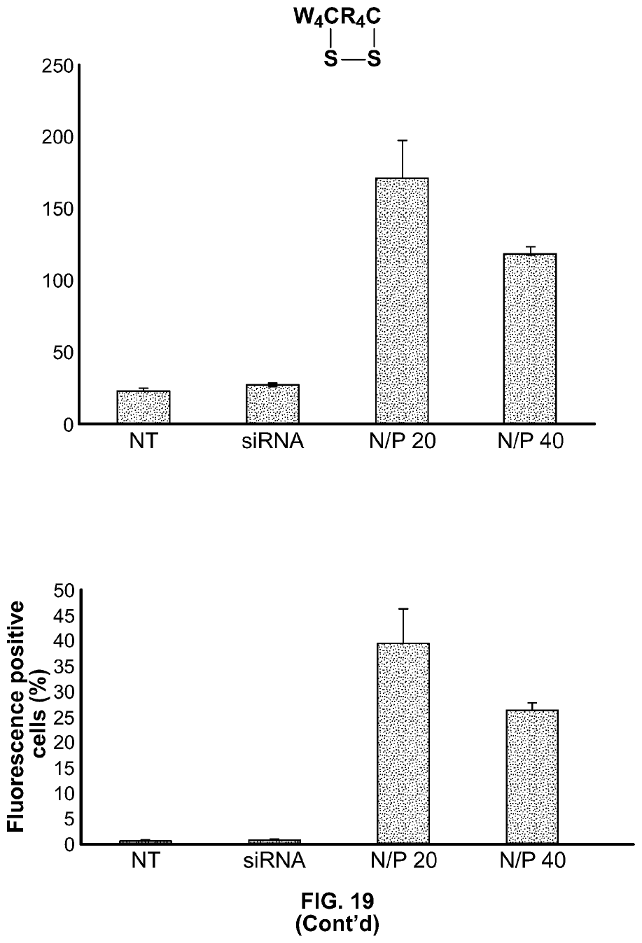

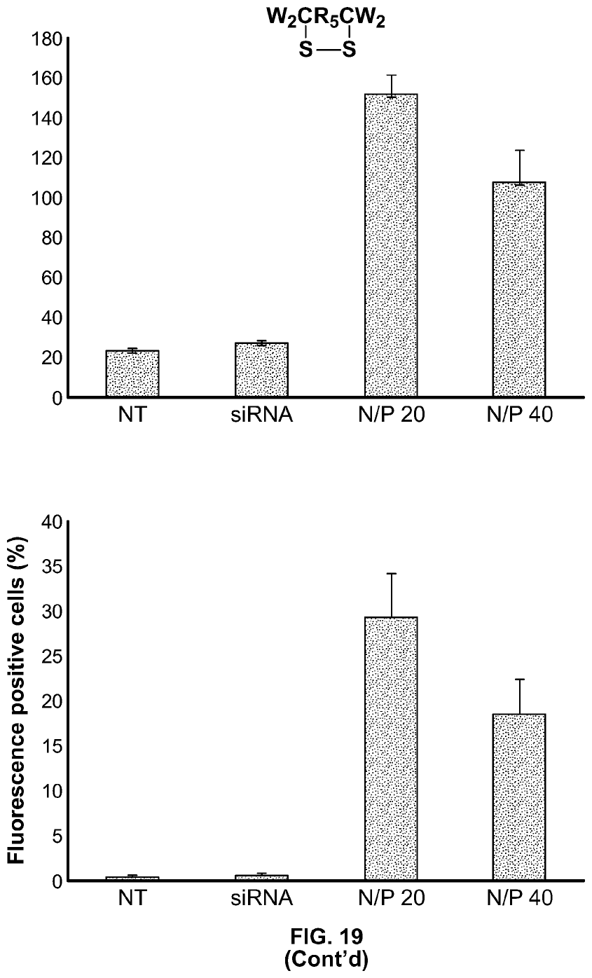

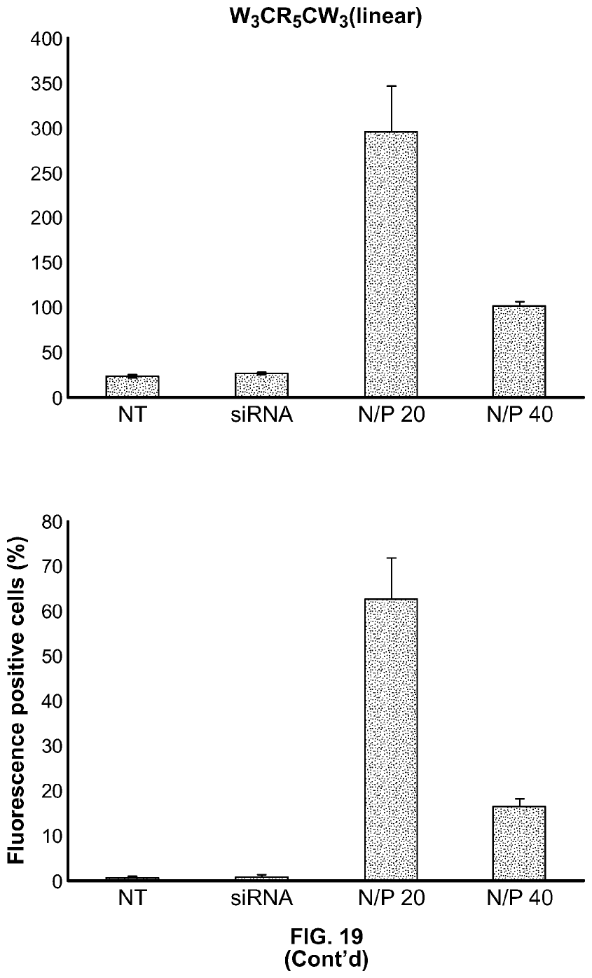

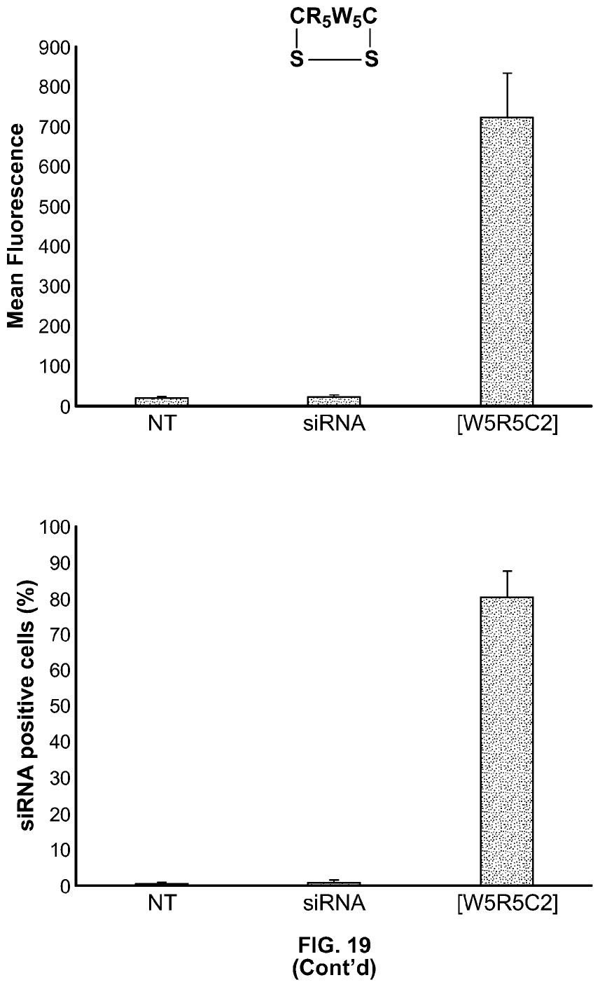

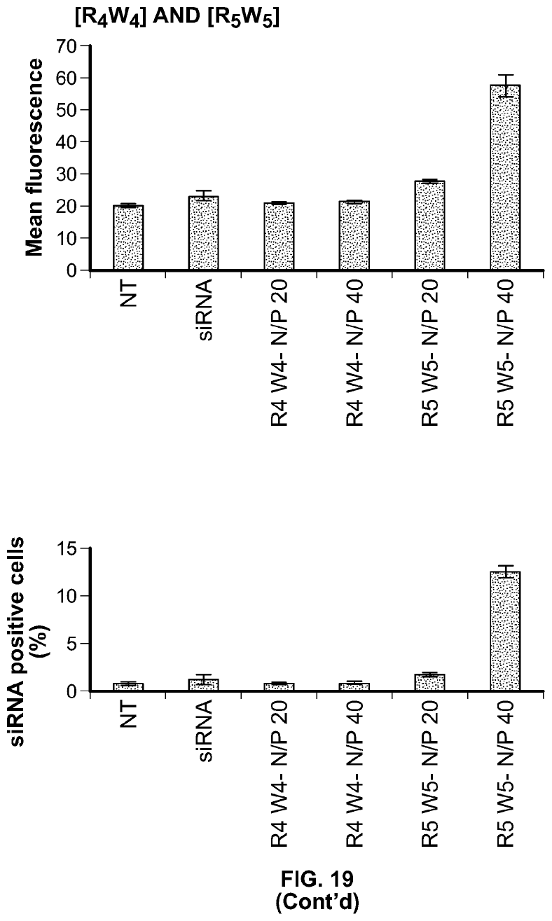

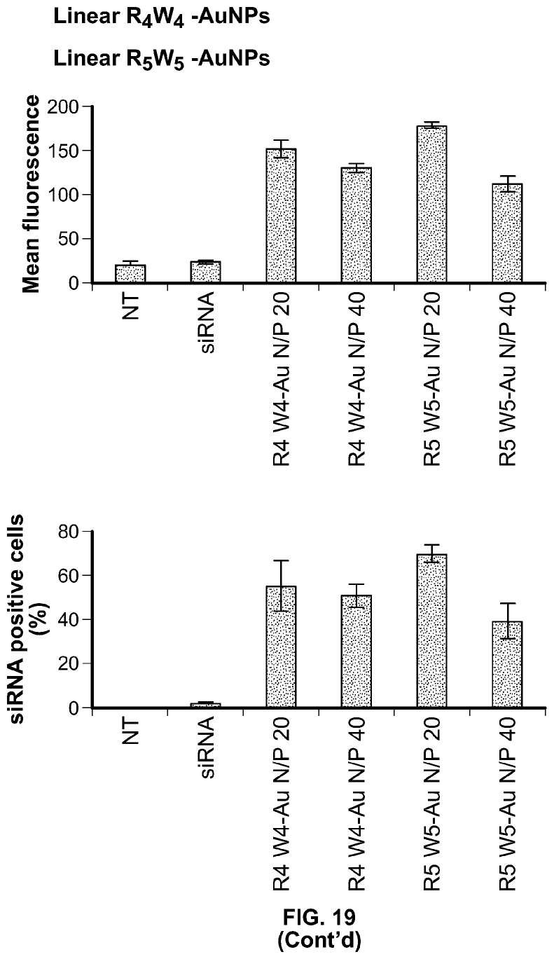

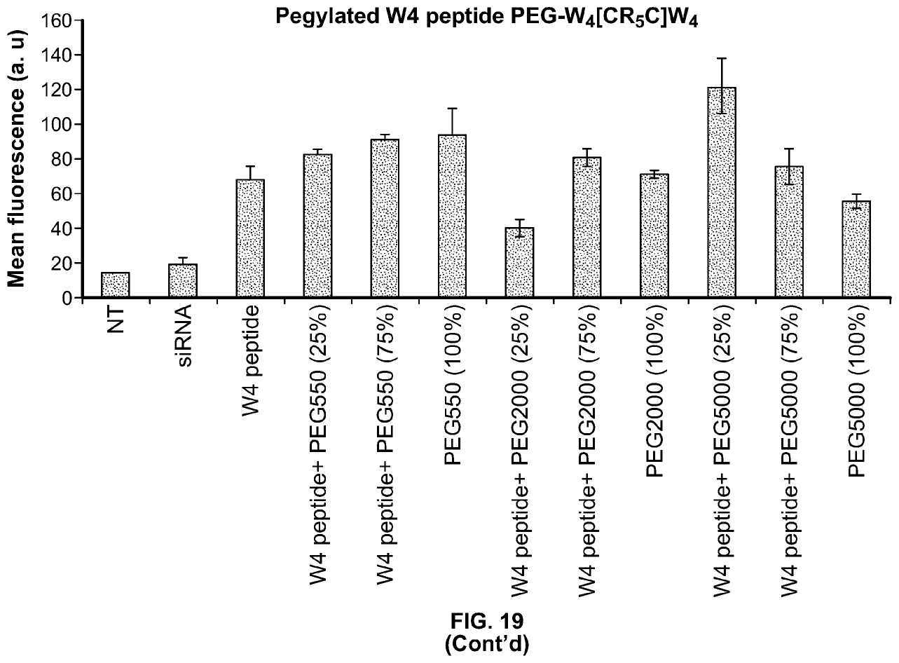

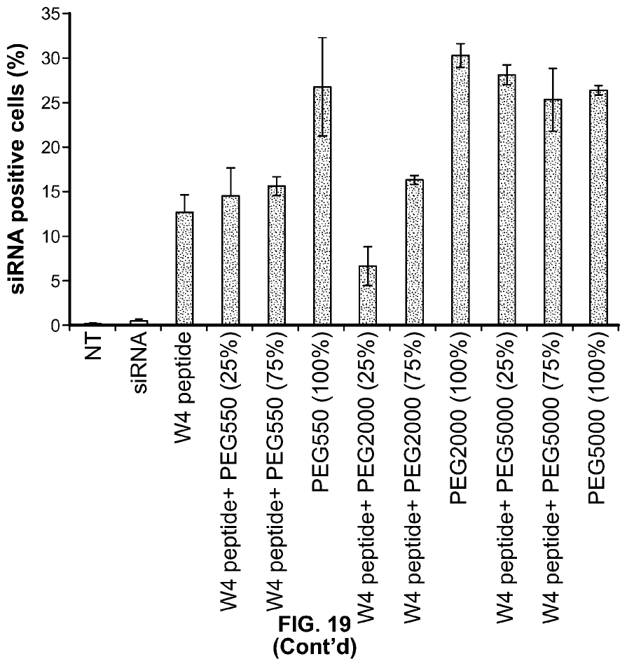

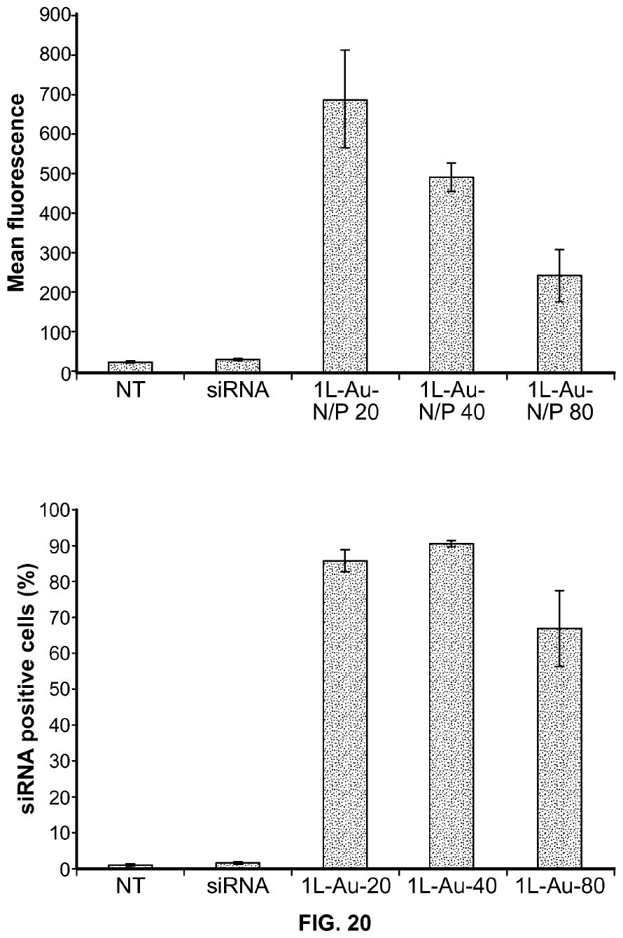

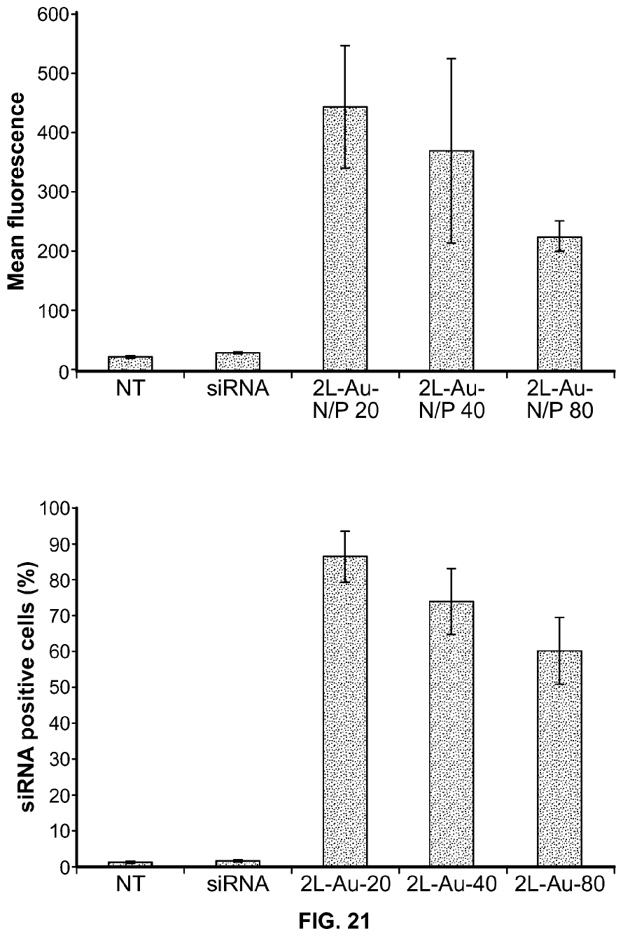

The ability of peptides to deliver siRNA into the MDA-MB-231 cells was evaluated using FAM-labeled scrambled siRNA and analysis by flow cytometry (FIG. 19) according to the previously reported procedure by us (Mozaffari et al., 2019). MDA-MB-231 cells were used for these studies and were seeded in 24-well plates (≈200,000 cells per well). After the addition of the siRNA complexes to cell culture media, cells were incubated at 37° C. and standard growth conditions for 24 hours. After the incubation period, and for flow cytometry studies, cells were washed with clear HBSS (x2), trypsinized, and fixed using 3.7% formaldehyde solution. Suspended cells were analyzed with a BD-FACSVerse (BD Biosciences; San Jose, Calif.) using the FITC channel to quantify cell-associated fluorescence. After each flow cytometry analysis, the percentage of cells with fluorescence signal and the mean fluorescence of the cell population were calculated based on the calibration of the signal gated with non-treated cells (as the negative control), so that the auto-fluorescence would be ≈1% of the population.

The peptides showed significant siRNA uptake, which was significantly higher at a peptide:siRNA ratio of 20:1 and 40:1 after 24 h incubation. The mean fluorescence results and positive fluorescence cells confirmed a similar pattern. The addition of PEGylated peptide (25%) in the native peptide solution enhances the uptake of siRNA.

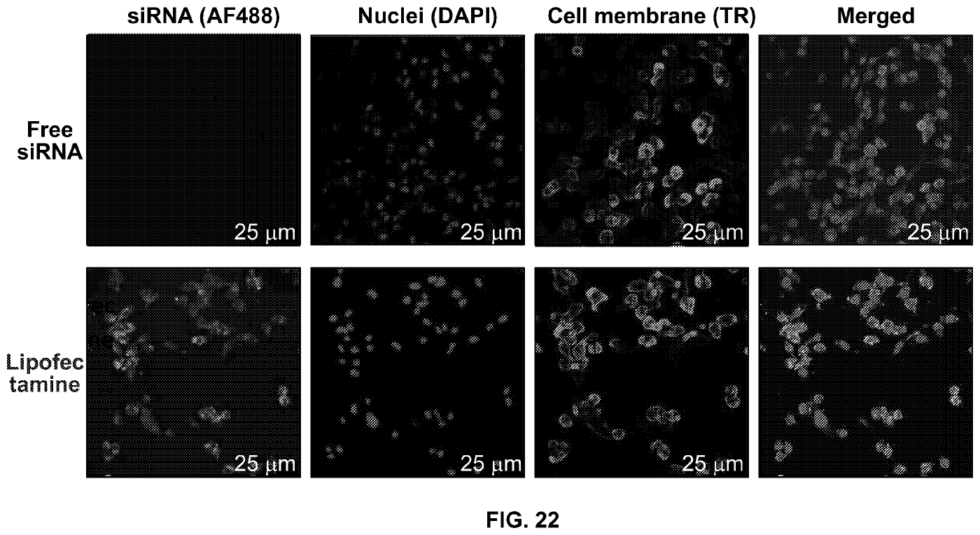

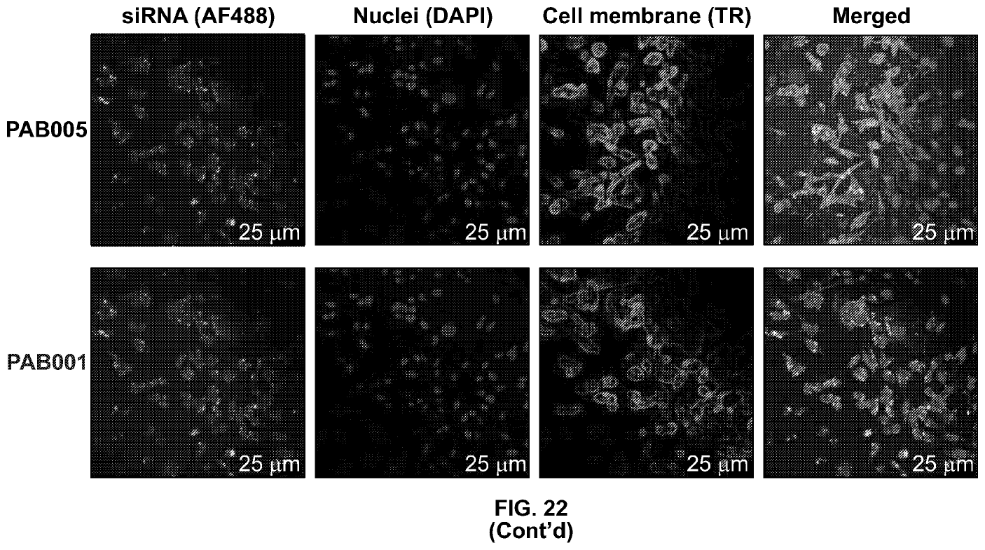

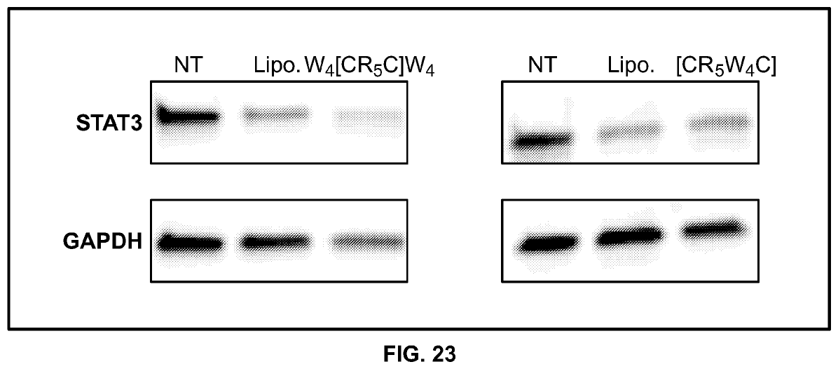

The efficiency of siRNA uptake in this cell line was also confirmed with confocal microscopy images (FIG. 22). The siRNA silencing efficiency were also evaluated in the same cells targeting signal transducer and activator of transcription 3 (STAT3) as a model protein. STAT3 is a well-studied protein with a central role in JAK2/STAT3 pathway and cancer cell proliferation and survival. The expression level of the targeted protein was assessed using Western Blot, which showed comparable silencing efficiency of lead peptides to Lipofectamine™ 2000 (FIG. 23).

The siRNA silencing efficiency were also evaluated in the same cells targeting signal transducer and activator of transcription 3 (STAT3) as a model protein. STAT3 is a well-studied protein with a central role in JAK2/STAT3 pathway and cancer cell proliferation and survival. The expression level of targeted protein was assessed using Western Blot, which showed comparable silencing efficiency of lead peptides to Lipofectamine™ 2000 (FIG. 23).

Methodology for Cell-Based Assays. Cell Lines: The human breast cancer cell line MDA-MB-231 was purchased from American Type Culture Collection (ATCC; Manassas, Va.). MDA-MB-231 was cultured in the DMEM medium. The media was supplemented with 10% (v/v) fetal bovine serum, 100 U/mL penicillin, and 100 μg/ml streptomycin. Cells were maintained in the normal condition of 37° C. and 5% CO₂ under a humidified atmosphere and were sub-cultured when 80-100% confluent.

Toxicity of the Peptide/siRNA Complexes: The safety profile of the peptides was evaluated in two different human cancer cell lines, by exposing the cells to the peptides alone and in peptide/siRNA complexes according to the previously reported procedure (Mozaffari et al., 2019). Confluent cultures (≈800,000 cells/mL) of MDA-MB-231 cancer cell lines were seeded in 96-well plates. After 24 h, cells were exposed to different concentrations of peptide solutions or peptide/siRNA complexes. The final concentration of scrambled siRNA was 36 nM in all cytotoxicity studies performed with peptide/siRNA complexes. The cells were then incubated for 48 hours at 37° C. with controlled CO₂ and humidity. A standard 3-(4,5-dimethylthiazol-2-yl)-2,5-diphenyltetrazolium bromide (MTT) assay was performed after the incubation period to determine the cell viability as a percentage of untreated (NT) cells.

Internalization of siRNA into Human Cancer Cell Lines: The capability of peptides in internalizing siRNA into human cells was evaluated using FAM-labeled scrambled siRNA and flow cytometry according to the previously reported procedure (Mozaffari et al., 2019). MDA-MB-231 cells were used for these studies and were seeded in 24-well plates (≈200,000 cells per well). After the addition of the siRNA complexes to cell culture media, cells were incubated at 37° C. and standard growth conditions for 24 hours. After the incubation period, and for flow cytometry studies, cells were washed with clear HBSS (x2), trypsinized, and fixed using 3.7% formaldehyde solution. Suspended cells were analyzed with a BD-FACSVerse (BD Biosciences; San Jose, Calif.) using the FITC channel to quantify cell-associated fluorescence. After each flow cytometry analysis, the percentage of cells with fluorescence signal and the mean fluorescence of the cell population were calculated based on the calibration of the signal gated with non-treated cells (as the negative control), so that the auto-fluorescence would be ≈1% of the population.

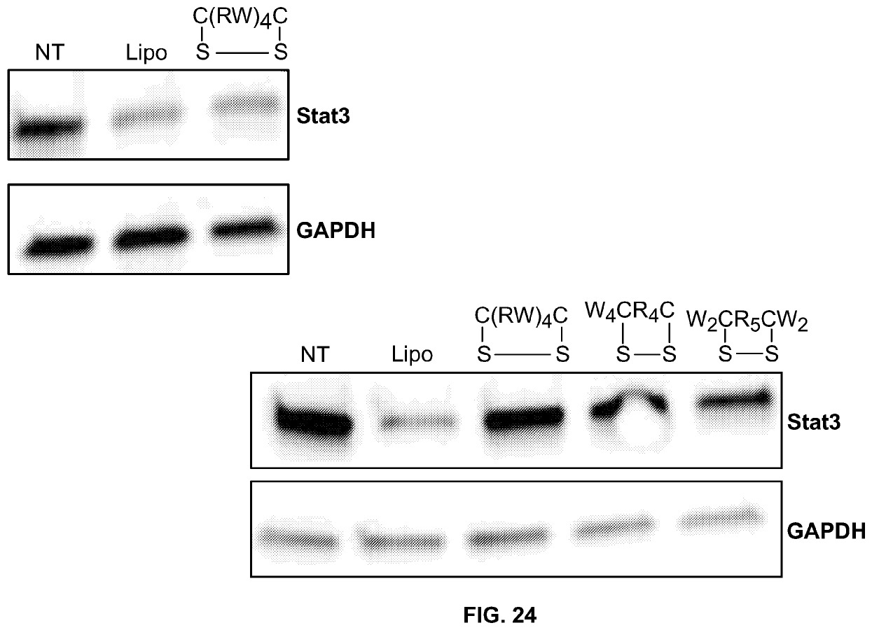

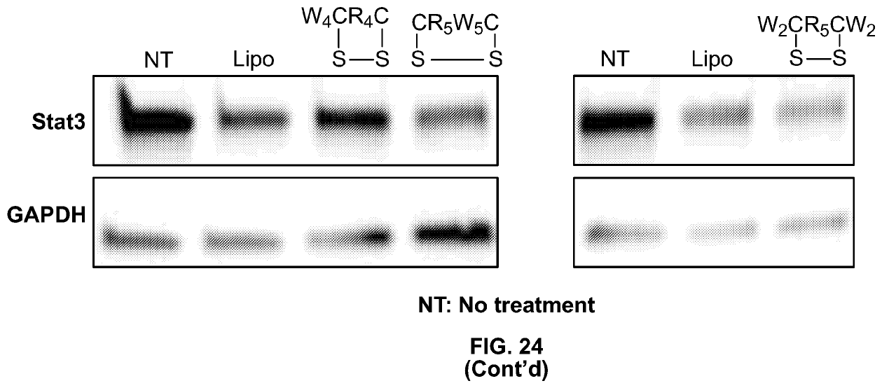

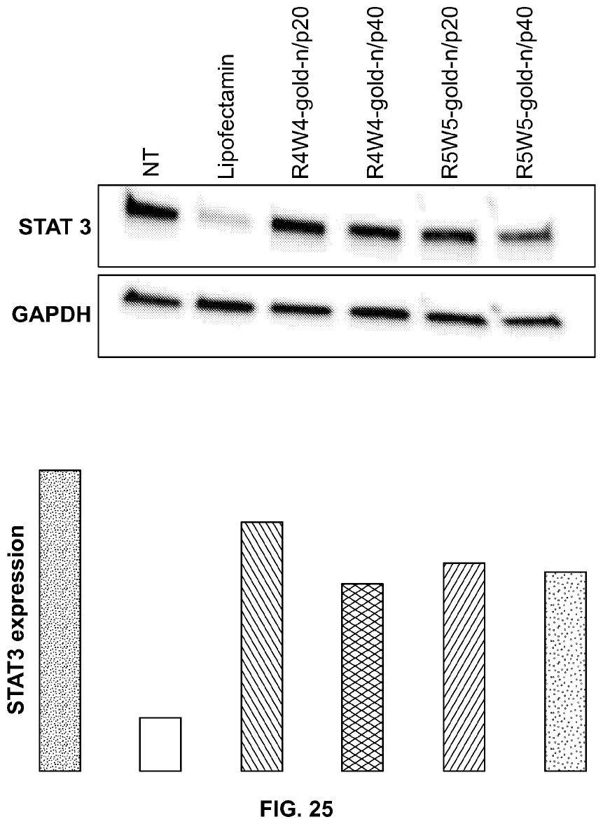

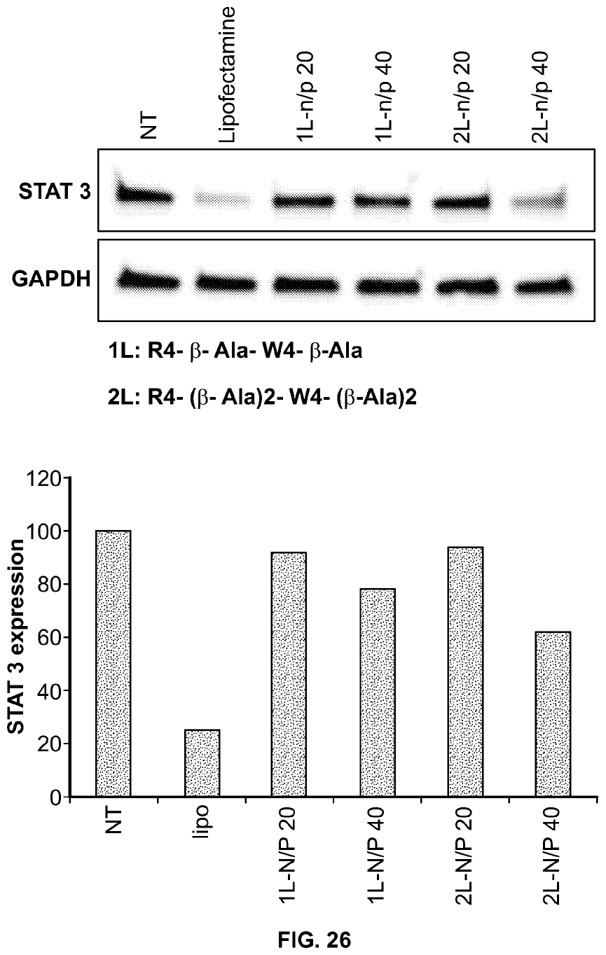

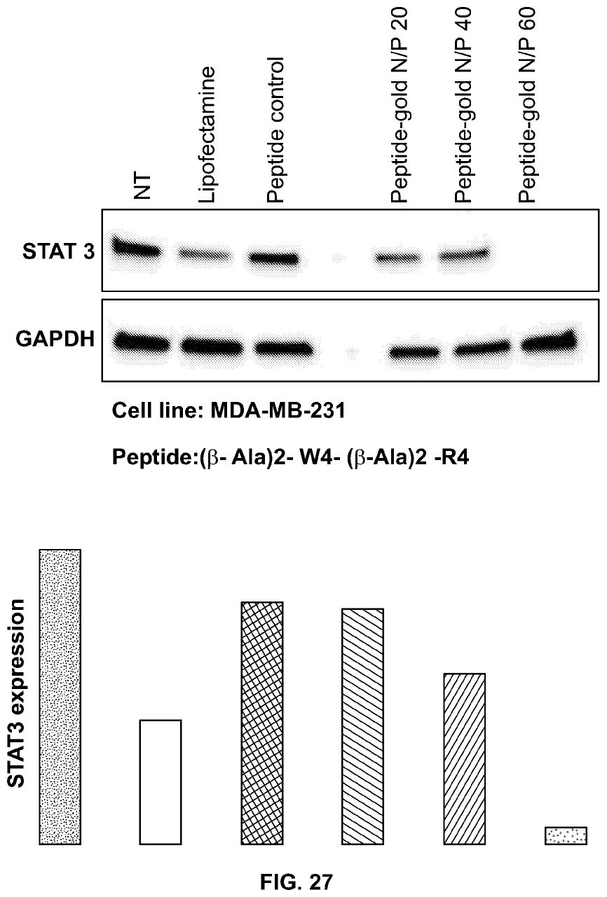

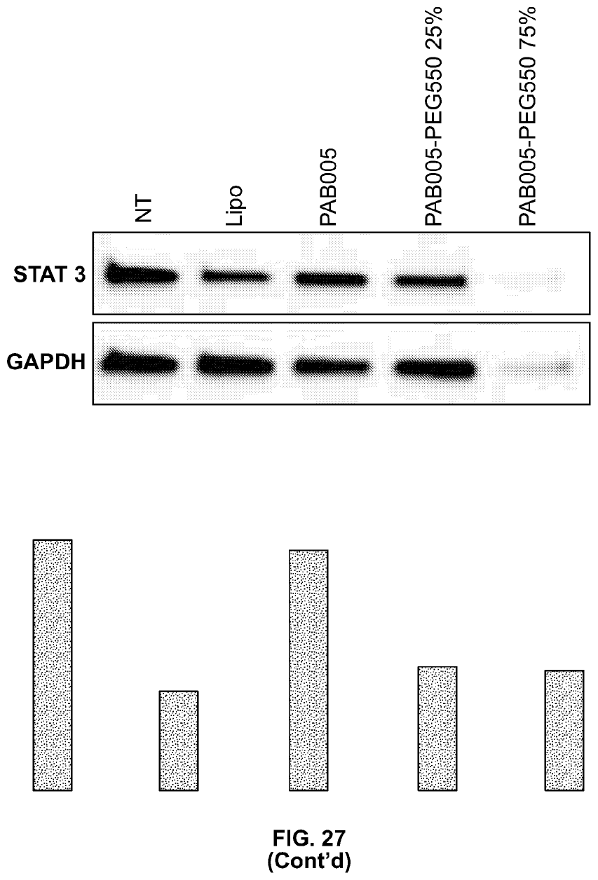

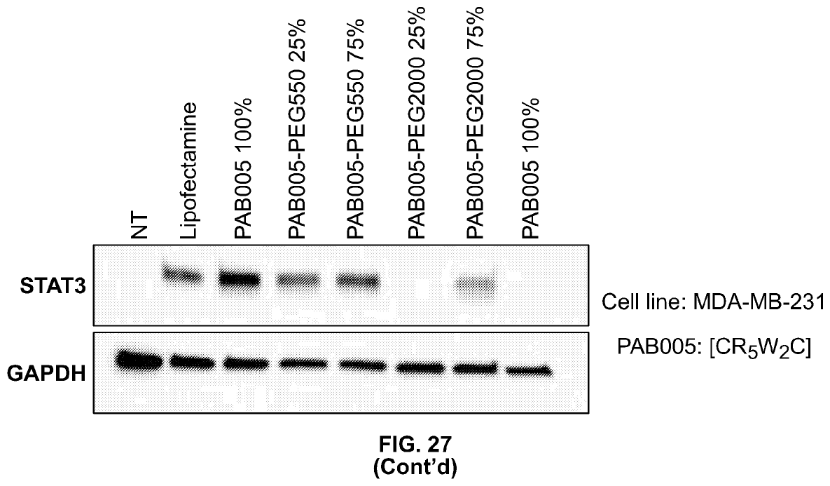

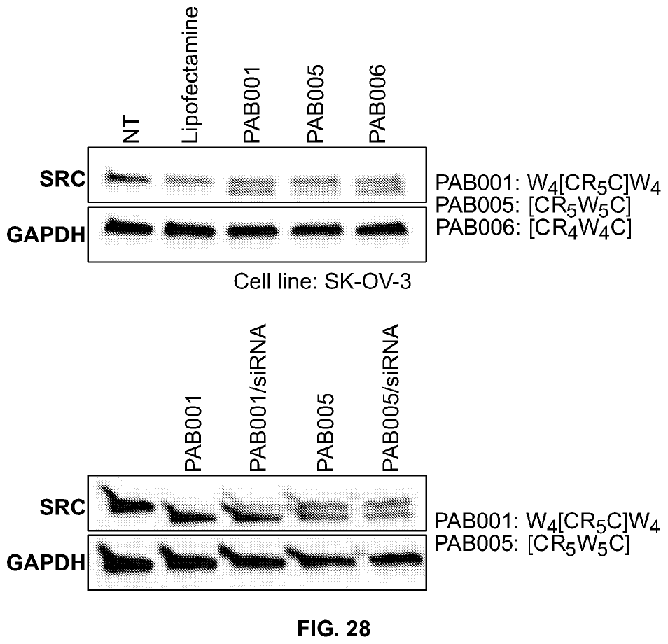

Gene Silencing. Signal Transducer and Activator of Transcription 3 (STAT3) protein was selected as a model protein to demonstrate the in vitro silencing efficiency of the designed modified peptides. STAT3 has been shown to play a major role in the proliferation and survival of different types of cancer, including breast cancer, and may even be involved in resistance against molecularly targeted drugs (Bousoik “Do Inventors Know Jack” About JAK? A Closer Look at JAK/STAT Signaling Pathway. Front Oncol, 2018, 8:287). Along with Janus Kinase 2 (JAK2), STAT3 is among the major proteins involved in this inter-pathway crosstalk, and latest reports have led to the elucidation of a key role of the JAK/STAT signaling pathway in the development, proliferation, differentiation, and survival of cancer cell, and in fact, Vogelstein et at have included JAK/STAT pathway among 12 core cancer pathways (Cancer genome landscapes. Science, 2013, 339:1546-58). The effect of STAT3 activation on Ras and PI3K/Akt pathways and the connections of JAK2 to PI3K and ERK pathways are examples of these inter-pathway cross-talks. Several peptides when used with siRNA, exhibited significant silencing of Stat3 similar to or better than lipofectamine (FIG. 24).

Protein Quantification (Western Blot). The expression of the targeted protein was analyzed by western blot. Cell lysates were prepared according to the standard protocol using RIPA buffer. Briefly, cells exposed to siRNA complexes were collected by trypsinization after 48 h of exposure and were centrifuged at 600-800 RPM for 5 min. The supernatant was discarded, and the cell pellet was washed three times with ice-cold PBS. The pellet was resuspended in RIPA buffer (100 μL of buffer for 25 μL of cell pellet), and the cell lysates were then incubated on ice for one hour, during which the tubes were sonicated for 3 min every 10 min. The tubes were then centrifuged for 15 min at 12,000×g (at 4° C.). Microtubes were pre-cooled to transfer the supernatant, and total protein concentration was determined using BSA assay. Briefly, 200 μL of work reagent (50:1 A:B) was added to 25 μL of standard and unknown samples in triplicate into a 96-well plate, and the plate was mixed on a plate shaker for 30 s. The plate was then incubated at 37° C. and 5% CO₂ for 30 min. The absorbance was measured at 562 nm using SpectraMAX M5 microplate reader. Protein (25 μg) was loaded per well in a 10% Mini-PROTEAN™ TGX Stain-Free™ Protein gel using electrophoresis buffer (0.192. M glycine, 25 mM Tris, 0.1% SDS), and the electrophoresis was run for 60 min with 100 V. The gel was then transferred onto a Trans-Blot™ Turbo™ Mini PVDF membrane (Catalog no. 1704156). Membranes were blocked in BSA 5% for 3 h, and then incubated overnight (at 4° C.) with the primary anti-body (1:1000 in TBS-T). The membrane was then washed with TBS-T three times (5 min each time) and was subsequently incubated with the secondary HRP-linked antibody (1:1000 in TBS-T) for 1 h, followed by the washing steps. Detection was done by ECL Detect Kit using CherniDoc imager (Bio-Rad).

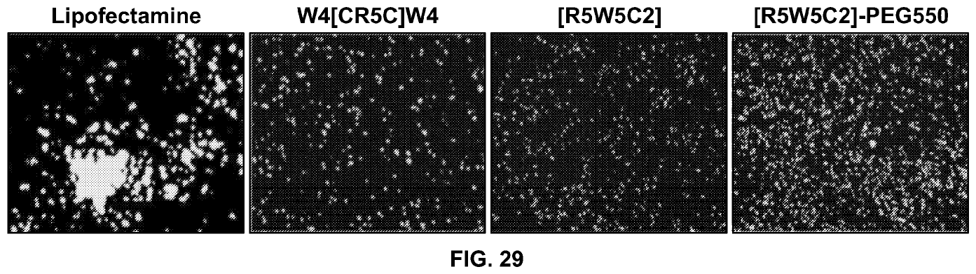

The peptide/plasmid complexes formed with different peptides were able to effectively transfect HEK293 cells with Green Fluorescence Protein (GFP)-expressing plasmid with no apparent toxicity (compared to commercially available Lipofectamine, which causes cell death and aggregation in the culture media (FIG. 29).

It should be apparent to those skilled in the art that many more modifications besides those already described are possible without departing from the concepts herein. The disclosed subject matter, therefore, is not to be restricted except in the spirit of the appended claims. Moreover, in interpreting both the specification and the claims, all terms should be interpreted in the broadest possible manner consistent with the context. In particular, the terms “comprises” and “including” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . , and N, the text should be interpreted as requiring only one element from the group, not A plus N, or B plus N, etc.

What is claimed is:

1. A composition for transferring a nucleic acid into a. living cell, comprising a cyclic peptide comprising a plurality of a positively charged amino acids X and a plurality of a hydrophobic amino acids Y, wherein positively charged amino acids X and hydrophobic amino acids Y are coupled to one another in an alternating fashion between two cysteine residues with a disulfide bridge or N- to C-terminal amide bond.

2. A composition for transferring a nucleic acid into a living cell, comprising a cyclic peptide comprising a plurality of a positively charged amino acids X coupled to one another in a first segment and a plurality of a hydrophobic amino acids Y coupled to one another in a second segment, wherein the first segment and the second segment are coupled to one another between two cysteine residues with a disulfide bridge or N- to C-terminal amide bond.

3. A composition for transferring nucleic acid into a living cell, comprising a peptide selected from the group consisting of a linear peptide (Y)ₙCXₙC(Y)ₙ or (X)ₙCYₙC(X)ₙ, a hybrid cyclic-linear peptide (Y)ₙ[CXₙC](Y)ₙ, a hybrid cyclic-linear peptide (X)ₙ[CYₙC](X)ₙ, a hybrid cyclic-linear peptide (Y)ₙ[KXₙK](Y)ₙ, a hybrid cyclic-linear peptide (X)ₙ[KYₙK](X)ₙ, a linear peptide (X)ₙ(KYₙK)(X)ₙ, and a linear peptide (X)ₙ(Yₙ)(X), wherein Y is a hydrophobic amino acid, X is a positively-charged amino acid, each n is independently from 1 to 10.

4. A composition for transferring nucleic acid into a living cell, comprising a linear or cyclic peptide comprising a plurality of positively charged amino acids X and a plurality of hydrophobic amino acids Y separated from one another by one or more β-alanines.

5. The composition of any one of claims 1-4, wherein the peptide is a linear peptide.

6. The composition of any one of claims 1-4, wherein the peptide is a cyclic peptide having a cyclic structure, wherein the cyclic structure includes a disulfide bridge, a peptide bond generated between N-terminal and C-terminal amino acids of a linear peptide precursor, or both.

7. The composition of any one of claims 1-4, wherein the peptide is a hybrid peptide comprising a cyclic portion having a cyclic structure and a linear portion.

8. The composition of any one of claims 1-7, wherein the peptide further comprises a plurality of cysteine residues.

9. The composition of claim 8. wherein the peptide is the cyclic peptide, comprising a disulfide bond between two of the plurality of cysteine residues, wherein the disulfide bond forms part of the cyclic structure.

10. The composition of claim 8, wherein the peptide is the hybrid peptide, comprising a disulfide bond between two of the plurality of cysteine residues, wherein the disulfide bond forms part of the cyclic structure.

11. The composition of any one of claims 1-10, wherein the charged amino acid X is selected from the group consisting of 1-arginine, 1-lysine, 1-histidine, d-histidine, d-arginine, d-lysine, ornithine, a modified histidine residues with a hydrocarbon side chain, C3-Arginine, C4-Arginine, diaminopropionic acid (Dap), diaminobutyric acid (Dab), an amino acid comprising a free side-chain amino or guanidine group, a modified arginine, and a modified lysine.

12. The composition of any one of claims 1-11, wherein the hydrophobic amino acid Y is selected from the group consisting of 1-tryptophan, d-tryptophan, 1-phenylalanine, d-phenylalanine, 1-isoleucine, d-isoleucine, p-phenyl-1-phenylalanine (Bip), 3,3-diphenyl-1-alanine (Dip), 3(2-naphthyl)-1-alanine (NaI), 6-amino-2-naphthoic acid, 3-amino-2-naphthoic acid, 1,2,3,4-tetrahydronorharmane-3-carboxylic acid, 1,2,3,4-tetrahydro-3-isoquinolinecarboxylic acid (Tic-OH), 1,2,3,4-tetrahydro-3-isoquinolinecarboxylic acid, a modified d- or 1-tryptophan residue, an N-alkyl tryptophan, an N-aryl tryptophan, a substituted d- or 1-tryptophan residue, 5-hydroxy-L-tryptophan, 5-methoxy-L-tryptophan, 6-chloro-L-tryptophan, a fatty amino acid having a formula NH₂-(CH₂)ₓ-COOH wherein x=1-20, and an N-heteroaromatic amino acid.

13. The composition of any one of claims 1-12, wherein the peptide comprises a non-peptide bond linking adjacent amino acids.

14. The composition of claim 13, wherein the non-peptide bond is selected from the group consisting of a thioamide bond, an N-methyl bond, and a CH₂-NH bond.

15. The composition of any one of claims 1-14. wherein the peptide comprises a modification to a side chain of a charged amino acid X or a hydrophobic amino acid Y.

16. The composition of claim 15, wherein the modification is PEGylation.

17. A composition for the delivery of a nucleic acid into a living cell, comprising:
a peptide of any one of claims 1-16; and
a nanoparticle.

18. The composition of claim 17, wherein the nanoparticle comprises gold or silver.

19. The composition of claim 17 or 18, wherein the nanoparticles is a peptide-capped nanoparticle.

20. The composition of any one of claims 17-19, further comprising the nucleic acid.

21. The composition of claim 20, wherein the nucleic acid is an RNA.

22. The composition of claim 21, wherein the RNA is an siRNA.

23. The composition of claim 20, wherein the nucleic acid is an DNA.

24. The composition of claim 20, wherein the nucleic acid is a part of CRISPR.

25. The composition of any one of claims 17-20, wherein the composition is formulated for topical, oral, injection, or nasal administration.

26. A composition for the delivery of nucleic acid into a living cell, comprising:
a peptide of any one of claims 1-16; and the nucleic acid.

27. The composition of claim 20, wherein the composition is formulated for topical, oral, injection, or nasal administration.

28. A method of delivering a nucleic acid into a living cell, comprising:
contacting a peptide of any one of claims 1-16 with the nucleic acid to form a peptide:nucleic acid complex; and
contacting the cell with the peptide:nucleic acid complex.

29. The method of claim 28, further comprising a step of isolating the peptide:nucleic acid complex prior to contacting the cell.

30. The method of claim 28 or 29, wherein the peptide:nucleic acid complex further comprises a nanoparticle.

31. The method of claim 30, wherein the nanoparticle comprises gold or silver.

32. The method of any one of claims 28-31, wherein the cell is selected from the group consisting of a bacterial cell, a plant cell, an animal cell, and a fungal cell.

33. A method of treating a disease state in an individual, comprising:
contacting a peptide of any one of claims 1-16 with a nucleic acid to form a peptide:nucleic acid complex; and
contacting a cell obtained from the individual with the peptide:nucleic acid complex.

34. The method of claim 33, further comprising a step of isolating the peptide: nucleic acid complex prior to contacting the cell.

35. The method of claim 33 or 34, wherein the peptide:nucleic acid complex further comprises a nanoparticle.

36. The method of claim 35, wherein the nanoparticle comprises gold or silver.

37. The method of any one of claims 33-36, wherein the disease state is selected from the group consisting of cancer, a central nervous system disorder, an autoimmune disease, a genetic disease, a proliferative disease, a hematological disease, an inflammatory disease, a gastrointestinal disease, a liver disease, a lung disease, a kidney disease, a spleen disease, a familial amyloid neuropathy, pain, a metabolic disorder, a psychiatric disorder, a bacterial infection, a viral infection, and a fungal infection.

38. The method of one of claims 33-37, wherein the peptide:nucleic acid complex is formulated as a pharmaceutical, wherein the pharmaceutical is suitable for at least one of topical, oral, injection, nasal, vaginal, and rectal administration.

39. The method of one of claims 33-38 wherein treating comprises at least one of prophylaxis and treatment of active disease.

40. A cosmetic preparation comprising:
a peptide of any one of claims 1-16; and
a cosmetically acceptable vehicle.

41. The cosmetic preparation of claim 40, further comprising a nucleic acid.

42. The cosmetic preparation of claim 41, wherein the nucleic acid is an RNA.

43. The cosmetic preparation of claim 42, wherein the RNA is an siRNA.

44. The cosmetic preparation of claim 41, further comprising a DNA.

45. The cosmetic preparation of any one of claims 40-44 further comprising a nanoparticle.

46. The cosmetic preparation of claim 45, wherein the nanoparticle comprises gold or silver.

47. The cosmetic preparation of any one of claims 40-46 wherein the cosmetic preparation is formulated as a micellar suspension, a lotion, a cream, a masque. an ointment, a gel, or a shampoo.

48. Use of a composition of any one of claims 1-16 in a food industry.

49. The use of claim 48, wherein the food industry is agriculture,

50. The use of claim 49, wherein the use is as a pesticide or herbicide.

51. The use of claim 48-50, wherein the composition further comprises a nanoparticle.

52. The use of claim 51, wherein the nanoparticle comprises gold or silver.

53. A composition for transferring nucleic acid into a living cell, comprising a linear peptide comprising a plurality of hydrophobic residues coupled to two chains of hydrophilic residues through two cysteine residues.

54. A composition for transferring nucleic acid into a living cell, comprising a linear peptides comprising a plurality of hydrophilic residues coupled to two chains of hydrophobic residues through two cysteine residues.

55. A composition for transferring nucleic acid into a living cell, comprising a cyclic peptide comprising a plurality of hydrophobic residues coupled to two chains of hydrophilic residues through two cysteine residues.

56. A composition for transferring nucleic acid into a living cell, comprising a cyclic peptide comprising a plurality hydrophilic residues coupled to two chains of hydrophobic residues through two cysteine residues.

57. A composition for transferring nucleic acid into a living cell, comprising a linear peptide comprising hydrophobic residues coupled to two chains of hydrophilic residues through two lysine residues.

58. A composition for transferring nucleic acid into a living cell, comprising a linear peptide containing hydrophilic residues coupled to two chains of hydrophobic residues through two lysine residues.

59. A composition for transferring nucleic acid into a living cell, comprising a cyclic peptide comprising a plurality of hydrophobic residues attached to two chains of hydrophilic residues through two lysine residues.

60. A composition for transferring nucleic acid into a living cell, comprising a cyclic peptide comprising a plurality of hydrophilic residues coupled to two chains of hydrophobic residues through two lysine residues.

61. The composition of any one of claims 1-4 for the delivery of a nucleic acid into a living cell.

62. The composition of any one of claims 1-4 for transferring one or more siRNA into a living cell.

63. The composition of any one of claims 1-4 for transferring one or more DNA into a living cell.

64. The composition of any one of claims 1-4 for transferring one or more plasmid into a living cell.

65. The composition of any one of claims 1-4 for transferring one or more CRISPR into a living cell.

66. The composition of any one of claims 1-4 for transferring one or more hairpin RNA into a living cell.

67. The composition of any one of claims 1-4 for transferring one or more micro RNA into a living cell.

68. The composition of any one of claims 1-4 for transferring one or more oligonucleotides into a living cell.

69. The composition of any one of claims 1-4 for transferring any negatively charged molecules, such as phosphopeptides and phosphoproteins.

70. The composition of any one of claims 1-4 that is used as transfecting agents.

71. The method of any one of claims 33-36 for treatment or mitigating viral infections.

72. The method of any one of claims 33-36, wherein the peptide-nucleic acid complex is used as a vaccine for prevention of disease.

73. The method of claim 33, wherein treating comprises at least one of prophylaxis and treatment of active disease.

73. The composition of any one of claims 1-4, wherein the peptide is pegylated.

74. The composition of any one of claims 1-4, wherein the peptide is attached to a targeting moiety directly or through conjugation with PEG.
